(19) United States
(12) Patent Application Publication
(10) Pub. No.: US 2026/0062742 A1
(43) Pub. Date: Mar. 5, 2026

(54) RECOMBINANT LIGASES AND USES THEREOF

(71) Applicant: Singular Genomics Systems, Inc., San Diego, CA (US)

(72) Inventors: Souad Naji, La Jolla, CA (US); Joshua Catungal Corpuz, San Diego, CA (US); Jamie PP Do, San Diego, CA (US); Eli N. Glezer, Del Mar, CA (US); Henry Hoi-Chung Kwan, San Diego, CA (US); Tung Thanh Le, San Diego, CA (US)

(21) Appl. No.: 19/199,539

(22) Filed: May 6, 2025

Related U.S. Application Data
(60) Provisional application No. 63/644,355, filed on May 8, 2024.
(60) Provisional application No. 63/651,109, filed on May 23, 2024.
(60) Provisional application No. 63/664,987, filed on Jun. 27, 2024.
(60) Provisional application No. 63/705,332, filed on Oct. 9, 2024.
(60) Provisional application No. 63/771,580, filed on Mar. 13, 2025.

(51) Int. Cl. C12Q 1/6869; C12N 9/00; C12N 9/96

(52) U.S. Cl. CPC C12Q 1/6869; C12N 9/93; C12N 9/96; C12Y 605/01001

(57) ABSTRACT

Disclosed herein, inter alia, are ligases, kits, and methods of use thereof.

### CROSS-REFERENCES TO RELATED APPLICATIONS

This application claims the benefit of U.S. Provisional Application No. 63/644,355, filed May 8, 2024, U.S. Provisional Application No. 63/651,109, filed May 23, 2024, U.S. Provisional Application No. 63/664,987, filed Jun. 27, 2024, U.S. Provisional Application No. 63/705,332, file Oct. 9, 2024, and U.S. Provisional Application No. 63/771,580, filed Mar. 13, 2025, each of which are incorporated herein by reference in their entirety and for all purposes.

### SEQUENCE LISTING

The instant application contains a Sequence Listing which has been submitted electronically in XML format and is hereby incorporated by reference in its entirety. Said XML copy, created on Apr. 23, 2025, is named 00620001US.xml, and is 28,418 bytes in size.

### BACKGROUND

Ligases catalyze the joining of the 5′-phosphorus-terminated strand with the 3′-hydroxyl-terminated strand, specifically within double-stranded polynucleotides, ensuring that the enzyme operates on double-stranded DNA or RNA molecules. A commercially available ligase, PCBV-1 DNA ligase isolated from the Chlorella virus, is widely used due to the innate ability of the PCBV-1 DNA ligase to ligate single-stranded DNA polynucleotides splinted by an RNA molecule. In addition, PCBV-1 DNA ligase has been shown to exhibit high substrate specificity for nicked substrates. Despite the importance of PCBV-1 DNA ligases in various workflows, the utility of PCBV-1 DNA ligase is limited due to its low thermostability and propensity to aggregate in solution. Efforts to develop a robust, thermostable ligase capable of ligating single-stranded DNA polynucleotides that are hybridized onto a complementary RNA molecule remains a challenge. Disclosed herein, inter alia, are solutions to these and other problems in the art.

### BRIEF SUMMARY

In an aspect is provided a ligase and methods of use thereof. In embodiments, the ligase includes one or more amino acid substitutions as described herein. In embodiments, the ligase includes an amino acid sequence that is at least 80%, 85%, 90%, 95%, or 99% identical to SEQ ID NO:1. In embodiments, the ligase includes one or more hydrophilic polymers (e.g., PEG).

In an aspect is provided a ligase including an amino acid sequence that is at least 80%, 85%, 90%, 95%, or 99% identical to SEQ ID NO:1 attached (e.g., covalently attached) to a polynucleotide-binding polypeptide. In embodiments, the polynucleotide-binding polypeptide includes a sequence that is at least 80%, 85%, 90%, 95%, or 99% identical to a continuous 20 nucleotide sequence within SEQ ID NO:3.

In an aspect is provided a kit including a ligase described herein. In embodiments, the kit includes a wild-type ligase or variant thereof. In embodiments, the kit includes a ligase useful for circularizing template polynucleotides. For example, such a kit further includes the following components: (a) reaction buffer for controlling pH and providing an optimized salt composition for the ligase described herein and (b) ligation enzyme cofactors. In embodiments, the kit further includes instructions for use thereof.

In an aspect is provided a method for detecting polynucleotide sequences including: (a) hybridizing a first polynucleotide sequence to a nucleic acid molecule and hybridizing a second polynucleotide sequence to the nucleic acid molecule; (b) ligating the first polynucleotide sequence and the second polynucleotide sequence together with a ligase to generate a ligated product, wherein the ligase includes an amino acid sequence that is at least 85% identical to SEQ ID NO:1; and (c) detecting the ligated product. In embodiments, the first polynucleotide sequence and the second polynucleotide sequence are part of the same single-stranded polynucleotide sequence, which may be ligated to form a circular polynucleotide. In embodiments, the ligated product includes a barcode sequence.

### BRIEF DESCRIPTION OF THE DRAWINGS

FIG. 1 shows an aligned amino acid sequence comparison of a PBCV NYs1 ligase PB-1, bottom, (SEQ ID NO:1) and PBCV-1 Ligase, top, (SEQ ID NO:16).

FIG. 2 illustrates the components of the ligation assay adapted from Tang et al. (Nucleic Acids Res. 2005; 33(11): e97), which was used to measure the ligation ability of the ligases described herein. The molecular beacon, left, includes a loop region, where a portion of the loop is capable of specifically hybridizing with the complementary sequence of Oligo A and the remainder of the loop is capable of specifically hybridizing with the complementary sequence of Oligo B; a fluorophore at the 5′ end of the molecular beacon (depicted as a circle); and a quencher moiety at the 3′ end of the molecular beacon (depicted as a square). A ligatable nick forms as a result of the hybridization of Oligo A and Oligo B to the molecular beacon. In the presence of a ligase (e.g., a ligase variant described herein), the nick between Oligo A and Oligo B is sealed, which results in the opening the molecular beacon, release of the fluorophore from the quencher moiety, and fluorescence of the fluorophore (depicted by the haze behind the fluorophore). Measurement of fluorescence is indicative of the ligation of the nick site by the ligase described herein as fluorescence is quenched when the fluorophore is in close proximity to the quencher moiety prior to the ligation of the nick site.

FIGS. 3A-3B. FIG. 3A provides the gel with bands corresponding to supernatants from PBCV-1 DNA ligase (lanes 3-6) and PB-1 (lanes 7-10) following the evaluation of the thermostability of each ligase at room temperature, 30° C., 37° C., and 45° C. FIG. 3B provides the quantification of the band intensity of the bands shown in lanes 3-10 of FIG. 3A.

FIGS. 4A-4D. FIG. 4A provides a schematic of an mRNA transcript targeted by the DNA padlock probe (PLP), leaving a gap of about 70 nt between the binding sequences of the padlock probe. At low temperatures, there is a higher likelihood of the formation of secondary structures in mRNA transcripts (depicted as a bump near the 3′ foot of the padlock probe) with GC-rich regions (depicted by the rectangle shapes within the gap region of the mRNA transcript). The presence of the secondary structure in mRNA causes the reverse transcriptase (depicted as a cloud) to progress slowly through this region and impedes the gap filling activity of the reverse transcriptase. FIG. 4B provides a schematic of a DNA padlock probe (90 nucleotides in length) and a mRNA transcript targeted by the DNA padlock probe. The gap between the 5′ foot and the 3′ foot is 70 nucleotides in length, which may include GC-rich regions (depicted by the rectangle shapes). FIG. 4C provides bands corresponding to products formed during the gap fill and ligation reactions performed at 37° C. and 30° C. Controls loaded into lane 1 of the gel included single stranded DNA templates of 90 and 200 nucleotides. Controls loaded into lane 2 of the gel included the DNA PLP of 90 nucleotides, the RNA template of 118 nucleotides, and a linear polynucleotide marker of 200 nucleotides; this control represented an unsuccessful gap fill and ligation reaction. Lanes 3-10 corresponded to products formed during gap fill and ligation at 37° C., where lanes 3 and 4 corresponded to products formed in the presence of M-MLV reverse transcriptase H(−) and 10 μM and 4 μM of PBCV-1 DNA ligase, respectively. Lanes 5 and 6 corresponded to products formed in the presence of M-MLV reverse transcriptase H(−) and 10 μM and 4 μM of PB-1, respectively. Lanes 3-6 show bands in the presence of RNase, while lanes 7-10 correspond to bands in the presence of RNase, exonuclease I, and exonuclease III. The presence of RNase, exonuclease I, and/or exonuclease III degrades RNA, linear ssDNA, and linear dsDNA, respectively. Lanes 11-14 corresponded to products formed during gap fill and ligation at 30° C., where lanes 11 and 12 corresponded to products formed in the presence of M-MLV reverse transcriptase H(−) and 10 μM and 4 μM of PBCV-1 DNA ligase, respectively. Lanes 13 and 14 corresponded to products formed in the presence of M-MLV reverse transcriptase H(−) and 10 μM and 4 μM of PB-1, respectively. The desired circular PLP including the 90 nucleotides of the PLP and 70 nucleotides from the gap sequence complementary to the RNA template has a length of 160 nucleotides; the circular DNA PLP migrates at a slower rate compared to the linear DNA molecule of the same size (see, e.g., Stellwagen et al. J Chromatogr A. 2009 Mar. 6; 1216(10): 1917-1929). Linear products corresponding to an overextension event by 24 nucleotides are also shown and are 184 nucleotides in length (i.e., 90 nt from PLP, 70 nt from RNA template, and 24 nt due to overextension). Linear products corresponding to a complete gap filling but unsuccessful ligation were observed with 160 nucleotides; these linear DNA products migrate faster than the circular PLP of the same size. Lastly, linear DNA products formed as a result of MLV reverse transcriptase H(−) pausing at the first GC stretch in the gap was also observed and had 102 nucleotides (i.e., 90 nt from PLP and 12 nt). FIG. 4D reports the circularization % of the DNA PLP by PB-1 and PBCV-1 DNA ligase, where circularization % was calculated as a percentage of the quotient of the intensity of the circularized band and the sum of band intensities from circularized band and overextension band. Intensities of the bands shown in the gel were calculated using ImageJ, and the circularization % are shown for gap fill and ligation reactions with 4 μM of the ligases from FIG. 4C.

FIGS. 5A-5B. Ligase variants (PB-8, PB-9, PB-2, and PB-4) were tested for thermostability at 37° C. (FIG. 5A) and 42° C. (FIG. 5B). Controls included wild type PBCV-1 DNA ligase and PB-1. Ligase variant PB-8 includes an Sso7d fused at the C-terminus of SEQ ID NO:1, while PB-9 includes an Sso7d covalently attached to the N-terminus of SEQ ID NO:1. Ligase variant PB-2 includes a C22S point mutation in SEQ ID NO:1, and ligase variant PB-4 includes a C298S point mutation in SEQ ID NO:1.

FIGS. 6A-6F provide images of a tissue section from bone marrow attached to a flow cell using methods described supra. Each dot on the image depicts a detected RNA molecule via hybridization of a padlock probe specific to the target RNA molecule, ligation of the padlock probe with a ligase described herein, amplification of the ligated padlock probe via rolling circle amplification and sequencing of the amplified product. FIG. 6A shows the detected transcripts in bone marrow, where ligation of the padlock probe was performed by PB-1 at 30° C. FIG. 6B provides the detection of transcripts in bone marrow, where ligation of the padlock probe was performed by PB-9 at 30° C. FIG. 6C shows the detected transcripts in bone marrow, where ligation of the padlock probe was performed by PB-1 at 35° C. FIG. 6D provides the detection of transcripts in bone marrow, where ligation of the padlock probe was performed by PB-9 at 35° C. Tissue sections shown in FIGS. 6A and 6B were immobilized onto different lanes of the same flow cell. Similarly, tissue sections shown in FIGS. 6C and 6D were immobilized onto different lanes of the same flow cell. While both PB-1 and PB-9 enabled the detection of RNA molecules of interest in bone marrow, the use of PB-9 to ligate target-specific padlock probes resulted in reduced the frequency of off-tissue clusters; this was evidenced by the reduction of detected signal outside the tissue section when PB-9 was used for ligation at 30° C. and 35° C. Quantification of off-tissue clusters and off-tissue demuxed transcripts for sequencing experiments shown in FIGS. 6A and 6B are presented in FIGS. 6E and 6F. Use of PB-9 reduced the percentage of off-tissue clusters on bone marrow by about two-fold compared to the use of PB-1 as shown in FIG. 6E. The percentage of demuxed transcripts reduced by about 4-5-fold when PB-9 was used to ligate the target-specific padlock probes compared to when PB-1 was used.

FIG. 7 shows the gel obtained from SDS-PAGE, which depicts bands corresponding to PB-1 following incubation with MS(PEG)₄ and MS(PEG)₁₂ at 5×, 10×, or 15× molar ratio. Lane 1 shows the molecular weight ladder. Lanes 2 and 9 show the band of purified PB-9 incubated in buffer as a control. Lanes 3, 4, and 5 show bands of PB-9 incubated with MS(PEG)₄ at 5×, 10×, or 15× molar ratios, respectively. Lanes 6, 7, and 8 show bands of PB-9 incubated with MS(PEG)₁₂ at 5×, 10×, or 15× molar ratios, respectively.

FIG. 8 shows bands corresponding to PB-1_HT and PB-9 following incubation with MS(PEG)₂₄ at 0×, 1×, 2×, and 3× molar ratio. Lane 1 shows the molecular weight ladder. Lanes 2 and 9 show the bands of PB-1_HT and PB-9 incubated in buffer as a control. Lanes 3, 4, and 5 show bands of PB-1_HT incubated with MS(PEG)₂₄ at 1×, 2×, or 3× molar ratios, respectively. Lanes 6, 7, and 8 show bands of PB-9 incubated with MS(PEG)₂₄ at 1×, 2×, or 3× molar ratios, respectively.

FIGS. 9A-9C. 9A and 9B depict ligation reaction rates as fluorescence measured during the ligation reaction with pegylated ligase variants of PB-9 at 37° C. and 42° C. as a function of time, respectively. FIG. 9C provides the fluorescence measured for ligase variant PB-9 incubated with 1× (PEG)₂₄, 2× (PEG)₂₄, 5× (PEG)₂₄, and 5× (PEG)₁₂ following the incubation with DNA molecular beacon at 37° C. (shown on the left in lighter shade) and 42° C. (shown on the right in darker shade).

### DETAILED DESCRIPTION

The aspects and embodiments described herein relate to ligases. For example, provided herein are ligases and methods of detecting polynucleotide sequences, wherein detecting includes ligating a first polynucleotide sequence and a second polynucleotide sequence together using a ligase or variant described herein, wherein in embodiments, the ligase exhibits increased thermostability, improved ligation in situ, superior ligation activity at elevated temperatures, improved stability and resistance to aggregation relative to a control.

### I. Definitions

All patents, patent applications, articles and publications mentioned herein, both supra and infra, are hereby expressly incorporated herein by reference in their entireties.

Unless defined otherwise herein, all technical and scientific terms used herein have the same meaning as commonly understood by one of ordinary skill in the art to which this disclosure belongs. Various scientific dictionaries that include the terms included herein are well known and available to those in the art. Although any methods and materials similar or equivalent to those described herein find use in the practice or testing of the disclosure, some preferred methods and materials are described. Accordingly, the terms defined immediately below are more fully described by reference to the specification as a whole. It is to be understood that this disclosure is not limited to the particular methodology, protocols, and reagents described, as these may vary, depending upon the context in which they are used by those of skill in the art. The following definitions are provided to facilitate understanding of certain terms used frequently herein and are not meant to limit the scope of the present disclosure.

As used herein, the singular terms “a”, “an”, and “the” include the plural reference unless the context clearly indicates otherwise. Reference throughout this specification to, for example, “one embodiment”, “an embodiment”, “another embodiment”, “a particular embodiment”, “a related embodiment”, “a certain embodiment”, “an additional embodiment”, or “a further embodiment” or combinations thereof means that a particular feature, structure or characteristic described in connection with the embodiment is included in at least one embodiment of the present disclosure. Thus, the appearances of the foregoing phrases in various places throughout this specification are not necessarily all referring to the same embodiment. Furthermore, the particular features, structures, or characteristics may be combined in any suitable manner in one or more embodiments.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

As used herein, the term “about” means a range of values including the specified value, which a person of ordinary skill in the art would consider reasonably similar to the specified value. In embodiments, the term “about” means within a standard deviation using measurements generally acceptable in the art. In embodiments, about means a range extending to +/−10% of the specified value. In embodiments, about means the specified value.

Unless defined otherwise, technical and scientific terms used herein have the same meaning as commonly understood by a person of ordinary skill in the art. See, e.g., Singleton et al., DICTIONARY OF MICROBIOLOGY AND MOLECULAR BIOLOGY 2nd ed., J. Wiley & Sons (New York, NY 1994); Sambrook et al., MOLECULAR CLONING, A LABORATORY MANUAL, Cold Springs Harbor Press (Cold Springs Harbor, NY 1989). Any methods, devices and materials similar or equivalent to those described herein can be used in the practice of this invention. The following definitions are provided to facilitate understanding of certain terms used frequently herein and are not meant to limit the scope of the present disclosure.

“Nucleic acid” refers to nucleotides (e.g., deoxyribonucleotides or ribonucleotides) and polymers thereof in either single-, double- or multiple-stranded form, or complements thereof. The terms “polynucleotide,” “oligonucleotide,” “oligo” or the like refer, in the usual and customary sense, to a sequence of nucleotides. The term “nucleotide” refers, in the usual and customary sense, to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA, and hybrid molecules having mixtures of single and double stranded DNA and RNA with linear or circular framework. Non-limiting examples of polynucleotides include a gene, a gene fragment, an exon, an intron, intergenic DNA (including, without limitation, heterochromatic DNA), messenger RNA (mRNA), transfer RNA, ribosomal RNA, a ribozyme, cDNA, a recombinant polynucleotide, a branched polynucleotide, a plasmid, a vector, isolated DNA of a sequence, isolated RNA of a sequence, a nucleic acid probe, and a primer. Polynucleotides useful in the methods of the disclosure may comprise natural nucleic acid sequences and variants thereof, artificial nucleic acid sequences, or a combination of such sequences. As may be used herein, the terms “nucleic acid oligomer” and “oligonucleotide” are used interchangeably and are intended to include, but are not limited to, nucleic acids having a length of 200 nucleotides or less. In some embodiments, an oligonucleotide is a nucleic acid having a length of 2 to 200 nucleotides, 2 to 150 nucleotides, 5 to 150 nucleotides or 5 to 100 nucleotides. The terms “polynucleotide,” “oligonucleotide,” “oligo” or the like refer, in the usual and customary sense, to a linear sequence of nucleotides. Oligonucleotides are typically from about 5, 6, 7, 8, 9, 10, 12, 15, 25, 30, 40, 50 or more nucleotides in length, up to about 100 nucleotides in length. In some embodiments, an oligonucleotide is a primer configured for extension by a polymerase when the primer is annealed completely or partially to a complementary nucleic acid template. A primer is often a single stranded nucleic acid. In certain embodiments, a primer, or portion thereof, is substantially complementary to a portion of an adapter. In some embodiments, a primer has a length of 200 nucleotides or less. In certain embodiments, a primer has a length of 10 to 150 nucleotides, 15 to 150 nucleotides, 5 to 100 nucleotides, 5 to 50 nucleotides or 10 to 50 nucleotides. In some embodiments, an oligonucleotide may be immobilized to a solid support.

The term “duplex” in the context of polynucleotides refers, in the usual and customary sense, to double-strandedness. Nucleic acids can be linear or branched. For example, nucleic acids can be a linear chain of nucleotides or the nucleic acids can be branched, e.g., such that the nucleic acids comprise one or more arms or branches of nucleotides. Optionally, the branched nucleic acids are repetitively branched to form higher ordered structures such as dendrimers and the like. Different polynucleotides may have different three-dimensional structures, and may perform various functions, known or unknown. Nucleic acids, including e.g., nucleic acids with a phosphothioate backbone, can include one or more reactive moieties. As used herein, the term reactive moiety includes any group capable of reacting with another molecule, e.g., a nucleic acid or polypeptide through covalent, non-covalent or other interactions. By way of example, the nucleic acid can include an amino acid reactive moiety that reacts with an amino acid on a protein or polypeptide through a covalent, non-covalent or other interaction.

The term “base” and “nucleobase” as used herein refers to a purine or pyrimidine compound, or a derivative thereof, that may be a constituent of nucleic acid (i.e. DNA or RNA, or a derivative thereof). In embodiments, the base is a derivative of a naturally occurring DNA or RNA base (e.g., a base analogue). In embodiments, the base is a base-pairing base. In embodiments, the base pairs to a complementary base. In embodiments, the base is capable of forming at least one hydrogen bond with a complementary base (e.g., adenine hydrogen bonds with thymine, adenine hydrogen bonds with uracil, guanine pairs with cytosine). Non-limiting examples of a base includes cytosine or a derivative thereof (e.g., cytosine analogue), guanine or a derivative thereof (e.g., guanine analogue), adenine or a derivative thereof (e.g., adenine analogue), thymine or a derivative thereof (e.g., thymine analogue), uracil or a derivative thereof (e.g., uracil analogue), hypoxanthine or a derivative thereof (e.g., hypoxanthine analogue), xanthine or a derivative thereof (e.g., xanthine analogue), guanosine or a derivative thereof (e.g., 7-methylguanosine analogue), deaza-adenine or a derivative thereof (e.g., deaza-adenine analogue), deaza-guanine or a derivative thereof (e.g., deaza-guanine), deaza-hypoxanthine or a derivative thereof, 5,6-dihydrouracil or a derivative thereof (e.g., 5,6-dihydrouracil analogue), 5-methylcytosine or a derivative thereof (e.g., 5-methylcytosine analogue), or 5-hydroxymethylcytosine or a derivative thereof (e.g., 5-hydroxymethylcytosine analogue) moieties. In embodiments, the base is thymine, cytosine, uracil, adenine, guanine, hypoxanthine, xanthine, theobromine, caffeine, uric acid, or isoguanine. In embodiments, the base is

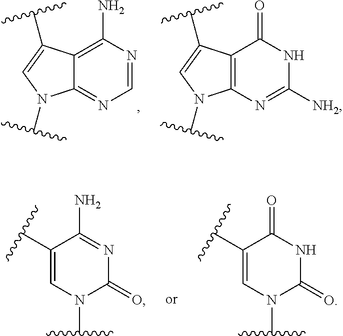 [SMILES: *c1cn(*)c2ncnc(N)c12 ; *c1cn(*)c2nc(N)[nH]c(=O)c12 ; *c1cn(*)c(=O)nc1N ; "or" ; *c1cn(*)c(=O)[nH]c1=O]

A polynucleotide is typically composed of a specific sequence of four nucleotide bases: adenine (A); cytosine (C); guanine (G); and thymine (T) (uracil (U) for thymine (T) when the polynucleotide is RNA. Thus, the term “polynucleotide sequence” is the alphabetical representation of a polynucleotide molecule; alternatively, the term may be applied to the polynucleotide molecule itself. This alphabetical representation can be input into databases in a computer having a central processing unit and used for bioinformatics applications such as functional genomics and homology searching. Polynucleotides may optionally include one or more non-standard nucleotide(s), nucleotide analog(s) and/or modified nucleotides.

The term “isolated”, when applied to a nucleic acid or protein, denotes that the nucleic acid or protein is essentially free of other cellular components with which it is associated in the natural state. It can be, for example, in a homogeneous state and may be in either a dry or an aqueous solution. Purity and homogeneity are typically determined using analytical chemistry techniques such as polyacrylamide gel electrophoresis or high performance liquid chromatography. A protein that is the predominant species present in a preparation is substantially purified.

The terms “analog” and “analogue” and “derivative” in reference to a chemical compound, refers to compounds having a structure similar to that of another one, but differing from it in respect of one or more different atoms, functional groups, or substructures that are replaced with one or more other atoms, functional groups, or substructures. In the context of a nucleotide useful in practicing the invention, a nucleotide analog refers to a compound that, like the nucleotide of which it is an analog, can be incorporated into a nucleic acid molecule (e.g., an extension product) by a suitable polymerase, for example, a DNA polymerase in the context of a dNTP analogue. The terms also encompass nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, include, without limitation, phosphodiester derivatives including, e.g., phosphoramidate, phosphorodiamidate, phosphorothioate (also known as phosphothioate having double bonded sulfur replacing oxygen in the phosphate), phosphorodithioate, phosphonocarboxylic acids, phosphonocarboxylates, phosphonoacetic acid, phosphonoformic acid, methyl phosphonate, boron phosphonate, or O-methylphosphoroamidite linkages (see Eckstein, OLIGONUCLEOTIDES AND ANALOGUES: A PRACTICAL APPROACH, Oxford University Press) as well as modifications to the nucleotide bases such as in 5-methyl cytidine or pseudouridine; and peptide nucleic acid backbones and linkages. Other analog nucleic acids include those with positive backbones; non-ionic backbones, modified sugars, and non-ribose backbones (e.g. phosphorodiamidate morpholino oligos or locked nucleic acids (LNA) as known in the art), including those described in U.S. Pat. Nos. 5,235,033 and 5,034,506, and Chapters 6 and 7, ASC Symposium Series 580, CARBOHYDRATE MODIFICATIONS IN ANTISENSE RESEARCH, Sanghui & Cook, eds. Nucleic acids containing one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made. In embodiments, the internucleotide linkages in DNA are phosphodiester, phosphodiester derivatives, or a combination of both.

As used herein, a “native” nucleotide is used in accordance with its plain and ordinary meaning and refers to a naturally occurring nucleotide that does not include an exogenous label (e.g., a fluorescent dye, or other label) or chemical modification such as may characterize a nucleotide analog. Examples of native nucleotides useful for carrying out procedures described herein include: dATP (2′-deoxyadenosine-5′-triphosphate); dGTP (2′-deoxyguanosine-5′-triphosphate); dCTP (2′-deoxycytidine-5′-triphosphate); dTTP (2′-deoxythymidine-5′-triphosphate); and dUTP (2′-deoxyuridine-5′-triphosphate).

The term “complement,” as used herein, refers to a nucleotide (e.g., RNA or DNA) or a sequence of nucleotides capable of base pairing with a complementary nucleotide or sequence of nucleotides. As described herein and commonly known in the art, the complementary (matching) nucleoside of adenosine is thymidine and the complementary (matching) nucleoside of guanosine is cytidine. Thus, a complement may include a sequence of nucleotides that base pair with corresponding complementary nucleotides of a second nucleic acid sequence. The nucleotides of a complement may match, partially or completely, the nucleotides of the second nucleic acid sequence. Where the nucleotides of the complement completely match each nucleotide of the second nucleic acid sequence, the complement forms base pairs with each nucleotide of the second nucleic acid sequence. Where the nucleotides of the complement partially match the nucleotides of the second nucleic acid sequence, only some of the nucleotides of the complement form base pairs with nucleotides of the second nucleic acid sequence. Examples of complementary sequences include coding and non-coding sequences, wherein the non-coding sequence contains complementary nucleotides to the coding sequence and thus forms the complement of the coding sequence. A further example of complementary sequences are sense and antisense sequences, wherein the sense sequence contains complementary nucleotides to the antisense sequence and thus forms the complement of the antisense sequence.

As described herein, the complementarity of sequences may be partial, in which only some of the nucleic acids match according to base pairing, or complete, where all the nucleic acids match according to base pairing. Thus, two sequences that are complementary to each other may have a specified percentage of nucleotides that are complementary (i.e., about 60% identity, preferably 65%, 70%, 75%, 80%, 85%, 90%, 91%, 92%, 93%, 94%, 95%, 96%, 97%, 98%, 99%, or higher complementarity over a specified region).

“DNA” refers to deoxyribonucleic acid, a polymer of deoxyribonucleotides (e.g., dATP, dCTP, dGTP, dTTP, dUTP, etc.) linked by phosphodiester bonds. DNA can be single-stranded (ssDNA) or double-stranded (dsDNA), and can include both single and double-stranded (or “duplex”) regions. “RNA” refers to ribonucleic acid, a polymer of ribonucleotides linked by phosphodiester bonds. RNA can be single-stranded (ssRNA) or double-stranded (dsRNA), and can include both single and double-stranded (or “duplex”) regions. Single-stranded DNA (or regions thereof) and ssRNA can, if sufficiently complementary, hybridize to form double-stranded DNA/RNA complexes (or regions).

The term “primer” refers to any nucleic acid molecule that may hybridize to a template and be bound by a DNA polymerase and extended in a template-directed process for nucleic acid synthesis. The primer may be a separate polynucleotide from the polynucleotide template, or both may be portions of the same polynucleotide (e.g., as in a hairpin structure having a 3′ end that is extended along another portion of the polynucleotide to extend a double-stranded portion of the hairpin). Primers (e.g., forward or reverse primers) may be attached to a solid support. A primer can be of any length depending on the particular technique it will be used for. For example, PCR primers are generally between 10 and 40 nucleotides in length. The length and complexity of the nucleic acid fixed onto the nucleic acid template may vary. In some embodiments, a primer has a length of 200 nucleotides or less. In certain embodiments, a primer has a length of 10 to 150 nucleotides, 15 to 150 nucleotides, 5 to 100 nucleotides, 5 to 50 nucleotides or 10 to 50 nucleotides. A primer typically has a length of 10 to 50 nucleotides. For example, a primer may have a length of 10 to 40, 10 to 30, 10 to 20, 25 to 50, 15 to 40, 15 to 30, 20 to 50, 20 to 40, or 20 to 30 nucleotides. In some embodiments, a primer has a length of 18 to 24 nucleotides. One of skill can adjust these factors to provide optimum hybridization and signal production for a given hybridization procedure. The primer permits the addition of a nucleotide residue thereto, or oligonucleotide or polynucleotide synthesis therefrom, under suitable conditions. In an embodiment, the primer is a DNA primer, i.e., a primer consisting of, or largely consisting of, deoxyribonucleotide residues. The primers are designed to have a sequence that is the complement of a region of template/target DNA to which the primer hybridizes. The addition of a nucleotide residue to the 3′ end of a primer by formation of a phosphodiester bond results in a DNA extension product. The addition of a nucleotide residue to the 3′ end of the DNA extension product by formation of a phosphodiester bond results in a further DNA extension product. In another embodiment the primer is an RNA primer. In embodiments, a primer is hybridized to a target polynucleotide. A “primer” is complementary to a polynucleotide template, and complexes by hydrogen bonding or hybridization with the template to give a primer/template complex for initiation of synthesis by a polymerase, which is extended by the addition of covalently bonded bases linked at its 3′ end complementary to the template in the process of DNA synthesis.

As used herein, the term “primer binding sequence” refers to a polynucleotide sequence that is complementary to at least a portion of a primer (e.g., a sequencing primer or an amplification primer). Primer binding sequences can be of any suitable length. In embodiments, a primer binding sequence is about or at least about 10, 15, 20, 25, 30, or more nucleotides in length. In embodiments, a primer binding sequence is 10-50, 15-30, or 20-25 nucleotides in length. The primer binding sequence may be selected such that the primer (e.g., sequencing primer) has the preferred characteristics to minimize secondary structure formation or minimize non-specific amplification, for example having a length of about 20-30 nucleotides; approximately 50% GC content, and a Tm of about 55° C. to about 65° C.

The term “DNA template” refers to any DNA molecule that may be bound by a DNA polymerase and utilized as a template for nucleic acid synthesis. In embodiments, the “DNA template” also refers to the DNA molecule that is subject to ligation by a ligase described herein.

The term “dATP analogue” refers to an analogue of deoxyadenosine triphosphate (dATP) that is a substrate for a DNA polymerase. The term “dCTP analogue” refers to an analogue of deoxycytidine triphosphate (dCTP) that is a substrate for a DNA polymerase. The term “dGTP analogue” refers to an analogue of deoxyguanosine triphosphate (dGTP) that is a substrate for a DNA polymerase. The term “dNTP analogue” refers to an analogue of deoxynucleoside triphosphate (dNTP) that is a substrate for a DNA polymerase. The term “dTTP analogue” refers to an analogue of deoxythymidine triphosphate (dUTP) that is a substrate for a DNA polymerase. The term “dUTP analogue” refers to an analogue of deoxyuridine triphosphate (dUTP) that is a substrate for a DNA polymerase.

The term “extendible” means, in the context of a nucleotide, primer, or extension product, that the 3′-OH group of the molecule is available and accessible to a DNA polymerase for extension or addition of nucleotides derived from dNTPs or dNTP analogues. “Incorporation” means joining of the modified nucleotide to the free 3′ hydroxyl group of a second nucleotide via formation of a phosphodiester linkage with the 5′ phosphate group of the modified nucleotide. The second nucleotide to which the modified nucleotide is joined will typically occur at the 3′ end of a polynucleotide chain.

The term “modified nucleotide” refers to nucleotide or nucleotide analogue modified in some manner. Typically, a nucleotide contains a single 5-carbon sugar moiety, a single nitrogenous base moiety and 1 to three phosphate moieties. In embodiments, a nucleotide can include a blocking moiety or a label moiety. A blocking moiety (e.g., a reversible terminator moiety) on a nucleotide prevents formation of a covalent bond between the 3′ hydroxyl moiety of the nucleotide and the 5′ phosphate of another nucleotide. A blocking moiety on a nucleotide can be reversible (i.e., a reversible terminator), whereby the blocking moiety can be removed or modified to allow the 3′ hydroxyl to form a covalent bond with the 5′ phosphate of another nucleotide. A blocking moiety can be effectively irreversible under particular conditions used in a method set forth herein. A label moiety of a nucleotide can be any moiety that allows the nucleotide to be detected, for example, using a spectroscopic method. Exemplary label moieties are fluorescent labels, mass labels, chemiluminescent labels, electrochemical labels, detectable labels and the like. One or more of the above moieties can be absent from a nucleotide used in the methods and compositions set forth herein. For example, a nucleotide can lack a label moiety or a blocking moiety or both.

A “removable” group, e.g., a label or a blocking group or protecting group, refers to a chemical group that can be removed from a dNTP analogue such that a DNA polymerase can extend the nucleic acid (e.g., a primer or extension product) by the incorporation of at least one additional nucleotide. Removal may be by any suitable method, including enzymatic, chemical, or photolytic cleavage. Removal of a removable group, e.g., a blocking group, does not require that the entire removable group be removed, only that a sufficient portion of it be removed such that a DNA polymerase can extend a nucleic acid by incorporation of at least one additional nucleotide using a dNTP of dNTP analogue.

“Reversible blocking groups” or “reversible terminators” include a blocking moiety located, for example, at the 3′ position of the nucleotide and may be a chemically cleavable moiety such as an allyl group, an azidomethyl group or a methoxymethyl group, or may be an enzymatically cleavable group such as a phosphate ester. Suitable nucleotide blocking moieties are described in applications WO 2004/018497, U.S. Pat. Nos. 7,057,026, 7,541,444, WO 96/07669, U.S. Pat. Nos. 5,763,594, 5,808,045, 5,872,244 and 6,232,465 the contents of which are incorporated herein by reference in their entirety. The nucleotides may be labelled or unlabeled. They may be modified with reversible terminators useful in methods provided herein and may be 3′-O-blocked reversible or 3′-unblocked reversible terminators. In nucleotides with 3′-O-blocked reversible terminators, the blocking group —OR [reversible terminating (capping) group] is linked to the oxygen atom of the 3′-OH of the pentose, while the label is linked to the base, which acts as a reporter and can be cleaved. The 3′-O-blocked reversible terminators are known in the art, and may be, for instance, a 3′-ONH₂ reversible terminator, a 3′-O-allyl reversible terminator, or a 3′-O-azidomethyl reversible terminator.

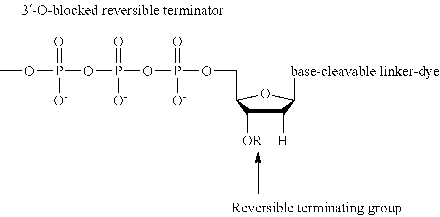

The term “non-covalent linker” is used in accordance with its ordinary meaning and refers to a divalent moiety which includes at least two molecules that are not covalently linked to each other but are capable of interacting with each other via a non-covalent bond (e.g. electrostatic interactions (e.g. ionic bond, hydrogen bond, halogen bond) or van der Waals interactions (e.g. dipole-dipole, dipole-induced dipole, London dispersion). In embodiments, the non-covalent linker is the result of two molecules that are not covalently linked to each other that interact with each other via a non-covalent bond.

The terms “cleavable linker” or “cleavable moiety” as used herein refers to a divalent or monovalent, respectively, moiety which is capable of being separated (e.g., detached, split, disconnected, hydrolyzed, a stable bond within the moiety is broken) into distinct entities. A cleavable linker is cleavable (e.g., specifically cleavable) in response to external stimuli (e.g., enzymes, nucleophilic/basic reagents, reducing agents, photo-irradiation, electrophilic/acidic reagents, organometallic and metal reagents, or oxidizing reagents). A chemically cleavable linker refers to a linker which is capable of being split in response to the presence of a chemical (e.g., acid, base, oxidizing agent, reducing agent, Pd(0), tris-(2-carboxyethyl)phosphine, dilute nitrous acid, fluoride, tris(3-hydroxypropyl)phosphine), sodium dithionite (Na₂S₂O₄), hydrazine (N₂H₄)). A chemically cleavable linker is non-enzymatically cleavable. In embodiments, the cleavable linker is cleaved by contacting the cleavable linker with a cleaving agent. In embodiments, the cleaving agent is sodium dithionite (Na₂S₂O₄), weak acid, hydrazine (N₂H₄), Pd(0), or light-irradiation (e.g., ultraviolet radiation).

The term “orthogonal detectable label” or “orthogonal detectable moiety” as used herein refer to a detectable label (e.g. fluorescent dye or detectable dye) that is capable of being detected and identified (e.g., by use of a detection means (e.g., emission wavelength, physical characteristic measurement)) in a mixture or a panel (collection of separate samples) of two or more different detectable labels. For example, two different detectable labels that are fluorescent dyes are both orthogonal detectable labels when a panel of the two different fluorescent dyes is subjected to a wavelength of light that is absorbed by one fluorescent dye but not the other and results in emission of light from the fluorescent dye that absorbed the light but not the other fluorescent dye. Orthogonal detectable labels may be separately identified by different absorbance or emission intensities of the orthogonal detectable labels compared to each other and not only be the absolute presence of absence of a signal. An example of a set of four orthogonal detectable labels is the set of Rox™-Labeled Tetrazine, Alexa Fluor® 488-Labeled SHA, Cy®5-Labeled Streptavidin, and R6G-Labeled Dibenzocyclooctyne. ROX™ is a trademark of Applera Corporation. Alexa Fluor® is a trademark of Life Technologies Corporation. Cy® is a trademark of Cytiva.

A “detectable agent” or “detectable compound” or “detectable label” or “detectable moiety” is a composition detectable by spectroscopic, photochemical, biochemical, immunochemical, chemical, magnetic resonance imaging, or other physical means. For example, detectable agents include fluorophores (e.g. fluorescent dyes), modified oligonucleotides (e.g., moieties described in PCT/US2015/022063, which is incorporated herein by reference), electron-dense reagents, enzymes (e.g., as commonly used in an ELISA), biotin, digoxigenin, paramagnetic molecules, paramagnetic nanoparticles, ultrasmall superparamagnetic iron oxide (“USPIO”) nanoparticles, USPIO nanoparticle aggregates, superparamagnetic iron oxide (“SPIO”) nanoparticles, SPIO nanoparticle aggregates, monochrystalline iron oxide nanoparticles, monochrystalline iron oxide, nanoparticle contrast agents, liposomes or other delivery vehicles containing Gadolinium chelate (“Gd-chelate”) molecules, Gadolinium, radioisotopes, radionuclides (e.g. carbon-11, nitrogen-13, oxygen-15, fluorine-18, rubidium-82), fluorodeoxyglucose (e.g. fluorine-18 labeled), any gamma ray emitting radionuclides, positron-emitting radionuclide, radiolabeled glucose, radiolabeled water, radiolabeled ammonia, biocolloids, microbubbles (e.g. including microbubble shells including albumin, galactose, lipid, and/or polymers; microbubble gas core including air, heavy gas(es), perfluorcarbon, nitrogen, octafluoropropane, perflexane lipid microsphere, perflutren, etc.), iodinated contrast agents (e.g. iohexol, iodixanol, ioversol, iopamidol, ioxilan, iopromide, diatrizoate, metrizoate, ioxaglate), barium sulfate, thorium dioxide, gold, gold nanoparticles, gold nanoparticle aggregates, fluorophores, two-photon fluorophores, or haptens and proteins or other entities which can be made detectable, e.g., by incorporating a radiolabel into a peptide or antibody specifically reactive with a target peptide. Examples of detectable agents include imaging agents, including fluorescent and luminescent substances, including, but not limited to, a variety of organic or inorganic small molecules commonly referred to as “dyes,” “labels,” or “indicators.” Examples include fluorescein, rhodamine, acridine dyes, Alexa Fluor® dyes, and cyanine dyes. In embodiments, the detectable moiety is a fluorescent molecule (e.g., acridine dye, cyanine, dye, fluorine dye, oxazine dye, phenanthridine dye, or rhodamine dye). In embodiments, the detectable moiety is a fluorescent molecule (e.g., acridine dye, cyanine, dye, fluorine dye, oxazine dye, phenanthridine dye, or rhodamine dye). In embodiments, the detectable moiety is a fluorescein isothiocyanate moiety, tetramethylrhodamine-5-(and 6)-isothiocyanate moiety, Cy®2 moiety, Cy®3 moiety, Cy®5 moiety, Cy®7 moiety, 4′,6-diamidino-2-phenylindole moiety, Hoechst 33258 moiety, Hoechst 33342 moiety, Hoechst 34580 moiety, propidium-iodide moiety, or acridine orange moiety. In embodiments, the detectable label is a fluorescent dye. In embodiments, the detectable label is a fluorescent dye capable of exchanging energy with another fluorescent dye (e.g., fluorescence resonance energy transfer (FRET) chromophores).

A “cleavable site” or “scissile linkage” in the context of a polynucleotide is a site which allows controlled cleavage of the polynucleotide strand (e.g., the linker, the primer, or the polynucleotide) by chemical, enzymatic, or photochemical means known in the art and described herein. A scissile site may refer to the linkage of a nucleotide between two other nucleotides in a nucleotide strand (i.e., an internucleosidic linkage). In embodiments, the scissile linkage can be located at any position within the one or more nucleic acid molecules, including at or near a terminal end (e.g., the 3′ end of an oligonucleotide) or in an interior portion of the one or more nucleic acid molecules. In embodiments, conditions suitable for separating a scissile linkage include a modulating the pH and/or the temperature. In embodiments, a scissile site can include at least one acid-labile linkage. For example, an acid-labile linkage may include a phosphoramidate linkage. In embodiments, a phosphoramidate linkage can be hydrolysable under acidic conditions, including mild acidic conditions such as trifluoroacetic acid and a suitable temperature (e.g., 30° C.), or other conditions known in the art, for example Matthias Mag, et al Tetrahedron Letters, Volume 33, Issue 48, 1992, 7319-7322. In embodiments, the scissile site can include at least one photolabile internucleosidic linkage (e.g., o-nitrobenzyl linkages, as described in Walker et al, J. Am. Chem. Soc. 1988, 110, 21, 7170-7177), such as o-nitrobenzyloxymethyl or p-nitrobenzyloxymethyl group(s). In embodiments, the scissile site includes at least one uracil nucleobase. In embodiments, a uracil nucleobase can be cleaved with a uracil DNA glycosylase (UDG) or Formamidopyrimidine DNA Glycosylase Fpg. In embodiments, the scissile linkage site includes a sequence-specific nicking site having a nucleotide sequence that is recognized and nicked by a nicking endonuclease enzyme or a uracil DNA glycosylase.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an α carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. The terms “non-naturally occurring amino acid” and “unnatural amino acid” refer to amino acid analogs, synthetic amino acids, and amino acid mimetics, which are not found in nature.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The terms “polypeptide,” “peptide” and “protein” are used interchangeably herein to refer to a polymer of amino acid residues, wherein the polymer may in embodiments be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers. A “fusion protein” refers to a chimeric protein encoding two or more separate protein sequences that are recombinantly expressed as a single moiety.

“Conservatively modified variants” applies to both amino acid and nucleic acid sequences. With respect to particular nucleic acid sequences, “conservatively modified variants” refers to those nucleic acids that encode identical or essentially identical amino acid sequences. Because of the degeneracy of the genetic code, a number of nucleic acid sequences will encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein, which encodes a polypeptide, also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid that encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the disclosure.

The following groups each contain amino acids that are conservative substitutions for one another: 1) Non-polar—Alanine (A), Leucine (L), Isoleucine (I), Valine (V), Glycine (G), Methionine (M); 2) Aliphatic—Alanine (A), Leucine (L), Isoleucine (I), Valine (V); 3) Acidic—Aspartic acid (D), Glutamic acid (E); 4) Polar—Asparagine (N), Glutamine (Q); Serine (S), Threonine (T); 5) Basic—Arginine (R), Lysine (K); 7) Aromatic—Phenylalanine (F), Tyrosine (Y), Tryptophan (W), Histidine (H); 8) Other—Cysteine (C) and Proline (P).

“Percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity. Percent identity often refers to the percentage of matching positions of two sequences for a contiguous section of positions, wherein the two sequences are aligned in such a way to maximize matching positions and minimize gaps of non-matching positions. In some embodiments, alignments are conducted wherein there are no gaps between the two sequences. In some instances, the alignment results in less than 5% gaps, less than 3% gaps, or less than 1% gaps. Additional methods of sequence comparison or alignment are also consistent with the disclosure.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST® or BLAST® 2.0 sequence comparison algorithm with default parameters described below, or by manual alignment and visual inspection (see, e.g., NCBI web site www.ncbi.nlm.nih.gov/BLAST/or the like). Such sequences are then said to be “substantially identical.” This definition also refers to, or may be applied to, the complement of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length. As used herein, percent (%) amino acid sequence identity is defined as the percentage of amino acids in a candidate sequence that is identical to the amino acids in a reference sequence, after aligning the sequences and introducing gaps, if necessary, to achieve the maximum percent sequence identity. Alignment for purposes of determining percent sequence identity can be achieved in various ways that are within the level of skill in the art, for instance, using publicly available computer software such as BLAST®, BLAST®-2, ALIGN, ALIGN-2 or Megalign (DNASTAR) software. Appropriate parameters for measuring alignment, including any algorithms needed to achieve maximal alignment over the full length of the sequences being compared can be determined by known methods.

For sequence comparisons, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 10 to 700, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, WI), or by manual alignment and visual inspection (see, e.g., Current Protocols in Molecular Biology (Ausubel et al., eds. 1995 supplement)).

An amino acid or nucleotide base “position” is denoted by a number that sequentially identifies each amino acid (or nucleotide base) in the reference sequence based on its position relative to the N-terminus (or 5′-end). Due to deletions, insertions, truncations, fusions, and the like that must be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

The terms “position”, “numbered with reference to” or “corresponding to,” when used in the context of the numbering of a given amino acid or polynucleotide sequence, refer to the numbering of the residues of a specified reference sequence when the given amino acid or polynucleotide sequence is compared to the reference sequence. Similarly, the term “functionally equivalent to” in relation to an amino acid position refers to an amino acid residue in a protein that corresponds to a particular amino acid in a reference sequence. An amino acid “corresponds” to a given residue when it occupies the same essential structural position within the protein as the given residue. One skilled in the art will immediately recognize the identity and location of residues corresponding to a specific position in a protein (e.g., ligase) in other proteins with different numbering systems. For example, by performing a simple sequence alignment with a protein (e.g., ligase) the identity and location of residues corresponding to specific positions of the protein are identified in other protein sequences aligning to the protein. For example, a selected residue in a selected protein corresponds to cysteine at position 22 when the selected residue occupies the same essential spatial or other structural relationship as a cysteine at position 22. In some embodiments, where a selected protein is aligned for maximum homology with a protein, the position in the aligned selected protein aligning with cysteine 22 is said to correspond to cysteine 22. Instead of a primary sequence alignment, a three-dimensional structural alignment can also be used, e.g., where the structure of the selected protein is aligned for maximum correspondence with the cysteine at position 22, and the overall structures compared. In this case, an amino acid that occupies the same essential position as cysteine 22 in the structural model is said to correspond to the cysteine 22 residue. Sequence alignments may be compiled using any of the standard alignment tools known in the art, such as for example BLAST® and DIAMOND (Buchfink et al. Nat Methods 12, 59-60 (2015)), and the like.

The term “DNA polymerase” and “nucleic acid polymerase” are used in accordance with their plain ordinary meaning and refer to enzymes capable of synthesizing nucleic acid molecules from nucleotides (e.g., deoxyribonucleotides). Typically, a DNA polymerase adds nucleotides to the 3′ end of a DNA strand one nucleotide at a time.

The term “exonuclease activity” is used in accordance with its ordinary meaning in the art, and refers to the removal of a nucleotide from a nucleic acid by a DNA polymerase. For example, during polymerization, nucleotides are added to the 3′ end of the primer strand. Occasionally a DNA polymerase incorporates an incorrect nucleotide to the 3′-OH terminus of the primer strand, wherein the incorrect nucleotide cannot form a hydrogen bond to the corresponding base in the template strand. Such a nucleotide, added in error, is removed from the primer as a result of the 3′ to 5′ exonuclease activity of the DNA polymerase. In embodiments, exonuclease activity may be referred to as “proofreading.” When referring to 3′-5′ exonuclease activity, it is understood that the DNA polymerase facilitates a hydrolyzing reaction that breaks phosphodiester bonds at the 3′ end of a polynucleotide chain to excise the nucleotide, thereby releasing deoxyribonucleoside 5′-monophosphates one after another. One having skill in the art understands that an enzyme having 3′-5′ exonuclease activity does not cleave DNA strands without terminal 3′-OH moieties. In embodiments, 3′-5′ exonuclease activity refers to the successive removal of nucleotides in single-stranded DNA in a 3′→5′ direction, releasing deoxyribonucleoside 5′-monophosphates one after another. Methods for quantifying exonuclease activity are known in the art, see for example Southworth et al, PNAS Vol 93, 8281-8285 (1996).

The terms “measure”, “measuring”, “measurement” and the like refer not only to quantitative measurement of a particular variable, but also to qualitative and semi-quantitative measurements. Accordingly, “measurement” also includes detection, meaning that merely detecting a change, without quantification, constitutes measurement.

A “polymerase-template complex” refers to a functional complex between a DNA polymerase and a DNA primer-template molecule (e.g., nucleic acid). In embodiments, the polymerase is non-covalently bound to a nucleic acid primer and the template nucleic acid molecule.

The terms “sequencing”, “sequence determination”, “determining a nucleotide sequence”, and the like include determination of partial as well as full sequence information of the polynucleotide being sequenced. That is, the term includes sequence comparisons, fingerprinting, and like levels of information about a target polynucleotide, as well as the express identification and ordering of nucleotides in a target polynucleotide. The term also includes the determination of the identification, ordering, and locations of one, two, or three of the four types of nucleotides within a target polynucleotide.

The term “sequencing reaction mixture” refers to an aqueous mixture that contains the reagents necessary to allow a dNTP or dNTP analogue to add a nucleotide to a DNA strand by a DNA polymerase. Exemplary mixtures include buffers (e.g., saline-sodium citrate (SSC), tris(hydroxymethyl)aminomethane or “Tris”), salts (e.g., KCl or (NH₄)₂SO₄)), nucleotides, polymerases, cleaving agent (e.g., tri-n-butyl-phosphine, triphenyl phosphine and its sulfonated versions (i.e., tris(3-sulfophenyl)-phosphine, TPPTS), and tri(carboxyethyl)phosphine (TCEP) and its salts, cleaving agent scavenger compounds (e.g., 2′-Dithiobisethanamine or 11-Azido-3,6,9-trioxaundecane-1-amine), detergents and/or crowding agents or stabilizers (e.g., PEG, Tween®, BSA). Tween® is a registered trademark of Croda International PLC.

As used herein, the terms “solid support” and “substrate” and “substrate surface” and “solid surface” refers to discrete surfaces that are solid or semi-solid. A solid support may encompass any type of solid, porous, or hollow sphere, ball, cylinder, or other similar configuration composed of plastic, ceramic, metal, or polymeric material (e.g., hydrogel) onto which a nucleic acid may be immobilized (e.g., covalently or non-covalently). A solid support may include a discrete particle that may be spherical (e.g., microspheres) or have a non-spherical or irregular shape, such as cubic, cuboid, pyramidal, cylindrical, conical, oblong, or disc-shaped, and the like. A bead can be non-spherical in shape. A solid support may be used interchangeably with the term “bead.” A solid support may further include a polymer or hydrogel on the surface to which the primers are attached. Exemplary solid supports include, but are not limited to, glass and modified or functionalized glass, plastics (including acrylics, polystyrene and copolymers of styrene and other materials, polypropylene, polyethylene, polybutylene, polyurethanes, Teflon™ cyclic olefin copolymers, polyimides etc.), nylon, ceramics, resins, Zeonor®, silica or silica-based materials including silicon and modified silicon, carbon, metals, inorganic glasses, optical fiber bundles, photopatternable dry film resists, UV-cured adhesives and polymers. Particularly useful solid supports for some embodiments have at least one surface located on a microplate. Particularly useful solid supports for some embodiments have at least one surface located on a microplate within a flow cell. Solid surfaces can also be varied in their shape depending on the application in a method described herein. For example, a solid surface useful herein can be planar, or contain regions which are concave or convex. In embodiments, the geometry of the concave or convex regions (e.g., wells) of the solid surface conform to the size and shape of a substantially circular particle to maximize the contact between the particle. In embodiments, the wells of an array are randomly located such that nearest neighbor wells have random spacing between each other. Alternatively, in embodiments the spacing between the wells can be ordered, for example, forming a regular pattern. The term solid substrate is encompassing of a substrate (e.g., a microplate or flow cell) having a surface including a polymer coating covalently attached thereto.

Broadly speaking, for nucleic acid sequencing applications, a flow cell may be considered a reaction chamber that contains one or more nucleic acid templates tethered to a solid support, to which nucleotides and ancillary reagents are iteratively applied and washed away. The flow cell allows for imaging of the sites at which the nucleic acids are bound, and resulting image data is used for the desired analysis. The latest commercial sequencing instruments use flow cells and massive parallelization to increase sequencing capacity.

In embodiments, the solid substrate is a flow cell. The term “flow cell” as used herein refers to a chamber including a solid surface across which one or more fluid reagents can be flowed. Examples of flow cells and related fluidic systems and detection platforms that can be readily used in the methods of the present disclosure are described, for example, in Bentley et al., Nature 456:53-59 (2008). In certain embodiments a substrate includes a surface (e.g., a surface of a flow cell, a surface of a tube, a surface of a chip), for example a metal surface (e.g., steel, gold, silver, aluminum, silicon and copper). In embodiments a substrate (e.g., a substrate surface) is coated and/or includes functional groups and/or inert materials. In certain embodiments a substrate includes a bead, a chip, a capillary, a plate, a membrane, a wafer (e.g., silicon wafers), a comb, or a pin for example. In some embodiments a substrate includes a bead and/or a nanoparticle. A substrate can be made of a suitable material, non-limiting examples of which include a plastic or a suitable polymer (e.g., polycarbonate, poly(vinyl alcohol), poly(divinylbenzene), polystyrene, polyamide, polyester, polyvinylidene difluoride (PVDF), polyethylene, polyurethane, polypropylene, and the like), borosilicate, glass, nylon, Wang resin, Merrifield resin, metal (e.g., iron, a metal alloy, sepharose, agarose, polyacrylamide, dextran, cellulose and the like or combinations thereof. In embodiments a substrate includes a magnetic material (e.g., iron, nickel, cobalt, platinum, aluminum, and the like). In embodiments a substrate includes a magnetic bead (e.g., DYNABEADS®, hematite, AMPure XP). Magnets can be used to purify and/or capture nucleic acids bound to certain substrates (e.g., substrates including a metal or magnetic material). The flow cell is typically a glass slide containing small fluidic channels (e.g., a glass slide 75 mm×25 mm×1 mm having one or more channels), through which sequencing solutions (e.g., polymerases, nucleotides, and buffers) may traverse. Though typically glass, suitable flow cell materials may include polymeric materials, plastics, silicon, quartz (fused silica), Borofloat® glass, silica, silica-based materials, carbon, metals, an optical fiber or optical fiber bundles, sapphire, or plastic materials such as COCs and epoxies. The particular material can be selected based on properties desired for a particular use. For example, materials that are transparent to a desired wavelength of radiation are useful for analytical techniques that will utilize radiation of the desired wavelength. Conversely, it may be desirable to select a material that does not pass radiation of a certain wavelength (e.g., being opaque, absorptive, or reflective). In embodiments, the material of the flow cell is selected due to the ability to conduct thermal energy. In embodiments, a flow cell includes inlet and outlet ports and a flow channel extending there between.

As used herein, the term “channel” refers to a passage in or on a substrate material that directs the flow of a fluid. A channel may run along the surface of a substrate, or may run through the substrate between openings in the substrate. A channel can have a cross section that is partially or fully surrounded by substrate material (e.g., a fluid impermeable substrate material). For example, a partially surrounded cross section can be a groove, trough, furrow or gutter that inhibits lateral flow of a fluid. The transverse cross section of an open channel can be, for example, U-shaped, V-shaped, curved, angular, polygonal, or hyperbolic. A channel can have a fully surrounded cross section such as a tunnel, tube, or pipe. A fully surrounded channel can have a rounded, circular, elliptical, square, rectangular, or polygonal cross section. In particular embodiments, a channel can be located in a flow cell, for example, being embedded within the flow cell. A channel in a flow cell can include one or more windows that are transparent to light in a particular region of the wavelength spectrum. In embodiments, the channel is filled by the one or more polymers, and flow through the channel (e.g., as in a sample fluid) is directed through the polymer in the channel. In embodiments, the tissue is in a channel of a flow cell.

The term “array” as used herein, refers to a container (e.g., a multiwell container, reaction vessel, or flow cell) including a plurality of features (e.g., wells). For example, an array may include a container with a plurality of wells. In embodiments, the array is a microplate. In embodiments, the array is a flow cell.

The term “microplate,” “microtiter plate,” or “multiwell plate” as used herein, refers to a substrate including a surface, the surface including a plurality of chambers or wells separated from each other by interstitial regions on the surface. In embodiments, the microplate has dimensions as provided and described by American National Standards Institute (ANSI) and Society for Laboratory Automation And Screening (SLAS); for example the tolerances and dimensions set forth in ANSI SLAS 1-2004 (R2012); ANSI SLAS 2-2004 (R2012); ANSI SLAS 3-2004 (R2012); ANSI SLAS 4-2004 (R2012); and ANSI SLAS 6-2012, which are incorporated herein by reference. The dimensions of the microplate as described herein and the arrangement of the reaction chambers may be compatible with an established format for automated laboratory equipment. In embodiments, the device described herein provides methods for high-throughput screening. High-throughput screening (HTS) refers to a process that uses a combination of modern robotics, data processing and control software, liquid handling devices, and/or sensitive detectors, to efficiently process a large amount of (e.g., thousands, hundreds of thousands, or millions) samples in biochemical, genetic, or pharmacological experiments, either in parallel or in sequence, within a reasonably short period of time (e.g., days). Preferably, the process is amenable to automation, such as robotic simultaneous handling of 96 samples, 384 samples, 1536 samples or more. A typical HTS robot tests up to 100,000 to a few hundred thousand compounds per day. The samples are often in small volumes, such as no more than 1 mL, 500 μL, 200 μL, 100 μL, 50 μL or less. Through this process, one can rapidly identify active compounds, small molecules, antibodies, proteins, or polynucleotides in a cell.

The reaction chambers may be provided as wells, for example an array or microplate may contain 2, 4, 6, 12, 24, 48, 96, 384, or 1536 sample wells. In embodiments, the 96 and 384 wells are arranged in a 2:3 rectangular matrix. In embodiments, the 24 wells are arranged in a 3:8 rectangular matrix. In embodiments, the 48 wells are arranged in a 3:4 rectangular matrix. In embodiments, the reaction chamber is a microscope slide (e.g., a glass slide about 75 mm by about 25 mm). In embodiments the slide is a concavity slide (e.g., the slide includes a depression). In embodiments, the slide includes a coating for enhanced cell adhesion (e.g., poly-L-lysine, silanes, carbon nanotubes, polymers, epoxy resins, or gold). In embodiments, the microplate is about 5 inches by about 3.33 inches, and includes a plurality of 5 mm diameter wells. In embodiments, the microplate is about 5 inches by about 3.33 inches, and includes a plurality of 6 mm diameter wells. In embodiments, the microplate is about 5 inches by about 3.33 inches, and includes a plurality of 7 mm diameter wells. In embodiments, the microplate is about 5 inches by about 3.33 inches, and includes a plurality of 7.5 mm diameter wells. In embodiments, the microplate is 5 inches by 3.33 inches, and includes a plurality of 7.5 mm diameter wells. In embodiments, the microplate is about 5 inches by about 3.33 inches, and includes a plurality of 8 mm diameter wells. In embodiments, the microplate is a flat glass or plastic tray in which an array of wells are formed, wherein each well can hold between from a few microliters to hundreds of microliters of fluid reagents and samples.

The term “species”, when used in the context of describing a particular compound or molecule species, refers to a population of chemically indistinct molecules. When used in the context of taxonomy, “species” is the basic unit of classification and a taxonomic rank.

The term “expression” includes any step involved in the production of the polypeptide including, but not limited to, transcription, post-transcriptional modification, translation, post-translational modification, and secretion. Expression can be detected using conventional techniques for detecting protein (e.g., ELISA, Western blotting, flow cytometry, immunofluorescence, immunohistochemistry, etc.).

A “cell” as used herein, refers to a cell carrying out metabolic or other function sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaryotic cells. Prokaryotic cells include but are not limited to bacteria. Eukaryotic cells include but are not limited to yeast cells and cells derived from plants and animals, for example mammalian, insect (e.g., spodoptera) and human cells.

“Control” or “control experiment” is used in accordance with its plain ordinary meaning and refers to an experiment in which the subjects (e.g., enzymes) or reagents of the experiment are treated as in a parallel experiment except for omission of a procedure, reagent, or variable of the experiment (e.g., a ligase not having one or more mutations relative to the polymerase being tested). In some instances, the control is used as a standard of comparison in evaluating experimental effects. In some embodiments, a control is the measurement of the activity of a protein in the absence of a mutation as described herein (including embodiments and examples). “Control ligase” is defined herein as the ligase against which the activity of the altered ligase is compared. Unless otherwise stated, by “wild type” it is generally meant that the ligase comprises its natural amino acid sequence, as it would be found in nature. The invention is not limited to merely a comparison of activity of the ligase as described herein against the wild type. Many ligases exist whose amino acid sequence has been modified (e.g., by amino acid substitution mutations) and which can prove to be a suitable control for use in assessing the ligation efficiencies of the ligases as described herein. The control ligase can, therefore, include any known ligase, including mutant ligases known in the art. The activity of the chosen “control” ligase with respect to the ligation of single-stranded DNA polynucleotides may be determined by a ligation activity assay as described infra. In embodiments, the control includes performing the experiment with a wild type ligase.

The term “modulate” is used in accordance with its plain ordinary meaning and refers to the act of changing or varying one or more properties. “Modulation” refers to the process of changing or varying one or more properties.

The term “kit” is used in accordance with its plain ordinary meaning and refers to any delivery system for delivering materials or reagents for carrying out a method of the invention. Such delivery systems include systems that allow for the storage, transport, or delivery of reaction reagents (e.g., nucleotides, enzymes, nucleic acid templates, etc. in the appropriate containers) and/or supporting materials (e.g., buffers, written instructions for performing the reaction, etc.) from one location to another location. For example, kits include one or more enclosures (e.g., boxes) containing the relevant reaction reagents and/or supporting materials. Such contents may be delivered to the intended recipient together or separately. For example, a first container may contain an enzyme, while a second container contains nucleotides. In embodiments, the kit includes vessels containing one or more enzymes, primers, adaptors, or other reagents as described herein. Vessels may include any structure capable of supporting or containing a liquid or solid material and may include tubes, vials, jars, containers, tips, etc. In embodiments, a wall of a vessel may permit the transmission of light through the wall. In embodiments, the vessel may be optically clear. The kit may include the enzyme and/or nucleotides in a buffer. In embodiments, the buffer includes an acetate buffer, 3-(N-morpholino)propanesulfonic acid (MOPS) buffer, N-(2-Acetamido)-2-aminoethanesulfonic acid (ACES) buffer, phosphate-buffered saline (PBS) buffer, 4-(2-hydroxyethyl)-1-piperazineethanesulfonic acid (HEPES) buffer, N-(1,1-Dimethyl-2-hydroxyethyl)-3-amino-2-hydroxypropanesulfonic acid (AMPSO) buffer, borate buffer (e.g., borate buffered saline, sodium borate buffer, boric acid buffer), 2-Amino-2-methyl-1,3-propanediol (AMPD) buffer, N-cyclohexyl-2-hydroxyl-3-aminopropanesulfonic acid (CAPSO) buffer, 2-Amino-2-methyl-1-propanol (AMP) buffer, 4-(Cyclohexylamino)-1-butanesulfonic acid (CABS) buffer, glycine-NaOH buffer, N-Cyclohexyl-2-aminoethanesulfonic acid (CHES) buffer, tris(hydroxymethyl)aminomethane (Tris) buffer, or a N-cyclohexyl-3-aminopropanesulfonic acid (CAPS) buffer. In embodiments, the buffer is a borate buffer. In embodiments, the buffer is a CHES buffer.

Where a range of values is provided, it is understood that each intervening value, to the tenth of the unit of the lower limit unless the context clearly indicates otherwise, between the upper and lower limit of that range, and any other stated or unstated intervening value in, or smaller range of values within, that stated range is encompassed within the invention. The upper and lower limits of any such smaller range (within a more broadly recited range) may independently be included in the smaller ranges, or as particular values themselves, and are also encompassed within the invention, subject to any specifically excluded limit in the stated range. Where the stated range includes one or both of the limits, ranges excluding either or both of those included limits are also included in the invention.

The phrase “stringent hybridization conditions” refers to conditions under which a primer will hybridize to its target subsequence, typically in a complex mixture of nucleic acids, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, stringent conditions are selected to be about 5-10° C. lower than the thermal melting point (Tₘ) for the specific sequence at a defined ionic strength pH. The Tₘ is the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions may also be achieved with the addition of destabilizing agents such as formamide. For selective or specific hybridization, a positive signal is at least two times background, preferably 10 times background hybridization. Exemplary stringent hybridization conditions can be as following: 50% formamide, 5×SSC, and 1% SDS, incubating at 42° C., or 5×SSC, 1% SDS, incubating at 65° C., with wash in 0.2×SSC, and 0.1% SDS at 65° C.

The term “isolated” means altered or removed from the natural state. For example, a nucleic acid or a polypeptide naturally present in a living animal is not isolated, but the same nucleic acid or polypeptide partially or completely separated from the coexisting materials of its natural state is isolated. An isolated nucleic acid or protein can exist in substantially purified form, or can exist in a non-native environment such as, for example, a host cell. In embodiments, “isolated” refers to a nucleic acid, polynucleotide, polypeptide, protein, or other component that is partially or completely separated from components with which it is normally associated (other proteins, nucleic acids, cells, etc.).

As used herein, the terms “biomolecule” or “analyte” refer to an agent (e.g., a compound, macromolecule, or small molecule), and the like derived from a biological system (e.g., an organism, a cell, or a tissue). The biomolecule may contain multiple individual components that collectively construct the biomolecule, for example, in embodiments, the biomolecule is a polynucleotide wherein the polynucleotide is composed of nucleotide monomers. The biomolecule may be or may include DNA, RNA, organelles, carbohydrates, lipids, proteins, or any combination thereof. These components may be extracellular. In some examples, the biomolecule may be referred to as a clump or aggregate of combinations of components. In some instances, the biomolecule may include one or more constituents of a cell but may not include other constituents of the cell. In embodiments, a biomolecule is a molecule produced by a biological system (e.g., an organism). The biomolecule may be any substance (e.g. molecule) or entity that is desired to be detected by the method of the invention. The biomolecule is the “target” of the assay method of the invention. The biomolecule may accordingly be any compound that may be desired to be detected, for example a peptide or protein, or nucleic acid molecule or a small molecule, including organic and inorganic molecules. The biomolecule may be a cell or a microorganism, including a virus, or a fragment or product thereof. Biomolecules of particular interest may thus include proteinaceous molecules such as peptides, polypeptides, proteins or prions or any molecule which includes a protein or polypeptide component, etc., or fragments thereof. The biomolecule may be a single molecule or a complex that contains two or more molecular subunits, which may or may not be covalently bound to one another, and which may be the same or different. Thus, in addition to cells or microorganisms, such a complex biomolecule may also be a protein complex. Such a complex may thus be a homo- or hetero-multimer. Aggregates of molecules e.g., proteins may also be target analytes, for example aggregates of the same protein or different proteins. The biomolecule may also be a complex between proteins or peptides and nucleic acid molecules such as DNA or RNA. Of particular interest may be the interactions between proteins and nucleic acids, e.g., regulatory factors, such as transcription factors, and interactions between DNA or RNA molecules.

As used herein, “biomaterial” refers to any biological material produced by an organism. In some embodiments, biomaterial includes secretions, extracellular matrix, proteins, lipids, organelles, membranes, cells, portions thereof, and combinations thereof. In some embodiments, cellular material includes secretions, extracellular matrix, proteins, lipids, organelles, membranes, cells, portions thereof, and combinations thereof. In some embodiments, biomaterial includes viruses. In some embodiments, the biomaterial is a replicating virus and thus includes virus infected cells. In embodiments, a biological sample includes biomaterials.

As used herein, the term “primed template DNA molecule” refers to a template DNA molecule which is associated with a primer (a short polynucleotide) that can serve as a starting point for DNA synthesis.

As used herein, the term “incorporating a nucleotide into a nucleic acid sequence” refers to the process of joining a cognate nucleotide to a nucleic acid primer by formation of a phosphodiester bond. In embodiments, methods of incorporating a nucleotide into a nucleic acid sequence includes combining in a reaction vessel: (i) a nucleic acid template, (ii) a nucleotide solution comprising a plurality of nucleotides, and (iii) a polymerase.

As used herein, the term “primer-template hybridization complex” refers to a double stranded nucleic acid complex formed as a result of a hybridization event between a DNA template molecule and a primer. In embodiments, the formation of a template complex enables elongation at the 3′ end of the primer.

A nucleic acid can be amplified by a suitable method. The term “amplified” as used herein refers to subjecting a target nucleic acid in a sample to a process that linearly or exponentially generates amplicon nucleic acids having the same or substantially the same (e.g., substantially identical) nucleotide sequence as the target nucleic acid, or segment thereof, and/or a complement thereof. In embodiments an amplified product (e.g., an amplicon) can contain one or more additional and/or different nucleotides than the template sequence, or portion thereof, from which the amplicon was generated (e.g., a primer can contain “extra” nucleotides (such as a 5′ portion that does not hybridize to the template), or one or more mismatched bases within a hybridizing portion of the primer). Amplification according to the present disclosure encompasses any means by which at least a part of at least one target nucleic acid is reproduced, typically in a template-dependent manner, including without limitation, a broad range of techniques for amplifying nucleic acid sequences, either linearly or exponentially. Illustrative means for performing an amplifying step include ligase chain reaction (LCR), ligase detection reaction (LDR), ligation followed by Q-replicase amplification, PCR, primer extension, strand displacement amplification (SDA), hyperbranched strand displacement amplification, multiple displacement amplification (MDA), nucleic acid strand-based amplification (NASBA), two-step multiplexed amplifications, rolling circle amplification (RCA), and the like, including multiplex versions and combinations thereof, for example but not limited to, OLA (oligonucleotide ligation assay)/PCR, PCR/OLA, LDR/PCR, PCR/PCR/LDR, PCR/LDR, LCR/PCR, PCR/LCR (also known as combined chain reaction—CCR), and the like.

In some embodiments, amplification includes at least one cycle of the sequential procedures of: annealing at least one primer with complementary or substantially complementary sequences in at least one target nucleic acid; synthesizing at least one strand of nucleotides in a template-dependent manner using a polymerase; and optionally denaturing the newly-formed nucleic acid duplex to separate the strands. The cycle may or may not be repeated. Amplification can include thermocycling or can be performed isothermally.

As used herein, the term “rolling circle amplification (RCA)” refers to a nucleic acid amplification reaction that amplifies a circular nucleic acid template (e.g., single-stranded DNA circles) via a rolling circle process. Rolling circle amplification reaction is initiated by the hybridization of a primer to a circular, often single-stranded, nucleic acid template. The nucleic acid polymerase then extends the primer that is hybridized to the circular nucleic acid template by continuously progressing around the circular nucleic acid template to replicate the sequence of the nucleic acid template over and over again (rolling circle mechanism). The rolling circle amplification typically produces concatemers including tandem repeat units of the circular nucleic acid template sequence. The rolling circle amplification may be a linear RCA (LRCA), exhibiting linear amplification kinetics (e.g., RCA using a single specific primer), or may be an exponential RCA (ERCA) exhibiting exponential amplification kinetics. Rolling circle amplification may also be performed using multiple primers (multiply primed rolling circle amplification or MPRCA) leading to hyper-branched concatemers. For example, in a double-primed RCA, one primer may be complementary, as in the linear RCA, to the circular nucleic acid template, whereas the other may be complementary to the tandem repeat unit nucleic acid sequences of the RCA product. Consequently, the double-primed RCA may proceed as a chain reaction with exponential (geometric) amplification kinetics featuring a ramifying cascade of multiple-hybridization, primer-extension, and strand-displacement events involving both the primers. This often generates a discrete set of concatemeric, double-stranded nucleic acid amplification products. The rolling circle amplification may be performed in vitro under isothermal conditions using a suitable nucleic acid polymerase.

A nucleic acid can be amplified by a thermocycling method or by an isothermal amplification method. In some embodiments a rolling circle amplification method is used. In some embodiments amplification takes place on a solid support (e.g., within a flow cell) where a nucleic acid, nucleic acid library or portion thereof is immobilized. In certain sequencing methods, a nucleic acid library is added to a flow cell and immobilized by hybridization to anchors under suitable conditions. This type of nucleic acid amplification is often referred to as solid phase amplification. In some embodiments of solid phase amplification, all or a portion of the amplified products are synthesized by an extension initiating from an immobilized primer. Solid phase amplification reactions are analogous to standard solution phase amplifications except that at least one of the amplification oligonucleotides (e.g., primers) is immobilized on a solid support.

In some embodiments solid phase amplification includes a nucleic acid amplification reaction including only one species of oligonucleotide primer immobilized to a surface or substrate. In certain embodiments solid phase amplification includes a plurality of different immobilized oligonucleotide primer species. In some embodiments solid phase amplification may include a nucleic acid amplification reaction including one species of oligonucleotide primer immobilized on a solid surface and a second different oligonucleotide primer species in solution. Multiple different species of immobilized or solution-based primers can be used.

As used herein, the terms “cluster” and “colony” are used interchangeably to refer to a discrete site on a solid support that includes a plurality of immobilized polynucleotides and a plurality of immobilized complementary polynucleotides. The term “clustered array” refers to an array formed from such clusters or colonies. In this context the term “array” is not to be understood as requiring an ordered arrangement of clusters. The term “array” is used in accordance with its ordinary meaning in the art, and refers to a population of different molecules that are attached to one or more solid-phase substrates such that the different molecules can be differentiated from each other according to their relative location. An array can include different molecules that are each located at different addressable features on a solid-phase substrate. The molecules of the array can be nucleic acid primers, nucleic acid probes, nucleic acid templates or nucleic acid enzymes such as polymerases or ligases. Arrays useful in the invention can have densities that ranges from about 2 different features to many millions, billions or higher. The density of an array can be from 2 to as many as a billion or more different features per square cm. For example an array can have at least about 100 features/cm², at least about 1,000 features/cm², at least about 10,000 features/cm², at least about 100,000 features/cm², at least about 10,000,000 features/cm², at least about 100,000,000 features/cm², at least about 1,000,000,000 features/cm², at least about 2,000,000,000 features/cm² or higher. In embodiments, the arrays have features at any of a variety of densities including, for example, at least about 10 features/cm², 100 features/cm², 500 features/cm², 1,000 features/cm², 5,000 features/cm², 10,000 features/cm², 50,000 features/cm², 100,000 features/cm², 1,000,000 features/cm², 5,000,000 features/cm², or higher.

Provided herein are methods and compositions for analyzing a sample (e.g., sequencing nucleic acids within a sample). A sample (e.g., a sample including nucleic acid) can be obtained from a suitable subject. A sample can be isolated or obtained directly from a subject or part thereof. In some embodiments, a sample is obtained indirectly from an individual or medical professional. A sample can be any specimen that is isolated or obtained from a subject or part thereof. A sample can be any specimen that is isolated or obtained from multiple subjects. Non-limiting examples of specimens include fluid or tissue from a subject, including, without limitation, blood or a blood product (e.g., serum, plasma, platelets, buffy coats, or the like), umbilical cord blood, chorionic villi, amniotic fluid, cerebrospinal fluid, spinal fluid, lavage fluid (e.g., lung, gastric, peritoneal, ductal, ear, arthroscopic), a biopsy sample, celocentesis sample, cells (blood cells, lymphocytes, placental cells, stem cells, bone marrow derived cells, embryo or fetal cells) or parts thereof (e.g., mitochondrial, nucleus, extracts, or the like), urine, feces, sputum, saliva, nasal mucous, prostate fluid, lavage, semen, lymphatic fluid, bile, tears, sweat, breast milk, breast fluid, the like or combinations thereof. A fluid or tissue sample from which nucleic acid is extracted may be acellular (e.g., cell-free). Non-limiting examples of tissues include organ tissues (e.g., liver, kidney, lung, thymus, adrenals, skin, bladder, reproductive organs, intestine, colon, spleen, brain, the like or parts thereof), epithelial tissue, hair, hair follicles, ducts, canals, bone, eye, nose, mouth, throat, ear, nails, the like, parts thereof or combinations thereof. A sample may include cells or tissues that are normal, healthy, diseased (e.g., infected), and/or cancerous (e.g., cancer cells). A sample obtained from a subject may include cells or cellular material (e.g., nucleic acids) of multiple organisms (e.g., virus nucleic acid, fetal nucleic acid, bacterial nucleic acid, parasite nucleic acid).

In some embodiments, a sample includes one or more nucleic acids, or fragments thereof. A sample can include nucleic acids obtained from one or more subjects. In some embodiments, the nucleic acid is in a cell or tissue. In some embodiments, the nucleic acid is obtained from a cell or tissue. In some embodiments a sample includes nucleic acid obtained from a single subject. In some embodiments, a sample includes a mixture of nucleic acids. A mixture of nucleic acids can include two or more nucleic acid species having different nucleotide sequences, different fragment lengths, different origins (e.g., genomic origins, cell or tissue origins, subject origins, the like or combinations thereof), or combinations thereof.

A subject can be any living or non-living organism, including but not limited to a human, non-human animal, plant, bacterium, fungus, virus or protist. A subject may be any age (e.g., an embryo, a fetus, infant, child, adult). A subject can be of any sex (e.g., male, female, or combination thereof). A subject may be pregnant. In some embodiments, a subject is a mammal. In some embodiments, a subject is a human subject. A subject can be a patient (e.g., a human patient). In some embodiments a subject is suspected of having a genetic variation or a disease or condition associated with a genetic variation.

The methods and kits of the present disclosure may be applied, mutatis mutandis, to the sequencing of RNA, or to determining the identity of a ribonucleotide.

As used herein, the term “ligase” is used in accordance with its plain ordinary meaning and refers to an enzyme capable of catalyzing the joining of a 5′-phosphate end to the 3′-hydroxyl end of one or more polynucleotide strand(s) (e.g., under appropriate reaction conditions). In embodiments, the ligase refers to an ATP-dependent ligase. The ligation reaction by an ATP-dependent ligase includes (1) the formation of a covalent bond between a lysine residue in the active site of the ligase and AMP, (2) the transfer of the AMP to the 5′-phosphate terminated end of a nicked DNA to generate a DNA-adenylate intermediate, and (3) the formation of a covalent bond between the 3′-hydroxyl terminated end and the nicked DNA and release of AMP (see, e.g., Sriskana et al. Nucleic Acids Res. 1998 Jan. 15; 26(2):525-31; Ho et al. J Virol. 1997 March; 71(3):1931-7; Samai et al. J Biol Chem. 2012 Aug. 17; 287(34):28609-18; Odell et al. Mol Cell. 2000 November; 6(5):1183-93, each of which are incorporated herein by reference in their entirety). Examples of DNA ligases include, but are not limited to, DNA ligase I, DNA ligase IIIa, DNA ligase IV, poxvirus ligases, and PBCV-1 DNA ligase.

“Ligase variants” is used in accordance with its plain ordinary meaning and refers to non-naturally occurring (e.g., synthetic or recombinant) ligase enzymes. For example, ligase variants may be constructed by synthetic methods and/or mutated parent enzymes such as truncated ligases, DNA ligases attached to a protein or peptide domain, ligases with one or more point mutations in the amino acid sequence. In embodiments, the ligase variant is a variant of a DNA ligase. In embodiments, the ligase variant is a variant of an RNA ligase. Variants of the wild type or parent ligase have been engineered by mutating residues using site-directed or random mutagenesis methods known in the art. The variant is expressed in an expression system such as E. coli by methods known in the art.

As used herein, the term “polynucleotide-binding polypeptide” refers to an independently folded protein domain that includes a structural motif that is capable of recognizing and binding to a double-stranded or single-stranded polynucleotide. A polynucleotide-binding polypeptide is capable of recognizing specific polynucleotide sequences, which enables the polynucleotide-binding polypeptide to bind to the double-stranded polynucleotide or single-stranded polynucleotide with high affinity and specificity. Structural examples of a polynucleotide-binding polypeptide includes but is not limited to, helix-turn-helix domain, zinc finger domain, leucine zipper domain, and helix-loop-helix domain. Described herein are methods and compositions directed to a recombinant ligase covalently attached to a polynucleotide-binding polypeptide. Exemplary examples of polynucleotide-binding polypeptide include, but are not limited to, Ss07d, hLig3 zinc finger, Sac7d, and Sac7e (see, e.g., Kalichuk et al. Sci Rep. 2016 Nov. 17:6:37274 and Bauer et al. PLoS One. 2017 Dec. 28; 12(12):e0190062, each of which are incorporated herein by reference in their entirety).

The terms “bioconjugate group,” “bioconjugate reactive moiety,” and “bioconjugate reactive group” refer to a chemical moiety which participates in a reaction to form a bioconjugate linker (e.g., covalent linker). Non-limiting examples of bioconjugate reactive groups and the resulting bioconjugate reactive linkers may be found in the Bioconjugate Table below:

| Bioconjugate reactive group 1 | Bioconjugate reactive group 2 | |
|---|---|---|
| (e.g., electrophilic | (e.g., nucleophilic bioconjugate | Resulting Bioconjugate |
| bioconjugate reactive moiety) | reactive moiety) | reactive linker |
| activated esters | amines/anilines | carboxamides |
| acrylamides | thiols | thioethers |
| acyl azides | amines/anilines | carboxamides |
| acyl halides | amines/anilines | carboxamides |
| acyl halides | alcohols/phenols | esters |
| acyl nitriles | alcohols/phenols | esters |
| acyl nitriles | amines/anilines | carboxamides |
| aldehydes | amines/anilines | imines |
| aldehydes or ketones | hydrazines | hydrazones |
| aldehydes or ketones | hydroxylamines | oximes |
| alkyl halides | amines/anilines | alkyl amines |
| alkyl halides | carboxylic acids | esters |
| alkyl halides | thiols | thioethers |
| alkyl halides | alcohols/phenols | ethers |
| alkyl sulfonates | thiols | thioethers |
| alkyl sulfonates | carboxylic acids | esters |
| alkyl sulfonates | alcohols/phenols | ethers |
| anhydrides | alcohols/phenols | esters |
| anhydrides | amines/anilines | carboxamides |
| aryl halides | thiols | thiophenols |
| aryl halides | amines | aryl amines |
| aziridines | thiols | thioethers |
| boronates | glycols | boronate esters |
| carbodiimides | carboxylic acids | N-acylureas or anhydrides |
| diazoalkanes | carboxylic acids | esters |
| epoxides | thiols | thioethers |
| haloacetamides | thiols | thioethers |
| haloplatinate | amino | platinum complex |
| haloplatinate | heterocycle | platinum complex |
| haloplatinate | thiol | platinum complex |
| halotriazines | amines/anilines | aminotriazines |
| halotriazines | alcohols/phenols | triazinyl ethers |
| halotriazines | thiols | triazinyl thioethers |
| imido esters | amines/anilines | amidines |
| isocyanates | amines/anilines | ureas |
| isocyanates | alcohols/phenols | urethanes |
| isothiocyanates | amines/anilines | thioureas |
| maleimides | thiols | thioethers |
| phosphoramidites | alcohols | phosphite esters |
| silyl halides | alcohols | silyl ethers |
| sulfonate esters | amines/anilines | alkyl amines |
| sulfonate esters | thiols | thioethers |
| sulfonate esters | carboxylic acids | esters |
| sulfonate esters | alcohols | ethers |
| sulfonyl halides | amines/anilines | sulfonamides |
| sulfonyl halides | phenols/alcohols | sulfonate esters |

As used herein, the term “bioconjugate reactive moiety” and “bioconjugate reactive group” refers to a moiety or group capable of forming a bioconjugate (e.g., covalent linker) as a result of the association between atoms or molecules of bioconjugate reactive groups. The association can be direct or indirect. For example, a conjugate between a first bioconjugate reactive group (e.g., —NH₂, —COOH, —N-hydroxysuccinimide, or -maleimide) and a second bioconjugate reactive group (e.g., sulfhydryl, sulfur-containing amino acid, amine, amine sidechain containing amino acid, or carboxylate) provided herein can be direct, e.g., by covalent bond or linker (e.g., a first linker of second linker), or indirect, e.g., by non-covalent bond (e.g., electrostatic interactions (e.g., ionic bond, hydrogen bond, halogen bond), van der Waals interactions (e.g., dipole-dipole, dipole-induced dipole, London dispersion), ring stacking (pi effects), hydrophobic interactions and the like). In embodiments, bioconjugates or bioconjugate linkers are formed using bioconjugate chemistry (i.e., the association of two bioconjugate reactive groups) including, but are not limited to nucleophilic substitutions (e.g., reactions of amines and alcohols with acyl halides, active esters), electrophilic substitutions (e.g., enamine reactions) and additions to carbon-carbon and carbon-heteroatom multiple bonds (e.g., Michael reaction, Diels-Alder addition). These and other useful reactions are discussed in, for example, March, ADVANCED ORGANIC CHEMISTRY, 3rd Ed., John Wiley & Sons, New York, 1985; Hermanson, BIOCONJUGATE TECHNIQUES, Academic Press, San Diego, 1996; and Feeney et al., MODIFICATION OF PROTEINS; Advances in Chemistry Series, Vol. 198, American Chemical Society, Washington, D.C., 1982. In embodiments, the first bioconjugate reactive group (e.g., maleimide moiety) is covalently attached to the second bioconjugate reactive group (e.g., a sulfhydryl). In embodiments, the first bioconjugate reactive group (e.g., haloacetyl moiety) is covalently attached to the second bioconjugate reactive group (e.g., a sulfhydryl). In embodiments, the first bioconjugate reactive group (e.g., pyridyl moiety) is covalently attached to the second bioconjugate reactive group (e.g., a sulfhydryl). In embodiments, the first bioconjugate reactive group (e.g., —N-hydroxysuccinimide moiety) is covalently attached to the second bioconjugate reactive group (e.g., an amine). In embodiments, the first bioconjugate reactive group (e.g., maleimide moiety) is covalently attached to the second bioconjugate reactive group (e.g., a sulfhydryl). In embodiments, the first bioconjugate reactive group (e.g., -sulfo-N-hydroxysuccinimide moiety) is covalently attached to the second bioconjugate reactive group (e.g., an amine).

Useful bioconjugate reactive groups used for bioconjugate chemistries herein include, for example: (a) carboxyl groups and various derivatives thereof including, but not limited to, N-hydroxysuccinimide esters, N-hydroxybenztriazole esters, acid halides, acyl imidazoles, thioesters, p-nitrophenyl esters, alkyl, alkenyl, alkynyl and aromatic esters; (b) hydroxyl groups which can be converted to esters, ethers, aldehydes, etc.; (c) haloalkyl groups wherein the halide can be later displaced with a nucleophilic group such as, for example, an amine, a carboxylate anion, thiol anion, carbanion, or an alkoxide ion, thereby resulting in the covalent attachment of a new group at the site of the halogen atom; (d) dienophile groups which are capable of participating in Diels-Alder reactions such as, for example, maleimido or maleimide groups; (e) aldehyde or ketone groups such that subsequent derivatization is possible via formation of carbonyl derivatives such as, for example, imines, hydrazones, semicarbazones or oximes, or via such mechanisms as Grignard addition or alkyllithium addition; (f) sulfonyl halide groups for subsequent reaction with amines, for example, to form sulfonamides; (g) thiol groups, which can be converted to disulfides, reacted with acyl halides, or bonded to metals such as gold, or react with maleimides; (h) amine or sulfhydryl groups (e.g., present in cysteine), which can be, for example, acylated, alkylated or oxidized; (i) alkenes, which can undergo, for example, cycloadditions, acylation, Michael addition, etc.; (j) epoxides, which can react with, for example, amines and hydroxyl compounds; (k) phosphoramidites and other standard functional groups useful in nucleic acid synthesis; (l) metal silicon oxide bonding; (m) metal bonding to reactive phosphorus groups (e.g., phosphines) to form, for example, phosphate diester bonds; (n) azides coupled to alkynes using copper catalyzed cycloaddition click chemistry; (o) biotin conjugate can react with avidin or strepavidin to form a avidin-biotin complex or streptavidin-biotin complex.

“Histidine-tag” or “His-tag” refers to a polypeptide sequence comprising between two (His₂) and ten (His₁₀) consecutive histidine residues. In embodiments, the His-tag facilitates affinity purification of recombinant proteins by enabling specific binding to metal ions, such as nickel (Ni²⁺) or cobalt (Co²⁺), immobilized on chromatographic resins (e.g., immobilized metal affinity chromatography, IMAC). In embodiments, the His-tag may be positioned at the N-terminus, the C-terminus, or within an internal region of a target protein, depending on the design of the expression construct. In embodiments, the His-tag facilitates purification, detection, or immobilization of the tagged protein while minimally affecting its biological function.

### II. Compositions & Kits

Provided herein are compositions including ligases and variants thereof exhibiting increased thermostability, increased activity at higher temperatures (e.g., up to 45° C.), efficient at ligation in situ, increased stability during purification (result in in greater yield), and reduced propensity for aggregation relative to a control (e.g., wild type ligase or parent ligase). Mutations in the ligase described herein variously include one or more changes to amino acid residues present in the polypeptide sequence. Additions, substitutions, or deletions are all examples of mutations that are used to generate mutant polypeptides. Substitutions in some instances include the exchange of one amino acid for an alternative amino acid, and such alternative amino acids differ from the original amino acid with regard to size, shape, conformation, or chemical structure. Mutations in some instances are conservative or non-conservative. Conservative mutations comprise the substitution of an amino acid with an amino acid that possesses similar chemical properties. Additions often comprise the insertion of one or more amino acids at the N-terminal, C-terminal, or internal positions of the polypeptide. In some embodiments, additions include fusion polypeptides, wherein one or more additional polypeptides (i.e., a polypeptide from a different source) is connected (e.g., covalently linked to the N- or C-terminus) to the ligase as described herein. Such additional polypeptides include domains with additional activity, or sequences with additional function (e.g., improve expression, aid purification, improve solubility, attach to a solid support, or other function).

Wild type ligase sequences are typical initial sequences for protein or enzyme engineering to generate ligase variants. In some embodiments, a polypeptide differs from a wild-type sequence (naturally occurring) by at least one amino acid. Any number of mutations is introduced into a polypeptide or portion of a polypeptide described herein, such as 1, 2, 3, 4, 5, 6, 7, 8, 9, 10, 20, 50, or more than 50 mutations. In embodiments, the ligase differs from a wild-type sequence by at least two amino acids. In embodiments, the ligase differs from a wild-type sequence by at least three, four, five, or at least six amino acids.

Certain nicks in double-stranded nucleic acid molecules (e.g., DNA-DNA or RNA-DNA molecules) may be resealed by a ligase. Ligase enzymes form a phosphodiester bond between a 5′ phosphoryl moiety and a directly adjacent 3′ hydroxyl, using either ATP or NAD⁺ as an energy source. Resealing a nicked strand is illustrated in Scheme 1.

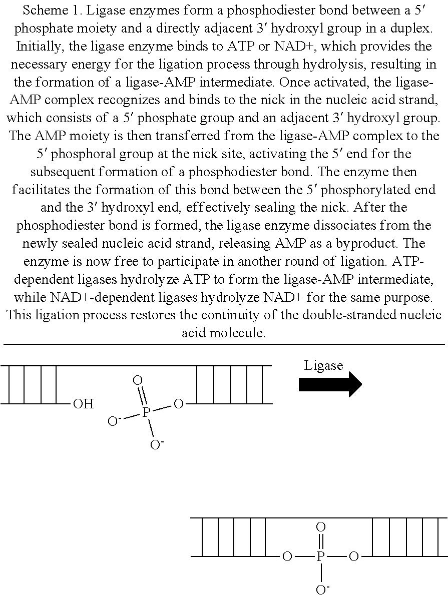

Ligases establish a new chemical bond in a duplex polynucleotide. DNA ligases are a particular type of ligase that join DNA fragments together. The ability to catalyze the ligation of adjacent single stranded DNA splinted by a complementary RNA strand is a property that is specific to certain DNA ligases, e.g., PBCV-1.

In an aspect is provided a ligase including one or more amino acid substitutions as described herein. In embodiments, the ligase includes an amino acid sequence that is at least 80%, 85%, 90%, 95%, or 99% identical to SEQ ID NO:1 and includes an amino acid substitution, wherein the amino acid substitution is: serine, threonine, methionine, alanine, valine, tyrosine, tryptophan, or arginine at an amino acid position corresponding to position 22 of SEQ ID NO:1.

In an aspect is provided a ligase including an amino acid sequence that is at least 80%, 85%, 90%, 95%, or 99% identical to SEQ ID NO:1; including a serine, threonine, methionine, alanine, valine, tyrosine, tryptophan, or arginine at an amino acid position corresponding to position 22 of SEQ ID NO:1. In embodiments, the ligase includes a serine at the amino acid position corresponding to position 22 of SEQ ID NO:1. In embodiments, the ligase includes a threonine at the amino acid position corresponding to position 22 of SEQ ID NO:1. In embodiments, the ligase includes a methionine at the amino acid position corresponding to position 22 of SEQ ID NO:1. In embodiments, the ligase includes an alanine at the amino acid position corresponding to position 22 of SEQ ID NO:1. In embodiments, the ligase includes a valine at the amino acid position corresponding to position 22 of SEQ ID NO:1. In embodiments, the ligase includes a tyrosine at the amino acid position corresponding to position 22 of SEQ ID NO:1. In embodiments, the ligase includes a tryptophan at the amino acid position corresponding to position 22 of SEQ ID NO:1. In embodiments, the ligase includes a tyrosine at the amino acid position corresponding to position 22 of SEQ ID NO:1. In embodiments, the ligase includes an arginine at the amino acid position corresponding to position 22 of SEQ ID NO:1.

In an aspect is provided a ligase including an amino acid sequence that is at least 80%, 85%, 90%, 95%, or 99% identical to SEQ ID NO:1. In embodiments, the ligase includes an amino acid sequence that is at least 80% identical to a continuous 250 amino acid sequence within SEQ ID NO: 1. In embodiments, the ligase includes an amino acid sequence that is at least 80% identical to a continuous 200 amino acid sequence within SEQ ID NO:1. In embodiments, the ligase includes an amino acid sequence that is at least 80% identical to a continuous 100 amino acid sequence within SEQ ID NO:1. In embodiments, the ligase includes an amino acid sequence that is at least 90% identical to a continuous 250 amino acid sequence within SEQ ID NO:1. In embodiments, the ligase includes an amino acid sequence that is at least 95% identical to a continuous 250 amino acid sequence within SEQ ID NO:1. In an aspect is provided a ligase including an amino acid sequence that is at least 80%, 85%, 90%, 95%, or 99% identical to SEQ ID NO:17. In embodiments, the ligase includes an amino acid sequence that is at least 80% identical to a continuous 250 amino acid sequence within SEQ ID NO:17.

In an aspect is provided a ligase including an amino acid sequence that is at least 80%, 85%, 90%, 95%, or 99% identical to SEQ ID NO:1; including a serine, threonine, methionine, alanine, or valine at an amino acid position corresponding to position 22 of SEQ ID NO:1.

In an aspect is provided a ligase including an amino acid sequence that is at least 80%, 85%, 90%, 95%, or 99% identical to SEQ ID NO:1; covalently attached to a hydrophilic polymer, such as polyethylene glycol, polypropylene glycol, or polyoxazoline. In embodiments, the ligase is covalently attached to a polyethylene glycol moiety. In embodiments the polyethylene glycol moiety includes

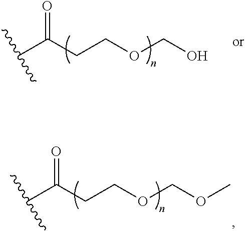

wherein n is 2 to 24. In embodiments, n is 1, 2, 3, 4, 5, 6, 7, 8, 9, 10, 11, 12, 13, 14, 15, 16, 17, 18, 19, 20 21, 22, 23, or 24. In embodiments, n is 6, 12, 18, or 24. In embodiments, n is 6. In embodiments, n is 12. In embodiments, n is 18. In embodiments, n is 24. In embodiments, the ligase includes a plurality of polyethylene glycol moieties. In embodiments, the polyethylene glycol moiety is covalently attached to the ligase via a bioconjugate linker. For example, a PEG-NHS (polyethylene glycol-N-hydroxysuccinimide) ester may be used to link the ligase to PEG, wherein the NHS ester group enables a stable amide bond with a primary amine present on the ligase. In embodiments, the polyethylene glycol moiety is covalently linked to the N-terminus or C-terminus. In embodiments, the polyethylene glycol moiety is covalently linked to an internal residue of the ligase. The stoichiometry of the conjugation reaction to form PEG-ligase conjugates may include one equivalent of ligase (e.g., a ligase containing one or more bioconjugate-reactive moieties, such as primary amines on lysine residues) and at least 0.5 equivalents of a functionalized polyethylene glycol (PEG) moiety. The PEG moiety may be modified to include a reactive group, such as an N-hydroxysuccinimide (NHS) ester, maleimide, or aldehyde, capable of forming a covalent linkage with complementary reactive sites on the ligase. Additional examples may include at least 1.0 equivalent, at least 1.5 equivalents, at least 2.0 equivalents, at least 2.5 equivalents, at least 3.0 equivalents, at least 3.5 equivalents, at least 4.0 equivalents, at least 4.5 equivalents, at least 5.0 equivalents, or at least 5.5 equivalents of PEG relative to ligase. In embodiments, the stoichiometry of the conjugation reaction may include one equivalent of ligase and between about 0.5 and about 5.5 equivalents of PEG, for example, between about 1.5 and about 2.5 equivalents, between about 2.0 and about 3.0 equivalents, between about 2.5 and about 3.5 equivalents, between about 3.0 and about 4.0 equivalents, between about 3.5 and about 4.5 equivalents, between about 4.0 and about 5.0 equivalents, or between about 4.5 and about 5.5 equivalents of PEG. In embodiments, the stoichiometry of the conjugation reaction may be adjusted to achieve PEG-ligase conjugates that retain sufficient enzymatic activity and substrate binding affinity. A suitable PEG reagent may include functional groups that react with lysine F-amines, N-terminal α-amines, cysteines, or engineered residues. For example, the PEG reagent may be prepared using a bifunctional linker containing a first reactive component that covalently modifies the PEG and a second reactive component that forms a stable linkage with a complementary functional group on the ligase, such as a thiol, amine, hydrazine, or oxyamine.

In embodiments, the ligase includes one or more substitutions at amino acid position corresponding to position 7, 8, 10, 23, 24, 25, 27, 29, 30, 32, 42, 65, 67, 75, 92, 93, 94, 95, 96, 97, 98, 99, 100, 107, 108, 109, 110, 111, 112, 113, 114, 115, 116, 117, 118, 119, 140, 161, 164, 165, 166, 167, 171, 172, 173, 174, 175, 176, 186, 188, 190, 228, 234, 276, 285, 286, 293, and/or 297 of SEQ ID NO:1 as described herein. In embodiments, the ligase includes a substitution at amino acid position corresponding to position 140, 243, 244, and/or 246. In embodiments, the ligase includes a substitution at amino acid position corresponding to position 140. In embodiments, the ligase includes a substitution at amino acid position corresponding to position 243. In embodiments, the ligase includes a substitution at amino acid position corresponding to position 244. In embodiments, the ligase includes a substitution at amino acid position corresponding to position 246. In embodiments, the ligase includes substitutions at amino acid positions corresponding to positions 140 and 243. In embodiments, the ligase includes substitutions at amino acid positions corresponding to positions 140 and 244. In embodiments, the ligase includes substitutions at amino acid positions corresponding to positions 140 and 246. In embodiments, the ligase includes substitutions at amino acid positions corresponding to positions 243 and 244. In embodiments, the ligase includes substitutions at amino acid positions corresponding to positions 243 and 246. In embodiments, the ligase includes substitutions at amino acid positions corresponding to positions 244 and 246. In embodiments, the ligase includes substitutions at amino acid positions corresponding to positions 140, 243, and 244. In embodiments, the ligase includes substitutions at amino acid positions corresponding to positions 140, 243, and 246. In embodiments, the ligase includes substitutions at amino acid positions corresponding to positions 140, 244, and 246. In embodiments, the ligase includes substitutions at amino acid positions corresponding to positions 243, 244, and 246. In embodiments, the ligase includes substitutions at amino acid positions corresponding to positions 140, 243, 244, and 246. In embodiments, the ligase includes the amino acid substitution mutations V140K, V243K, V244K, and S246E relative to SEQ ID NO:1.

In embodiments, the ligase includes tryptophan, phenylalanine, or histidine at the amino acid position corresponding to position 7 of SEQ ID NO:1. In embodiments, the ligase includes tryptophan at the amino acid position corresponding to position 7 of SEQ ID NO:1. In embodiments, the ligase includes phenylalanine at the amino acid position corresponding to position 7 of SEQ ID NO:1. In embodiments, the ligase includes histidine at the amino acid position corresponding to position 7 of SEQ ID NO:1.

In embodiments, the ligase includes tryptophan, phenylalanine, tyrosine, glutamic acid, or aspartic acid at the amino acid position corresponding to position 164 of SEQ ID NO:1. In embodiments, the ligase includes tryptophan at the amino acid position corresponding to position 164 of SEQ ID NO:1. In embodiments, the ligase includes phenylalanine at the amino acid position corresponding to position 164 of SEQ ID NO:1. In embodiments, the ligase includes tyrosine at the amino acid position corresponding to position 164 of SEQ ID NO:1. In embodiments, the ligase includes glutamic acid at the amino acid position corresponding to position 164 of SEQ ID NO:1. In embodiments, the ligase includes aspartic acid at the amino acid position corresponding to position 164 of SEQ ID NO:1.

An amino acid sequence alignment can provide amino acid positions corresponding to position 22 of SEQ ID NO:1. For example, the amino acid position 22 of SEQ ID NO:2 corresponds to position 22 of SEQ ID NO:1. In embodiments, the ligase includes a serine at the amino acid position corresponding to position 22 of SEQ ID NO:1. In embodiments, the ligase includes a threonine at the amino acid position corresponding to position 22 of SEQ ID NO:1. In embodiments, the ligase includes a methionine at the amino acid position corresponding to position 22 of SEQ ID NO:1. In embodiments, the ligase includes a alanine at the amino acid position corresponding to position 22 of SEQ ID NO:1. In embodiments, the ligase includes a valine at the amino acid position corresponding to position 22 of SEQ ID NO:1.

In embodiments, ligase further includes a serine, threonine, methionine, alanine, or valine at the amino acid position corresponding to position 69 of SEQ ID NO:1. An amino acid sequence alignment can provide amino acid positions corresponding to position 69 of SEQ ID NO:1. For example, the amino acid position 69 of SEQ ID NO:2 corresponds to position 69 of SEQ ID NO:1. In embodiments, the ligase includes a serine at the amino acid position corresponding to position 69 of SEQ ID NO:1. In embodiments, the ligase includes a threonine at the amino acid position corresponding to position 69 of SEQ ID NO:1. In embodiments, the ligase includes a methionine at the amino acid position corresponding to position 69 of SEQ ID NO:1. In embodiments, the ligase includes an alanine at the amino acid position corresponding to position 69 of SEQ ID NO:1. In embodiments, the ligase includes a valine at the amino acid position corresponding to position 69 of SEQ ID NO:1.

In embodiments, ligase further includes a serine, threonine, methionine, alanine, or valine at the amino acid position corresponding to position 298 of SEQ ID NO:1. An amino acid sequence alignment can provide amino acid positions corresponding to position 298 of SEQ ID NO:1. For example, the amino acid position 298 of SEQ ID NO:2 corresponds to position 298 of SEQ ID NO: 1. In embodiments, the ligase further includes a serine at the amino acid position corresponding to position 298 of SEQ ID NO:1. In embodiments, the ligase further includes a threonine at the amino acid position corresponding to position 298 of SEQ ID NO:1. In embodiments, the ligase further includes a methionine at the amino acid position corresponding to position 298 of SEQ ID NO:1. In embodiments, the ligase further includes an alanine at the amino acid position corresponding to position 298 of SEQ ID NO:1. In embodiments, the ligase further includes a valine at the amino acid position corresponding to position 298 of SEQ ID NO:1.

In embodiments, the ligase includes a serine, threonine, methionine, alanine, or valine at the amino acid position corresponding to position 22 of SEQ ID NO:1; a serine, threonine, methionine, alanine, or valine at the amino acid position corresponding to position 69 of SEQ ID NO:1; and a serine, threonine, methionine, alanine, or valine at the amino acid position corresponding to position 298 of SEQ ID NO:1. In embodiments, the ligase includes a serine or threonine at the amino acid position corresponding to position 22 of SEQ ID NO:1; a serine or threonine at the amino acid position corresponding to position 69 of SEQ ID NO: 1; and a serine or threonine at the amino acid position corresponding to position 298 of SEQ ID NO:1. In embodiments, the ligase includes a serine at the amino acid position corresponding to position 22 of SEQ ID NO:1; a serine at the amino acid position corresponding to position 69 of SEQ ID NO:1; and a serine at the amino acid position corresponding to position 298 of SEQ ID NO:1.

In an aspect is provided a ligase including an amino acid sequence that is at least 80%, 85%, 90%, 95%, or 99% identical to SEQ ID NO:1; comprising a serine, threonine, methionine, alanine, valine, tyrosine, tryptophan, or arginine at an amino acid position corresponding to position 69 of SEQ ID NO:1.

In an aspect is provided a ligase including an amino acid sequence that is at least 80%, 85%, 90%, 95%, or 99% identical to SEQ ID NO:1; comprising a serine, threonine, methionine, alanine, valine, tyrosine, tryptophan, or arginine at an amino acid position corresponding to position 298 of SEQ ID NO:1.

In an aspect is provided a ligase including an amino acid sequence that is at least 80%, 85%, 90%, 95%, or 99% identical to a continuous 100, 200, or 250 amino acid sequence within SEQ ID NO:1. In embodiments, the ligase includes an amino acid sequence that is at least 80%, 85%, 90%, 95%, or 99% identical to a continuous 100 amino acid sequence within SEQ ID NO:1. In embodiments, the ligase includes an amino acid sequence that is at least 80%, 85%, 90%, 95%, or 99% identical to a continuous 200 amino acid sequence within SEQ ID NO:1. In embodiments, the ligase includes an amino acid sequence that is at least 80%, 85%, 90%, 95%, or 99% identical to a continuous 250 amino acid sequence within SEQ ID NO:1.

In the context of this application, one having ordinary skill in the art would understand that SEQ ID NO:1 and SEQ ID NO:2 are related as SEQ ID NO:1 provides the amino acid sequence of wild type PBCV NYs1 ligase (referred herein as PB-1), and SEQ ID NO:2 provides the amino acid sequence of wild type PBCV NYs1 ligase including a C-terminus truncation at amino acid position 298 of SEQ ID NO:1. One having ordinary skill in the art would understand that SEQ ID NO:1 and SEQ ID NO:19 are related as SEQ ID NO:1 provides the amino acid sequence of wild type PBCV NYs1 ligase (referred herein as PB-1), and SEQ ID NO:19 provides the amino acid sequence of wild type PBCV NYs1 ligase of SEQ ID NO:1 including a His-tag and linker at the N-terminus. Any amino acid after amino acid position 1 in SEQ ID NO:1 is shifted by 10 amino acid positions to identify the corresponding amino acid position in SEQ ID NO:19. For example, amino acid position 244 in SEQ ID NO:1 corresponds to amino acid position 254 in SEQ ID NO:19. In embodiments, the ligase includes a V244K mutation, V243K mutation, S246E mutation, and a V140K mutation.

In embodiments, the ligase including an amino acid sequence that is at least 80%, 85%, 90%, 95%, or 99% identical to SEQ ID NO:1 includes one or more of the following amino acid substitutions: C22A, C22M, C22S, C22T, C22V, C69A, C69M, C69S, C69T, C69V, C298A, C298M, C298S, C298T, C298V, D297A, D297E, D297F, D2971, D297M, D297N, D297V, D297W, D297Y, D29A, D29E, D29F, D29I, D29M, D29N, D29V, D29W, D29Y, D65A, D65F, D65I, D65M, D65V, D65W, D65Y, E161A, E161F, E1611, E161M, E161V, E161W, E161Y, E67A, E67F, E671, E67M, E67V, E67W, E67Y, F98A, F98D, F98E, F98H, F98K, F98N, F98Q, F98V, F98R, G30A, G30P, K186A, K186F, K186H, K186N, K186Q, K186S, K186T, K186W, K186R, K186Y, K188A, K188F, K188H, K188N, K188Q, K188S, K188T, K188R, K188W, K188Y, K27A, K27F, K27H, K27N, K27Q, K27R, K27S, K27T, K27W, K27Y, M164A, M164D, M164E, M164F, M164G, M164H, M164K, M164P, M164N, M164Q, M164R, M164S, M164V, M164W, M164Y, R176A, R176D, R176E, R176F, R176H, R176K, R176S, R176T, R176M, R176N, R176Q, R176T, R176V, R176W, R293A, R293D, R293E, R293F, R293H, R293K, R293M, R293N, R293Q, R293S, R293T, R293T, R293V, R293W, R32A, R32D, R32E, R32F, R32H, R32K, R32M, R32N, R32Q, R32S, R32T, R32V, R32W, R42A, R42D, R42E, R42F, R42H, R42K, R42M, R42N, R42Q, R42S, R42T, R42V, R42W, T25A, T25F, T25H, T25K, T25N, T25Q, T25R, T25V, T25W, and/or T25Y.

In embodiments, the ligase includes one or more substitutions at amino acid position corresponding to position 22, 25, 27, 29, 30, 32, 42, 65, 67, 69, 98, 140, 161, 164, 176, 186, 188, 293, 297, and/or 298 of SEQ ID NO:1. In embodiments, the ligase described herein includes one or more substitutions at amino acid position corresponding to position 22, 25, 27, 29, 30, 32, 42, 65, 67, 69, 98, 161, 164, 176, 186, 188, 293, 297, and/or 298 of SEQ ID NO:2.

In embodiments, the ligase includes one or more substitutions at amino acid position corresponding to position 7, 25, 27, 29, 30, 32, 42, 65, 67, 98, 161, 164, 172, 176, 186, 188, 228, 234, 285, 293, and/or 297 of SEQ ID NO:1. In embodiments, the ligase described herein includes one or more substitutions at amino acid position corresponding to position 7, 25, 27, 29, 30, 32, 42, 65, 67, 98, 161, 164, 172, 176, 186, 188, 228, 234, 285, 293, and/or 297 of SEQ ID NO:2.

In embodiments, the ligase includes one or more substitutions at amino acid position corresponding to position 8, 10, 23, 24, 75, 92, 93, 94, 95, 96, 97, 99, 100, 107, 108, 109, 110, 111, 112, 113, 114, 115, 116, 117, 118, 119, 165, 166, 167, 171, 173, 174, 175, 190, 276, and/or 286 of SEQ ID NO: 1. In embodiments, the ligase described herein further includes one or more substitutions at amino acid position corresponding to position 8, 10, 23, 24, 75, 92, 93, 94, 95, 96, 97, 99, 100, 107, 108, 109, 110, 111, 112, 113, 114, 115, 116, 117, 118, 119, 165, 166, 167, 171, 173, 174, 175, 190, 276, and/or 286 of SEQ ID NO:2.

In embodiments, the ligase includes one or more substitutions at amino acid position corresponding to position 7, 8, 10, 23, 24, 25, 27, 29, 30, 32, 42, 65, 67, 75, 92, 93, 94, 95, 96, 97, 98, 99, 100, 107, 108, 109, 110, 111, 112, 113, 114, 115, 116, 117, 118, 119, 161, 164, 165, 166, 167, 171, 172, 173, 174, 175, 176, 186, 188, 190, 228, 234, 276, 285, 286, 293, and/or 297 of SEQ ID NO:1. In embodiments, the ligase described herein further includes one or more substitutions at amino acid position corresponding to position 7, 8, 10, 23, 24, 25, 27, 29, 30, 32, 42, 65, 67, 75, 92, 93, 94, 95, 96, 97, 98, 99, 100, 107, 108, 109, 110, 111, 112, 113, 114, 115, 116, 117, 118, 119, 161, 164, 165, 166, 167, 171, 172, 173, 174, 175, 176, 186, 188, 190,228, 234, 276, 285, 286, 293, and/or 297 of SEQ ID NO:2.

In embodiments, the ligase includes one or more substitutions at amino acid position corresponding to position 15, 32, 42, 43, 48, 53, 54, 85, 86, 87, 88, 118, 121, 150, 153, 171, 176, 181, 190, 201, 224, 227, 237, 241, 242, 245, 247, 254, 256, 257, 258, 259, 265, 270, 271, 283, 285, 294, 295, 296, and/or 297 of SEQ ID NO:1. In embodiments, the ligase includes one or more substitutions at amino acid position corresponding to position 15, 32, 42, 43, 48, 53, 54, 85, 86, 87, 88, 118, 121, 150, 153, 171, 176, 181, 190, 201, 224, 227, 237, 241, 242, 245, 247, 254, 256, 257, 258, 259, 265, 270, 271, 283, 285, 294, 295, 296, and/or 297 of SEQ ID NO:2.

In embodiments, the ligase includes a tryptophan, phenylalanine, or histidine at the amino acid position corresponding to position 7 of SEQ ID NO:1. In embodiments, the ligase described herein further includes a tryptophan, phenylalanine, or histidine at the amino acid position corresponding to position 7 of SEQ ID NO:2.

In embodiments, the ligase includes a tryptophan, phenylalanine, tyrosine, glutamic acid, or aspartic acid at the amino acid position corresponding to position 164 of SEQ ID NO: 1. In embodiments, the ligase described herein further includes a tryptophan, phenylalanine, tyrosine, glutamic acid, or aspartic acid at the amino acid position corresponding to position 164 of SEQ ID NO:2.

In embodiments, the ligase includes one or more substitutions at amino acid positions at least one of the following pairs of amino acids: Arg153 and Thr201; Arg176 and Asp297; Arg285 and Glu237; Arg32 and Glu296; Arg42 and Asp297; Arg48 and Asp258; Arg48 and Ser265; Asn118 and Met88; Asn224 and Phe54; Asn224 and Thr53; Asp258 and Gly85; Cys283 and Cys283; Glu181 and Gly242; Glu271 and Lys190; Glu295 and Thr43; His86 and Asp254; Ile257 and Ser256; Lys15 and Tyr227; Lys190 and Trp270; Ser245 and Glu247; Ser256 and Ser245; Thr121 and Lys87; Thr201 and Gln150; Thr259 and Pro294; and/or Thr43 and Glu295 of SEQ ID NO:1. In embodiments, the ligase described herein further includes one or more substitutions at amino acid positions at least one of the following pairs of amino acids: Arg153 and Thr201; Arg176 and Asp297; Arg285 and Glu237; Arg32 and Glu296; Arg42 and Asp297; Arg48 and Asp258; Arg48 and Ser265; Asn118 and Met88; Asn224 and Phe54; Asn224 and Thr53; Asp258 and Gly85; Cys283 and Cys283; Glu181 and Gly242; Glu271 and Lys190; Glu295 and Thr43; His86 and Asp254; Ile257 and Ser256; Lys15 and Tyr227; Lys190 and Trp270; Ser245 and Glu247; Ser256 and Ser245; Thr121 and Lys87; Thr201 and Gln150; Thr259 and Pro294; and/or Thr43 and Glu295 of SEQ ID NO:2. In embodiments, the ligase includes one or more substitutions at amino acid positions Arg153 and Thr201 of SEQ ID NO:1. In embodiments, the ligase includes one or more substitutions at amino acid positions Arg176 and Asp297 of SEQ ID NO:1. In embodiments, the ligase includes one or more substitutions at amino acid positions Arg285 and Glu237 of SEQ ID NO:1. In embodiments, the ligase includes one or more substitutions at amino acid positions Arg32 and Glu296 of SEQ ID NO:1. In embodiments, the ligase includes one or more substitutions at amino acid positions Arg42 and Asp297 of SEQ ID NO:1. In embodiments, the ligase includes one or more substitutions at amino acid positions Arg48 and Asp258 of SEQ ID NO:1. In embodiments, the ligase includes one or more substitutions at amino acid positions Arg48 and Ser265 of SEQ ID NO:1. In embodiments, the ligase includes one or more substitutions at amino acid positions Asn118 and Met88 of SEQ ID NO:1. In embodiments, the ligase includes one or more substitutions at amino acid positions Asn224 and Phe54 of SEQ ID NO:1. In embodiments, the ligase includes one or more substitutions at amino acid positions Asn224 and Thr53 of SEQ ID NO:1. In embodiments, the ligase includes one or more substitutions at amino acid positions Asp258 and Gly85 of SEQ ID NO:1. In embodiments, the ligase includes one or more substitutions at amino acid positions Cys283 and Cys283 of SEQ ID NO:1. In embodiments, the ligase includes one or more substitutions at amino acid positions Glu181 and Gly242 of SEQ ID NO:1. In embodiments, the ligase includes one or more substitutions at amino acid positions Glu271 and Lys190 of SEQ ID NO:1. In embodiments, the ligase includes one or more substitutions at amino acid positions Glu295 and Thr43 of SEQ ID NO:1. In embodiments, the ligase includes one or more substitutions at amino acid positions His86 and Asp254 of SEQ ID NO:1. In embodiments, the ligase includes one or more substitutions at amino acid positions Ile257 and Ser256 of SEQ ID NO:1. In embodiments, the ligase includes one or more substitutions at amino acid positions Lys15 and Tyr227 of SEQ ID NO:1. In embodiments, the ligase includes one or more substitutions at amino acid positions Lys190 and Trp270 of SEQ ID NO:1. In embodiments, the ligase includes one or more substitutions at amino acid positions Ser245 and Glu247 of SEQ ID NO:1. In embodiments, the ligase includes one or more substitutions at amino acid positions Ser256 and Ser245 of SEQ ID NO:1. In embodiments, the ligase includes one or more substitutions at amino acid positions Thr121 and Lys87 of SEQ ID NO:1. In embodiments, the ligase includes one or more substitutions at amino acid positions Thr201 and Gln150 of SEQ ID NO:1. In embodiments, the ligase includes one or more substitutions at amino acid positions Thr259 and Pro294 of SEQ ID NO:1. In embodiments, the ligase includes one or more substitutions at amino acid positions Thr43 and Glu295 of SEQ ID NO: 1.

In embodiments, the ligase includes one or more substitutions at amino acid positions at least one of the following pairs of amino acids: Arg176 and Asp297; Arg285 and Glu237; Arg42 and Asp297; Arg48 and Asp258; His86 and Asp254; Lys171 and Asp241 of SEQ ID NO:1. In embodiments, the ligase described herein further includes one or more substitutions at amino acid positions at least one of the following pairs of amino acids: Arg176 and Asp297; Arg285 and Glu237; Arg42 and Asp297; Arg48 and Asp258; His86 and Asp254; Lys171 and Asp241 of SEQ ID NO:2.

In embodiments, the ligase includes an aspartic acid or glutamic acid at amino acid position corresponding to position 94 of SEQ ID NO:1. In embodiments, the ligase includes an aspartic acid at amino acid position corresponding to position 94 of SEQ ID NO:1. In embodiments, the ligase includes a glutamic acid at amino acid position corresponding to position 94 of SEQ ID NO:1.

In embodiments, the ligase includes a methionine, aspartic acid, or glutamic acid at amino acid position corresponding to position 138 of SEQ ID NO:1. In embodiments, the ligase includes a methionine acid at amino acid position corresponding to position 138 of SEQ ID NO:1. In embodiments, the ligase includes an aspartic acid at amino acid position corresponding to position 138 of SEQ ID NO:1. In embodiments, the ligase includes a glutamic acid at amino acid position corresponding to position 138 of SEQ ID NO:1.

In embodiments, the ligase includes an arginine or lysine at amino acid position corresponding to position 140 of SEQ ID NO:1. In embodiments, the ligase includes an arginine at amino acid position corresponding to position 140 of SEQ ID NO:1. In embodiments, the ligase includes a lysine at amino acid position corresponding to position 140 of SEQ ID NO:1. In embodiments, the ligase includes a glycine, isoleucine, leucine, or alanine at the amino acid position corresponding to position 140 of SEQ ID NO:1.

In embodiments, the ligase includes an arginine or lysine at amino acid position corresponding to position 198 of SEQ ID NO:1. In embodiments, the ligase includes an arginine at amino acid position corresponding to position 198 of SEQ ID NO:1. In embodiments, the ligase includes a lysine at amino acid position corresponding to position 198 of SEQ ID NO:1.

In embodiments, the ligase includes an aspartic acid or glutamic acid at amino acid position corresponding to position 215 of SEQ ID NO:1. In embodiments, the ligase includes an aspartic acid at amino acid position corresponding to position 215 of SEQ ID NO:1. In embodiments, the ligase includes a glutamic acid at amino acid position corresponding to position 215 of SEQ ID NO:1.

In embodiments, the ligase includes a lysine, arginine, methionine, threonine, aspartic acid, or glutamic acid at amino acid position corresponding to position 243 of SEQ ID NO:1. In embodiments, the ligase includes a lysine at amino acid position corresponding to position 243 of SEQ ID NO:1. In embodiments, the ligase includes an arginine at amino acid position corresponding to position 243 of SEQ ID NO:1. In embodiments, the ligase includes a methionine at amino acid position corresponding to position 243 of SEQ ID NO:1. In embodiments, the ligase includes a threonine at amino acid position corresponding to position 243 of SEQ ID NO:1. In embodiments, the ligase includes an aspartic acid at amino acid position corresponding to position 243 of SEQ ID NO:1. In embodiments, the ligase includes a glutamic acid at amino acid position corresponding to position 243 of SEQ ID NO:1. In embodiments, the ligase includes a glycine, isoleucine, leucine, or alanine at the amino acid position corresponding to position 243 of SEQ ID NO:1.

In embodiments, the ligase includes a lysine, arginine, methionine, threonine, aspartic acid, or glutamic acid at amino acid position corresponding to position 244 of SEQ ID NO:1. In embodiments, the ligase includes a lysine at amino acid position corresponding to position 244 of SEQ ID NO:1. In embodiments, the ligase includes an arginine at amino acid position corresponding to position 244 of SEQ ID NO:1. In embodiments, the ligase includes a methionine at amino acid position corresponding to position 244 of SEQ ID NO:1. In embodiments, the ligase includes a threonine at amino acid position corresponding to position 244 of SEQ ID NO:1. In embodiments, the ligase includes an aspartic acid at amino acid position corresponding to position 244 of SEQ ID NO:1. In embodiments, the ligase includes a glutamic acid at amino acid position corresponding to position 244 of SEQ ID NO:1. In embodiments, the ligase includes a glycine, isoleucine, leucine, or alanine at the amino acid position corresponding to position 244 of SEQ ID NO:1.

In embodiments, the ligase includes a lysine or arginine at amino acid position corresponding to position 245 of SEQ ID NO:1. In embodiments, the ligase includes a lysine at amino acid position corresponding to position 245 of SEQ ID NO:1. In embodiments, the ligase includes an arginine at amino acid position corresponding to position 245 of SEQ ID NO:1.

In embodiments, the ligase includes a lysine, arginine, proline, aspartic acid, or glutamic acid at amino acid position corresponding to position 246 of SEQ ID NO:1. In embodiments, the ligase includes a lysine at amino acid position corresponding to position 246 of SEQ ID NO:1. In embodiments, the ligase includes an arginine at amino acid position corresponding to position 246 of SEQ ID NO:1. In embodiments, the ligase includes a proline at amino acid position corresponding to position 246 of SEQ ID NO:1. In embodiments, the ligase includes an aspartic acid at amino acid position corresponding to position 246 of SEQ ID NO:1. In embodiments, the ligase includes a glutamic acid at amino acid position corresponding to position 246 of SEQ ID NO:1. In embodiments, the ligase includes a glycine, isoleucine, leucine, or alanine at the amino acid position corresponding to position 246 of SEQ ID NO:1.

In embodiments, the ligase includes a proline, aspartic acid, or glutamic acid at amino acid position corresponding to position 248 of SEQ ID NO:1. In embodiments, the ligase includes a proline at amino acid position corresponding to position 248 of SEQ ID NO:1. In embodiments, the ligase includes an aspartic acid at amino acid position corresponding to position 248 of SEQ ID NO:1. In embodiments, the ligase includes a glutamic acid at amino acid position corresponding to position 248 of SEQ ID NO:1.

In embodiments, the ligase includes a lysine, arginine, methionine, threonine, aspartic acid, and glutamic acid at amino acid position corresponding to position 286 of SEQ ID NO:1. In embodiments, the ligase includes a lysine at amino acid position corresponding to position 286 of SEQ ID NO:1. In embodiments, the ligase includes an arginine at amino acid position corresponding to position 286 of SEQ ID NO:1. In embodiments, the ligase includes a methionine at amino acid position corresponding to position 286 of SEQ ID NO:1. In embodiments, the ligase includes a threonine at amino acid position corresponding to position 286 of SEQ ID NO:1. In embodiments, the ligase includes an aspartic acid at amino acid position corresponding to position 286 of SEQ ID NO:1. In embodiments, the ligase includes a glutamic acid at amino acid position corresponding to position 286 of SEQ ID NO:1.

In embodiments, the ligase includes a lysine, arginine, aspartic acid, and glutamic acid at amino acid position corresponding to position 290 of SEQ ID NO:1. In embodiments, the ligase includes a lysine at amino acid position corresponding to position 290 of SEQ ID NO:1. In embodiments, the ligase includes an arginine at amino acid position corresponding to position 290 of SEQ ID NO:1. In embodiments, the ligase includes an aspartic acid at amino acid position corresponding to position 290 of SEQ ID NO:1. In embodiments, the ligase includes a glutamic acid at amino acid position corresponding to position 290 of SEQ ID NO:1.

In embodiments, the ligase includes the following amino acid substitution mutations relative to SEQ ID NO:1: V244K and S246E; V244K and V140K; V244K and V243K; V244R and S246E; V244R and V140K; V244R and V243K; S246E and V140K; S246E and V243K; or V140K and V243K. In embodiments, the ligase includes the amino acid substitution mutations V244K and S246E relative to SEQ ID NO:1. In embodiments, the ligase includes the amino acid substitution mutations V244K and V140K relative to SEQ ID NO:1. In embodiments, the ligase includes the amino acid substitution mutations V244K and V243K relative to SEQ ID NO:1. In embodiments, the ligase includes the amino acid substitution mutations V244R and S246E relative to SEQ ID NO:1. In embodiments, the ligase includes the amino acid substitution mutations V244R and V140K relative to SEQ ID NO:1. In embodiments, the ligase includes the amino acid substitution mutations V244R and V243K relative to SEQ ID NO:1. In embodiments, the ligase includes the amino acid substitution mutations S246E and V140K relative to SEQ ID NO:1. In embodiments, the ligase includes the amino acid substitution mutations S246E and V243K relative to SEQ ID NO:1. In embodiments, the ligase includes the amino acid substitution mutations V140K and V243K relative to SEQ ID NO:1.

In an aspect is provided a ligase including an amino acid sequence that is at least 80%, 85%, 90%, 95%, or 99% identical to SEQ ID NO:2; including a serine, threonine, methionine, alanine, valine, tyrosine, tryptophan, or arginine at an amino acid position corresponding to position 22 of SEQ ID NO:2. In embodiments, the ligase includes a serine at the amino acid position corresponding to position 22 of SEQ ID NO:2. In embodiments, the ligase includes a threonine at the amino acid position corresponding to position 22 of SEQ ID NO:2. In embodiments, the ligase includes a methionine at the amino acid position corresponding to position 22 of SEQ ID NO:2. In embodiments, the ligase includes a alanine at the amino acid position corresponding to position 22 of SEQ ID NO:2. In embodiments, the ligase includes a valine at the amino acid position corresponding to position 22 of SEQ ID NO:2.

In embodiments, ligase includes a serine, threonine, methionine, alanine, or valine at the amino acid position corresponding to position 69 of SEQ ID NO:2. An amino acid sequence alignment can provide amino acid positions corresponding to position 69 of SEQ ID NO:2. For example, the amino acid position 69 of SEQ ID NO:1 corresponds to position 69 of SEQ ID NO:2. In embodiments, the ligase includes a serine at the amino acid position corresponding to position 69 of SEQ ID NO:2. In embodiments, the ligase includes a threonine at the amino acid position corresponding to position 69 of SEQ ID NO:2. In embodiments, the ligase includes a methionine at the amino acid position corresponding to position 69 of SEQ ID NO:2. In embodiments, the ligase includes an alanine at the amino acid position corresponding to position 69 of SEQ ID NO:2. In embodiments, the ligase includes a valine at the amino acid position corresponding to position 69 of SEQ ID NO:2.

In embodiments, the ligase includes a serine, threonine, methionine, alanine, or valine at the amino acid position corresponding to position 22 of SEQ ID NO:2 and a serine, threonine, methionine, alanine, or valine at the amino acid position corresponding to position 69 of SEQ ID NO:2. In embodiments, the ligase includes a serine or threonine at the amino acid position corresponding to position 22 of SEQ ID NO:2 and a serine or threonine at the amino acid position corresponding to position 69 of SEQ ID NO:2. In embodiments, the ligase includes a serine at the amino acid position corresponding to position 22 of SEQ ID NO:2 and a serine at the amino acid position corresponding to position 69 of SEQ ID NO:2.

In embodiments, a 6×His6-tag is attached to the C-terminus of the ligase as described herein. In embodiments, a 6×His-tag is attached to the N-terminus of the ligase as described herein. It is understood that the presence of a 6×His tag enables the isolation of peptide or protein products directly from ligation reaction mixtures by Ni-NTA affinity column purification. For example, common polyhistidine tags are formed of six histidine (6×His tag) residues which are added at the N-terminus preceded by methionine or C-terminus before a stop codon. Alternative polycationic sequences include alternating histidine and glutamine (e.g., three sets of HQ, referred to as an HQ tag) or alternating histidine and asparagine (e.g., six sets of HN, referred to as an HN tag). In embodiments, the 6×His6-tag is attached to a linker including glycine and serine.

In general, purification tags may be added to the ligase (recombinantly or chemically) and include, e.g., polycationic tags (polyhistidine tags or His6-tags), GST tags (Glutathione-S-transferase), MBP (maltose binding protein) tags, biotin, avidin, GST sequences, BTag sequences, S tags, SNAP-tags, enterokinase sites, thrombin sites, antibodies or antibody domains, antibody fragments, antigens, receptors, receptor domains, and/or receptor fragments.

In an aspect is provided a ligase including an amino acid sequence that is at least 80%, 85%, 90%, 95%, or 99% identical to SEQ ID NO:1 attached (e.g., fused via a covalent attachment) to a polynucleotide-binding polypeptide. In embodiments, the ligase is recombinant.

In an aspect is provided a ligase including an amino acid sequence that is at least 80%, 85%, 90%, 95%, or 99% identical to SEQ ID NO:2 attached (e.g., fused via a covalent attachment) to a polynucleotide-binding polypeptide. In embodiments, the ligase is recombinant.

In embodiments, the ligase is covalently attached to the polynucleotide-binding polypeptide. In embodiments, the ligase is covalently attached to a Sso7d polypeptide. In embodiments, the ligase is covalently attached to a Sac7d polypeptide. In embodiments, the ligase is covalently attached to a Sac7e polypeptide. In embodiments, the ligase is covalently attached to a Mse7 polypeptide. In embodiments, the ligase is covalently attached to a Mcu7 polypeptide. In embodiments, the ligase is covalently attached to a Aho7a polypeptide. In embodiments, the ligase is covalently attached to a Aho7b polypeptide. In embodiments, the ligase is covalently attached to a Aho7c polypeptide. In embodiments, the ligase is covalently attached to a Sto7 polypeptide. In embodiments, the ligase is covalently attached to a Ssh7b polypeptide. In embodiments, the ligase is covalently attached to a Sis7a polypeptide. In embodiments, the ligase is covalently attached to a Sis7b polypeptide. In embodiments, the ligase is covalently attached to a Ssh7a polypeptide. In embodiments, the ligase is covalently attached to a nucleoid-associated protein HU-alpha polypeptide. In embodiments, the ligase is covalently attached to a Sso7d polypeptide at the N-terminal of the ligase (e.g., SEQ ID NO:1). In embodiments, the ligase includes a linker between the ligase and the polynucleotide-binding polypeptide. In embodiments, the ligase is covalently attached to a Sso7d polypeptide at the C-terminal of the ligase (e.g., SEQ ID NO:1). For example, in embodiments, the linker includes the amino acid sequence: GTGGGG (SEQ ID NO:21). In embodiments, the ligase is covalently attached to the polynucleotide-binding polypeptide. In embodiments, the ligase covalently attached to the polynucleotide-binding polypeptide includes a sequence that is 80%, 85%, 90%, 95%, or 99% identical to SEQ ID NO:22. In embodiments, the ligase covalently attached to the polynucleotide-binding polypeptide includes a sequence that is 80%, 85%, 90%, 95%, or 99% identical to SEQ ID NO:23.

One having ordinary skill in the art would understand that SEQ ID NO:2 and SEQ ID NO:20 are related as SEQ ID NO:2 provides the amino acid sequence of wild type PBCV NYs1 ligase including a C-terminus truncation at amino acid position 298 of SEQ ID NO:1, and SEQ ID NO:20 provides the amino acid sequence of the PBCV NYs1 ligase of SEQ ID NO:2 including a His-tag and linker at the N-terminus. One having ordinary skill in the art would understand that SEQ ID NO:22 and SEQ ID NO:1 are related as SEQ ID NO:1 provides the amino acid sequence of wild type PBCV NYs1 ligase (referred herein as PB-1), and SEQ ID NO:22 provides the amino acid sequence of wild type PBCV NYs1 ligase covalently attached to a His-tag, flexible linker, amino acid sequence of a Sso7d polypeptide, and linker at the N-terminus of the ligase (referred herein as PB-9). Any amino acid position after amino acid position 1 in SEQ ID NO:1 is shifted by 79 amino acid position to identify the corresponding amino acid position in SEQ ID NO:22. For example, amino acid position 244 in SEQ ID NO:1 corresponds to amino acid position 321 in SEQ ID NO:22. Similarly, one having ordinary skill in the art would understand that SEQ ID NO:22 and SEQ ID NO:19 are related as SEQ ID NO:19 provides the amino acid sequence of wild type PBCV NYs1 ligase covalently attached to a His-tag and a linker at the N-terminus (referred herein as PB-1_HT), and SEQ ID NO:22 provides the amino acid sequence of wild type PBCV NYs1 ligase covalently attached to a His-tag, flexible linker, amino acid sequence of a Sso7d polypeptide, and linker at the N-terminus of the ligase (referred herein as PB-9). Any amino acid position after amino acid position 1 in SEQ ID NO:19 is shifted by 69 amino acid position to identify the corresponding amino acid position in SEQ ID NO:22. For example, amino acid position 254 in SEQ ID NO:19 corresponds to amino acid position 323 in SEQ ID NO:22.

In embodiments, the polynucleotide-binding polypeptide includes a sequence that is at least 80%, 85%, 90%, 95%, or 99% identical to a continuous 20 nucleotide sequence within SEQ ID NO:3. In embodiments, the polynucleotide-binding polypeptide includes a sequence that is at least 80%, 85%, 90%, 95%, or 99% identical to a continuous 30 nucleotide sequence within SEQ ID NO:3. In embodiments, the polynucleotide-binding polypeptide includes a sequence that is at least 80%, 85%, 90%, 95%, or 99% identical to a continuous 40 nucleotide sequence within SEQ ID NO:3. In embodiments, the polynucleotide-binding polypeptide includes a sequence that is at least 80%, 85%, 90%, 95%, or 99% identical to a continuous 50 nucleotide sequence within SEQ ID NO:3. In embodiments, the polynucleotide-binding polypeptide includes a sequence that is at least 80%, 85%, 90%, 95%, or 99% identical to a continuous 20 nucleotide sequence within SEQ ID NO:3. In embodiments, the polynucleotide-binding polypeptide includes a sequence that is at least 80%, 85%, 90%, 95%, or 99% identical to a continuous 60 nucleotide sequence within SEQ ID NO:3.

In embodiments, the polynucleotide-binding polypeptide includes a sequence that is at least 80%, 85%, 90%, 95%, or 99% identical to at least a continuous 30 nucleotide sequence within SEQ ID NO:4. In embodiments, the polynucleotide-binding polypeptide includes a sequence that is at least 80%, 85%, 90%, 95%, or 99% identical to at least a continuous 30 nucleotide sequence within SEQ ID NO:5. In embodiments, the polynucleotide-binding polypeptide includes a sequence that is at least 80%, 85%, 90%, 95%, or 99% identical to at least a continuous 30 nucleotide sequence within SEQ ID NO:6. In embodiments, the polynucleotide-binding polypeptide includes a sequence that is at least 80%, 85%, 90%, 95%, or 99% identical to at least a continuous 30 nucleotide sequence within SEQ ID NO:7. In embodiments, the polynucleotide-binding polypeptide includes a sequence that is at least 80%, 85%, 90%, 95%, or 99% identical to at least a continuous 30 nucleotide sequence within SEQ ID NO:8. In embodiments, the polynucleotide-binding polypeptide includes a sequence that is at least 80%, 85%, 90%, 95%, or 99% identical to at least a continuous 30 nucleotide sequence within SEQ ID NO:9. In embodiments, the polynucleotide-binding polypeptide includes a sequence that is at least 80%, 85%, 90%, 95%, or 99% identical to at least a continuous 30 nucleotide sequence within SEQ ID NO: 10. In embodiments, the polynucleotide-binding polypeptide includes a sequence that is at least 80%, 85%, 90%, 95%, or 99% identical to at least a continuous 30 nucleotide sequence within SEQ ID NO:11. In embodiments, the polynucleotide-binding polypeptide includes a sequence that is at least 80%, 85%, 90%, 95%, or 99% identical to at least a continuous 30 nucleotide sequence within SEQ ID NO:12. In embodiments, the polynucleotide-binding polypeptide includes a sequence that is at least 80%, 85%, 90%, 95%, or 99% identical to at least a continuous 30 nucleotide sequence within SEQ ID NO:13. In embodiments, the polynucleotide-binding polypeptide includes a sequence that is at least 80%, 85%, 90%, 95%, or 99% identical to at least a continuous 30 nucleotide sequence within SEQ ID NO:14. In embodiments, the polynucleotide-binding polypeptide includes a sequence that is at least 80%, 85%, 90%, 95%, or 99% identical to at least a continuous 30 nucleotide sequence within SEQ ID NO: 15. In embodiments, the polynucleotide-binding polypeptide includes a sequence that is at least 80%, 85%, 90%, 95%, or 99% identical to at least a continuous 30 nucleotide sequence within SEQ ID NO:25 (e.g., PB-34).

In embodiments, the polynucleotide-binding polypeptide includes a sequence that is at least 85% identical to a continuous 30 nucleotide sequence within SEQ ID NO:4; at least 85% identical to a continuous 30 nucleotide sequence within SEQ ID NO:5; at least 85% identical to a continuous 30 nucleotide sequence within SEQ ID NO:6; at least 85% identical to a continuous 30 nucleotide sequence within SEQ ID NO:7; at least 85% identical to a continuous 30 nucleotide sequence within SEQ ID NO:8; at least 85% identical to a continuous 30 nucleotide sequence within SEQ ID NO:9; at least 85% identical to a continuous 30 nucleotide sequence within SEQ ID NO: 10; at least 85% identical to a continuous 30 nucleotide sequence within SEQ ID NO:11; at least 85% identical to a continuous 30 nucleotide sequence within SEQ ID NO:12; at least 85% identical to a continuous 30 nucleotide sequence within SEQ ID NO:13; at least 85% identical to a continuous 30 nucleotide sequence within SEQ ID NO:14; at least 85% identical to a continuous 30 nucleotide sequence within SEQ ID NO:15; or at least 85% identical to a continuous 30 nucleotide sequence within SEQ ID NO:25.

In embodiments, the polynucleotide-binding polypeptide includes a sequence that is at least 85% identical to a continuous 30 nucleotide sequence within SEQ ID NO:4. In embodiments, the polynucleotide-binding polypeptide includes a sequence that is at least 85% identical to a continuous 30 nucleotide sequence within SEQ ID NO:5. In embodiments, the polynucleotide-binding polypeptide includes a sequence that is at least 85% identical to a continuous 30 nucleotide sequence within SEQ ID NO:6. In embodiments, the polynucleotide-binding polypeptide includes a sequence that is at least 85% identical to a continuous 30 nucleotide sequence within SEQ ID NO:7. In embodiments, the polynucleotide-binding polypeptide includes a sequence that is at least 85% identical to a continuous 30 nucleotide sequence within SEQ ID NO:8. In embodiments, the polynucleotide-binding polypeptide includes a sequence that is at least 85% identical to a continuous 30 nucleotide sequence within SEQ ID NO:9. In embodiments, the polynucleotide-binding polypeptide includes a sequence that is at least 85% identical to a continuous 30 nucleotide sequence within SEQ ID NO:10. In embodiments, the polynucleotide-binding polypeptide includes a sequence that is at least 85% identical to a continuous 30 nucleotide sequence within SEQ ID NO:11. In embodiments, the polynucleotide-binding polypeptide includes a sequence that is at least 85% identical to a continuous 30 nucleotide sequence within SEQ ID NO:12. In embodiments, the polynucleotide-binding polypeptide includes a sequence that is at least 85% identical to a continuous 30 nucleotide sequence within SEQ ID NO:13. In embodiments, the polynucleotide-binding polypeptide includes a sequence that is at least 85% identical to a continuous 30 nucleotide sequence within SEQ ID NO:14. In embodiments, the polynucleotide-binding polypeptide includes a sequence that is or at least 85% identical to a continuous 30 nucleotide sequence within SEQ ID NO: 15. In embodiments, the polynucleotide-binding polypeptide includes a sequence that is at least 85% identical to a continuous 30 nucleotide sequence within SEQ ID NO:4. In embodiments, the polynucleotide-binding polypeptide is SEQ ID NO:5. In embodiments, the polynucleotide-binding polypeptide is SEQ ID NO:6. In embodiments, the polynucleotide-binding polypeptide is SEQ ID NO:7. In embodiments, the polynucleotide-binding polypeptide is SEQ ID NO:8. In embodiments, the polynucleotide-binding polypeptide is SEQ ID NO:9. In embodiments, the polynucleotide-binding polypeptide is SEQ ID NO:10. In embodiments, the polynucleotide-binding polypeptide is SEQ ID NO:11. In embodiments, the polynucleotide-binding polypeptide is SEQ ID NO:12. In embodiments, the polynucleotide-binding polypeptide is SEQ ID NO:13. In embodiments, the polynucleotide-binding polypeptide is SEQ ID NO:14. In embodiments, the polynucleotide-binding polypeptide includes a sequence that is or at least 85% identical to a continuous 30 nucleotide sequence within SEQ ID NO:15. In embodiments, the polynucleotide-binding polypeptide includes a sequence that is or at least 85% identical to a continuous 30 nucleotide sequence within SEQ ID NO:25.

In embodiments, the polynucleotide-binding polypeptide is isolated from Saccharolobus solfataricus. In embodiments, the polynucleotide-binding polypeptide is isolated from Sulfolobus acidocaldarius. In embodiments, the polynucleotide-binding polypeptide is isolated from Metallosphaera sedula. In embodiments, the polynucleotide-binding polypeptide is isolated from Metallosphaera cuprina. In embodiments, the polynucleotide-binding polypeptide is isolated from Acidianus hospitalis. In embodiments, the polynucleotide-binding polypeptide is isolated from Sulfurisphaera tokodaii. In embodiments, the polynucleotide-binding polypeptide is isolated from Sulfolobus islandicus. In embodiments, the polynucleotide-binding polypeptide is isolated from Saccharolobus shibatae.

In embodiments, the polynucleotide-binding polypeptide is a Sso7d polypeptide. In embodiments, the polynucleotide-binding polypeptide is a Sac7d polypeptide. In embodiments, the polynucleotide-binding polypeptide is a Sac7e polypeptide. In embodiments, the polynucleotide-binding polypeptide is a Mse7 polypeptide. In embodiments, the polynucleotide-binding polypeptide is a Mcu7 polypeptide. In embodiments, the polynucleotide-binding polypeptide is a Aho7a polypeptide. In embodiments, the polynucleotide-binding polypeptide is a Aho7b polypeptide. In embodiments, the polynucleotide-binding polypeptide is a Aho7c polypeptide. In embodiments, the polynucleotide-binding polypeptide is a Sto7 polypeptide. In embodiments, the polynucleotide-binding polypeptide is a Ssh7b polypeptide. In embodiments, the polynucleotide-binding polypeptide is a Sis7a polypeptide. In embodiments, the polynucleotide-binding polypeptide is a Sis7b polypeptide. In embodiments, the polynucleotide-binding polypeptide is a Ssh7a polypeptide. In embodiments, the polynucleotide-binding polypeptide is a nucleoid-associated protein HU-alpha polypeptide.

In embodiments, the polypeptide-binding polypeptide is covalently attached (e.g., fused) at the N-terminus of the ligase described herein (e.g., PB-9). In embodiments, the polypeptide-binding polypeptide is covalently attached (e.g., fused) at the C-terminus of the ligase described herein (e.g., PB-8).

In embodiments, the ligase described herein is attached to polymerized units of polyethylene glycol (PEG). In embodiments, the polymerized units of polyethylene glycol (PEG) are attached at the N-terminus of the ligase described herein. In embodiments, the polymerized units of polyethylene glycol (PEG) are attached at the C-terminus of the ligase described herein.

In an aspect is provided a kit. In embodiments, the kit includes a ligase as described herein. In embodiments, the kit includes a wild type ligase or variant thereof. In embodiments, the kit includes the reagents and containers useful for performing the methods as described herein. Generally, the kit includes one or more containers providing a composition and one or more additional reagents (e.g., a buffer suitable for polynucleotide extension). The kit may also include a template nucleic acid (DNA, RNA, and or DNA:RNA hybrid), one or more primer polynucleotides, nucleoside triphosphates (including, for example, deoxyribonucleotides, ribonucleotides, and/or modified nucleotides), buffers, salts, and/or labels (e.g., fluorophores).

In embodiments, the kit includes a solid support (i.e., a substrate), and reagents for sample preparation and purification, amplification, and/or sequencing (e.g., one or more sequencing reaction mixtures). In embodiments, amplification reagents and other reagents may be provided in lyophilized form. In embodiments, amplification reagents and other reagents may be provided in a container which the lyophilized reagent may be reconstituted. In embodiments, sequencing reagents may be provided in lyophilized form. In embodiments, sequencing reagents may be provided in a container which the lyophilized reagent may be reconstituted.

In embodiments, the kit includes a ligase useful for circularizing template polynucleotides. For example, such a kit further includes the following components: (a) reaction buffer for controlling pH and providing an optimized salt composition for the ligase described herein and (b) ligation enzyme cofactors. In embodiments, the kit further includes instructions for use thereof.

In embodiments, kits described herein include a polymerase. In embodiments, the polymerase is a DNA polymerase. In embodiments, the kit includes a strand-displacing polymerase. In embodiments, the polymerase is a DNA polymerase. In embodiments, the DNA polymerase is a thermophilic nucleic acid polymerase. In embodiments, the DNA polymerase is a modified archaeal DNA polymerase.

In embodiments, the kit includes a sequencing solution, hybridization solution, and/or extension solution. In embodiments, the sequencing solution include labeled nucleotides including differently labeled nucleotides, wherein the label (or lack thereof) identifies the type of nucleotide. For example, each adenine nucleotide, or analog thereof; a thymine nucleotide; a cytosine nucleotide, or analog thereof, and a guanine nucleotide, or analog thereof may be labeled with a different fluorescent label.

In embodiments, the kit includes a buffered solution. Typically, the buffered solutions contemplated herein are made from a weak acid and its conjugate base or a weak base and its conjugate acid. For example, sodium acetate and acetic acid are buffer agents that can be used to form an acetate buffer. Other examples of buffer agents that can be used to make buffered solutions include, but are not limited to, Tris, bicine, tricine, HEPES, TES, MOPS, MOPSO and PIPES. Additionally, other buffer agents that can be used in enzyme reactions, hybridization reactions, and detection reactions are known in the art. In embodiments, the buffered solution can include Tris. With respect to the embodiments described herein, the pH of the buffered solution can be modulated to permit any of the described reactions. In some embodiments, the buffered solution can have a pH greater than pH 7.0, greater than pH 7.5, greater than pH 8.0, greater than pH 8.5, greater than pH 9.0, greater than pH 9.5, greater than pH 10, greater than pH 10.5, greater than pH 11.0, or greater than pH 11.5. In other embodiments, the buffered solution can have a pH ranging, for example, from about pH 6 to about pH 9, from about pH 8 to about pH 10, or from about pH 7 to about pH 9. In embodiments, the buffered solution has a pH of about pH 7.0, about pH 7.1, about pH 7.2, about pH 7.3, about pH 7.4, about pH 7.5, about pH 7.6, about pH 7.7, about pH 7.8, about pH 7.9, about pH 8.0, about pH 8.1, about pH 8.2, about pH 8.3, about pH 8.4, or about pH 8.5. In embodiments, the buffered solution can include one or more divalent cations. Examples of divalent cations can include, but are not limited to, Mg²⁺, Mn²⁺, Zn²⁺, and Ca²⁺. In embodiments, the buffered solution can contain one or more divalent cations at a concentration sufficient to permit hybridization of a nucleic acid. In embodiments, the buffered solution can contain one or more divalent cations at a concentration sufficient to permit hybridization of a nucleic acid. In some embodiments, a concentration can be more than about 1 μM, more than about 2 μM, more than about 5 μM, more than about 10 μM, more than about 25 μM, more than about 50 μM, more than about 75 μM, more than about 100 μM, more than about 200 μM, more than about 300 μM, more than about 400 μM, more than about 500 μM, more than about 750 μM, more than about 1 mM, more than about 2 mM, more than about 5 mM, more than about 10 mM, more than about 20 mM, more than about 30 mM, more than about 40 mM, more than about 50 mM, more than about 60 mM, more than about 70 mM, more than about 80 mM, more than about 90 mM, more than about 100 mM, more than about 150 mM, more than about 200 mM, more than about 250 mM, more than about 300 mM, more than about 350 mM, more than about 400 mM, more than about 450 mM, more than about 500 mM, more than about 550 mM, more than about 600 mM, more than about 650 mM, more than about 700 mM, more than about 750 mM, more than about 800 mM, more than about 850 mM, more than about 900 mM, more than about 950 mM or more than about 1 M. In embodiments, the buffered solution includes about 10 mM Tris, about 20 mM Tris, about 30 mM Tris, about 40 mM Tris, or about 50 mM Tris. In embodiments, the buffered solution can include one or more monovalent cations. Examples of monovalent cations can include, but are not limited to, Li⁺, K⁺, and Na⁺. In embodiments the buffered solution includes about 50 mM NaCl, about 75 mM NaCl, about 100 mM NaCl, about 125 mM NaCl, about 150 mM NaCl, about 200 mM NaCl, about 300 mM NaCl, about 400 mM NaCl, or about 500 mM NaCl. In embodiments, the buffered solution includes about 0.05 mM EDTA, about 0.1 mM EDTA, about 0.25 mM EDTA, about 0.5 mM EDTA, about 1.0 mM EDTA, about 1.5 mM EDTA or about 2.0 mM EDTA. In embodiments, the buffered solution includes about 0.01% Triton™ X-100, about 0.025% Triton™ X-100, about 0.05% Triton™ X-100, about 0.1% Triton™ X-100, about 0.25% Triton™ X-100, or about 0.5% Triton™ X-100. In embodiments, the buffered solution includes 0.5% glycerol, 1% glycerol, 2.5% glycerol, 5% glycerol, 10% glycerol, 15 glycerol, 20% glycerol, 10% glycerol, 15% glycerol, 20% glycerol, 25% glycerol, 30% glycerol, 35% glycerol, 40% glycerol, 45% glycerol, or 50% glycerol. In embodiments, the buffered solution includes 0.1 mM DTT, 0.5 mM DTT, 1 mM DTT, 2 mM DTT, 3 mM DTT, 4 mM DTT, 5 mM DTT, 6 mM DTT, 7 mM DTT, 8 mM DTT, 9 mM DTT, 10 mM DTT, 11 mM DTT, 12 mM DTT, 13 mM DTT, 14 mM DTT, 15 mM DTT, 16 mM DTT, 17 mM DTT, 18 mM DTT, 19 mM DTT, or 20 mM DTT. Triton™ is a registered trademark of Dow Chemical Company. In embodiments, the buffered solution includes about 1 mM MgCl₂, about 2 mM MgCl₂, about 3 mM MgCl₂, about 4 mM MgCl₂, about 5 mM MgCl₂, about 6 mM MgCl₂, about 7 mM MgCl₂, about 8 mM MgCl₂, about 9 mM MgCl₂, about 10 mM MgCl₂, about 11 mM MgCl₂, about 12 mM MgCl₂, about 13 mM MgCl₂, about 14 mM MgCl₂, about 15 mM MgCl₂, about 16 mM MgCl₂, about 17 mM MgCl₂, about 18 mM MgCl₂, about 19 mM MgCl₂, or about 20 mM MgCl₂. In embodiments, the buffered solution includes about 0.01 mM ATP, about 0.05 mM ATP, about 0.1 mM ATP, about 0.25 mM ATP, about 0.5 mM ATP, about 0.75 mM ATP, about 1 mM ATP, about 2 mM ATP, about 3 mM ATP, about 4 mM ATP, about 5 mM ATP, about 6 mM ATP, about 7 mM ATP, about 8 mM ATP, about 9 mM ATP, or about 10 mM ATP. In embodiments, the buffered solution includes about 25 mM LiCl, about 50 mM LiCl, about 75 mM LiCl, about 100 mM LiCl, about 125 mM LiCl, about 150 mM LiCl, about 175 mM LiCl, about 200 mM LiCl, about 225 mM LiCl, about 250 mM LiCl, about 275 mM LiCl, about 300 mM LiCl, about 325 mM LiCl, about 350 mM LiCl, about 375 mM LiCl, about 400 mM LiCl, about 425 mM LiCl, about 450 mM LiCl, about 475 mM LiCl, or about 500 mM LiCl.

In embodiments, the buffered solution includes 20 mM Tris pH 8.0, 100 mM NaCl, 0.1 mM EDTA, and 0.025% Triton™ X-100. In embodiments, the buffered solution includes 20 mM Tris pH 8.0, 150 mM NaCl, 0.1 mM EDTA, and 0.025% Triton™ X-100. In embodiments, the buffered solution includes 20 mM Tris pH 8.0, 300 mM NaCl, 0.1 mM EDTA, and 0.025% Triton™ X-100. In embodiments, the buffered solution includes 20 mM Tris pH 8.0, 400 mM NaCl, 0.1 mM EDTA, and 0.025% Triton™ X-100. In embodiments, the buffered solution includes 20 mM Tris pH 8.0, 500 mM NaCl, 0.1 mM EDTA, and 0.025% Triton™ X-100. In embodiments, the buffered solution includes 50 mM Tris pH 8.0, 75 mM LiCl, 3 mM MgCl₂, 0.025% Triton™ X-100, 0.1 mM ATP, and 10 mM DTT. In embodiments, the buffered solution includes 500 mM Tris pH 7.5, 100 mM MgCl₂, 0.25% Triton™ X-100, 10 mM ATP, and 100 mM DTT.

In embodiments, the kit includes, without limitation, nucleic acid primers, probes, adapters, enzymes, and the like, and are each packaged in a container, such as, without limitation, a vial, tube or bottle, in a package suitable for commercial distribution, such as, without limitation, a box, a sealed pouch, a blister pack and a carton. The package typically contains a label or packaging insert indicating the uses of the packaged materials. As used herein, “packaging materials” includes any article used in the packaging for distribution of reagents in a kit, including without limitation containers, vials, tubes, bottles, pouches, blister packaging, labels, tags, instruction sheets and package inserts.

In addition to the above components, the subject kits may further include instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another means would be a computer readable medium, e.g., diskette, CD, digital storage medium, etc., on which the information has been recorded. Yet another means that may be present is a website address which may be used via the Internet to access the information at a removed site. Any convenient means may be present in the kits.

In an aspect is provided a ligase complex, wherein the complex includes a ligase (e.g., a ligase as described herein, for example PB-1) bound to a duplex polynucleotide. In embodiments, the ligase complex is in a cell. In embodiments, the ligase complex is in a tissue. In embodiments, the cell and/or tissue are immobilized to a solid support. In embodiments, the duplex polynucleotide is an RNA-DNA duplex. In embodiments, the duplex polynucleotide is a DNA-DNA duplex. In embodiments, the ligase complex includes a sequence described herein (e.g., SEQ ID NO:1). In embodiments, the ligase is a fusion ligase, wherein the fusion ligase includes the ligase covalently attached (i.e., fused) to a polynucleotide-binding polypeptide (e.g., Sso7d).

### III. Methods

In an aspect is provided a method for detecting polynucleotide sequences including: (a) hybridizing a first polynucleotide sequence to a nucleic acid molecule and hybridizing a second polynucleotide sequence to the nucleic acid molecule (e.g., a padlock probe including DNA); (b) ligating the first polynucleotide sequence and the second polynucleotide sequence together with a ligase to generate a ligated product, wherein the ligase includes an amino acid sequence that is at least 85% identical to SEQ ID NO:1; and (c) detecting the ligated product. In embodiments, the first polynucleotide sequence and the second polynucleotide sequence are part of the same single-stranded polynucleotide sequence, which may be ligated to form a circular polynucleotide. In embodiments, the ligated product includes a barcode sequence.

In embodiments, prior to step (a), the nucleic acid molecule is obtained from a sample. In embodiments, (a)-(c) occur in a cell. In embodiments, (a)-(c) occur in a tissue. In embodiments, steps (a) and (b) occur in a reaction vessel. In embodiments, the reaction vessel is a flow cell. In embodiments, the nucleic acid molecule is in a cell or tissue. In embodiments, the nucleic acid molecule is RNA. In embodiments, the nucleic acid molecule is mRNA. In embodiments, the nucleic acid molecule is rRNA. In embodiments, the nucleic acid molecule is cDNA. In embodiments, the nucleic acid molecule is DNA.

In embodiments, the first polynucleotide sequence and the second polynucleotide sequence form part of a polynucleotide probe, for example, a padlock probe. The polynucleotide probe may be composed of various nucleic acid chemistries, including but not limited to DNA, RNA, or chemically modified nucleic acids such as locked nucleic acid (LNA), 2′-O-methyl RNA, or phosphorothioate-linked oligonucleotides. These modifications may be incorporated to enhance hybridization affinity, nuclease resistance, or structural stability, depending on the specific application. In embodiments, the polynucleotide probe is composed of DNA. In embodiments, the method includes analyzing a target nucleic acid (e.g., an RNA such as an mRNA transcript of a gene of interest) in a sample. In embodiments, the method includes contacting the sample (e.g., a cell or tissue sample) including the nucleic acid molecule with a polynucleotide probe (e.g., a probe such as a padlock probe). In embodiments, the polynucleotide probe includes the first and second polynucleotide sequences. For example, the polynucleotide probe includes a region that hybridizes to the nucleic acid molecule directly or indirectly, and an optional target barcode region that is useful for identifying the probe. In embodiments, the probe is capable of forming a circular probe when hybridized to the nucleic acid molecule directly or indirectly. In some embodiments, the probe is a linear probe prior to hybridization to the nucleic acid molecule and can be circularized upon binding to the nucleic acid molecule. In embodiments, the linear probe is a padlock probe that hybridizes to an RNA template and is circularized using a ligase as described herein (e.g., a recombinantly produced ligase), and/or a recombinant ligase composition, as described herein). Typically, a padlock probe is a linear DNA polynucleotide where the terminal ends of the probe are complementary to internal sequences of a target molecule of interest. The nature of the complementarity brings the 5′ and 3′-ends of the probe sequence adjacent to each other such that the ends may be ligated to form a circular polynucleotide, which may then be amplified and/or detected.

In embodiments, ligation is a direct ligation. “Direct ligation” means that the ends of the polynucleotides (e.g., the first polynucleotide sequence described herein and/or the second polynucleotide sequence described herein) hybridize immediately adjacently to one another to form a substrate for a ligase enzyme resulting in their ligation to each other (intramolecular ligation). In embodiments, ligation is an indirect ligation. In contrast, “indirect ligation” means that the ends of the polynucleotides (e.g., the first polynucleotide sequence described herein and/or the second polynucleotide sequence described herein) hybridize non-adjacently to one another, e.g., separated by one or more intervening nucleotides or “gaps”. In embodiments, the ends are not ligated directly to each other, but instead occurs either via the intermediacy of one or more intervening (so-called “gap” or “gap-filling” (oligo)nucleotides) or by the extension of the 3′ end of a probe to “fill” the “gap” corresponding to said intervening nucleotides (intermolecular ligation). In some cases, the gap of one or more nucleotides between the hybridized ends of the polynucleotides may be “filled” by one or more “gap” (oligo)nucleotide(s) which are complementary to a splint, padlock probe, or target nucleic acid. The gap may be a gap of 1 to 150 nucleotides, gap of 10 to 100 nucleotides, or a gap of 1 to 40 nucleotides, or a gap of 3 to 40 nucleotides. In embodiments, the gap may be a gap of about 1, 2, 3, 4, 5, 6, 7, 8, 9 or 10 or more nucleotides, of any integer (or range of integers) of nucleotides in between the indicated values. In some embodiments, the gap between the terminal regions may be filled by a gap oligonucleotide or by extending the 3′ end of a polynucleotide. In some cases, ligation involves ligating the ends of the probe to at least one gap (oligo)nucleotide, such that the gap (oligo)nucleotide becomes incorporated into the resulting polynucleotide. In some embodiments, the ligation herein is preceded by gap filling. In other embodiments, the ligation herein does not require gap filling.

In embodiments, ligating the first polynucleotide sequence and the second polynucleotide sequence together includes extending the first polynucleotide sequence and ligating the extended first polynucleotide sequence to the second polynucleotide sequence to generate a ligated product. In embodiments, the ligated product is a circular polynucleotide. In embodiments, the ligated product is a linear polynucleotide.

In embodiments, a reaction vessel may be provided as a well, for example, a well in an array or microplate including 2, 4, 6, 8, 10, 12, 24, 48, 96, 384, or 1536 sample wells. In embodiments, the reaction vessel is a microscope slide (e.g., a glass slide about 75 mm by about 25 mm). In embodiments the slide is a concavity slide (e.g., the slide includes a depression). In embodiments, the slide includes a coating for enhanced cell adhesion (e.g., poly-L-lysine, silanes, carbon nanotubes, polymers, epoxy resins, or gold). Suitable coatings may include, for example, poly-L-lysine, extracellular matrix proteins such as collagen or fibronectin, laminin, Matrigel®, silane-based reagents, carbon nanotubes, synthetic polymers (e.g., polyethyleneimine, poly-D-lysine), epoxy resins, gold, or biofunctionalized hydrogels. In embodiments, the microplate is a flat glass or plastic tray in which an array of wells are formed, wherein each well can hold between from a few microliters to hundreds of microliters of fluid reagents and samples. In embodiments, the reaction vessel includes a microfluidic device. In embodiments, the microfluidic device includes a flow cell described herein. In embodiments, the microfluidic device includes a functionalized tissue slide. In embodiments, the microfluidic device includes an imaging system or detection apparatus. In embodiments, the reaction vessel is a container.

In embodiments, the method further includes amplifying the ligated product by extending an amplification primer hybridized to the ligated product with a polymerase to generate an extension product. In embodiments, the first polynucleotide sequence and the second polynucleotide sequence are part of the same single-stranded polynucleotide sequence, which may be ligated to form a circular polynucleotide. In embodiments, the ligated product is a circular polynucleotide. In embodiments, the method includes amplifying the circular polynucleotide to generate a complement of the circular polynucleotide.

In embodiments, amplifying the ligated product (e.g., a circular polynucleotide) occurs in or on a cell. In embodiments, amplifying the ligated product occurs in a tissue. In embodiments, the ligated product is a circular polynucleotide. In embodiments, the ligated product is a linear polynucleotide. In embodiments, amplifying the circular polynucleotide occurs in a cell. In embodiments, amplifying the circular polynucleotide occurs in a tissue.

In embodiments, the method further includes amplifying a nucleic acid molecule (e.g., a nucleic acid molecule or ligated product in a cell or tissue) to generate amplification products. In embodiments, amplifying includes contacting the polynucleotide (e.g., the circular polynucleotide or linear nucleotide) in a reaction vessel described herein (e.g., a flow cell or microplate) with one or more reagents for amplifying the target polynucleotide (e.g., the ligated product). In embodiments, amplifying includes contacting the polynucleotide in well in an array or microplate described herein with one or more reagents for amplifying the target polynucleotide. Examples of reagents include but are not limited to a polymerase, buffer, and nucleotides (e.g., an amplification reaction mixture). In certain embodiments the term “amplifying” refers to a method that includes a polymerase chain reaction (PCR). Conditions conducive to amplification (i.e., amplification conditions) are known and often include at least a suitable polymerase, a suitable template, a suitable primer or set of primers, suitable nucleotides (e.g., dNTPs), a suitable buffer, and application of suitable annealing, hybridization and/or extension times and temperatures. In embodiments, amplifying generates an amplicon. In embodiments, amplifying generates a colony. In embodiments, an amplicon contains multiple, tandem copies of the circularized nucleic acid molecule of the corresponding sample nucleic acid. The number of copies can be varied by appropriate modification of the amplification reaction including, for example, varying the number of amplification cycles run, using polymerases of varying processivity in the amplification reaction and/or varying the length of time that the amplification reaction is run, as well as modification of other conditions known in the art to influence amplification yield. Generally, the number of copies of a nucleic acid in an amplicon is at least 100, 200, 500, 1000, 2000, 3000, 4000, 5000, 6000, 7000, 8000, 9000 and 10,000 copies, and can be varied depending on the application. As disclosed herein, one form of an amplicon is as a nucleic acid “ball” localized to the particle and/or well of the array. The number of copies of the nucleic acid can therefore provide a desired size of a nucleic acid “ball” or a sufficient number of copies for subsequent analysis of the amplicon, e.g., sequencing.

In embodiments, amplifying includes bridge polymerase chain reaction (bPCR) amplification, solid-phase rolling circle amplification (RCA), solid-phase exponential rolling circle amplification (eRCA), solid-phase recombinase polymerase amplification (RPA), solid-phase helicase dependent amplification (HDA), template walking amplification, or emulsion PCR on particles, or combinations of the methods. In embodiments, amplifying includes a bridge polymerase chain reaction amplification. In embodiments, amplifying includes a thermal bridge polymerase chain reaction (t-bPCR) amplification. In embodiments, amplifying includes a chemical bridge polymerase chain reaction (c-bPCR) amplification. Chemical bridge polymerase chain reactions include fluidically cycling a denaturant (e.g., formamide) and one or more additives (e.g., ethylene glycol) and maintaining the temperature within a narrow temperature range (e.g., +/−5° C.) or isothermally. In embodiments, c-bPCR does not include isothermal amplification, rather it requires minor (e.g., +/−5° C.) thermal oscillations. In contrast, thermal bridge polymerase chain reactions include thermally cycling between high temperatures (e.g., 85° C.-95° C.) and low temperatures (e.g., 60° C.-70° C.). Thermal bridge polymerase chain reactions may also include a denaturant, typically at a much lower concentration than traditional chemical bridge polymerase chain reactions. In embodiments, amplifying includes generating a double-stranded amplification product.

It will be appreciated that any of the amplification methodologies described herein or known in the art can be utilized with universal or target-specific primers to amplify the target polynucleotide. Suitable methods for amplification include, but are not limited to, the polymerase chain reaction (PCR), strand displacement amplification (SDA), transcription mediated amplification (TMA) and nucleic acid sequence-based amplification (NASBA), for example, as described in U.S. Pat. No. 8,003,354, which is incorporated herein by reference in its entirety. The above amplification methods can be employed to amplify one or more nucleic acids of interest. Additional examples of amplification processes include, but are not limited to, bridge-PCR, recombinase polymerase amplification (RPA), loop-mediated isothermal amplification (LAMP), rolling circle amplification (RCA), strand displacement amplification (SDA), rolling circle amplification (RCA) with exponential strand displacement amplification. In embodiments, amplification includes an isothermal amplification reaction. In embodiments, amplification includes bridge amplification. In general, bridge amplification uses repeated steps of annealing of primers to templates, primer extension, and separation of extended primers from templates. Because primers are attached within the core polymer, the extension products released upon separation from an initial template is also attached within the core. The 3′ end of an amplification product is then permitted to anneal to a nearby reverse primer that is also attached within the core, forming a “bridge” structure. The reverse primer is then extended to produce a further template molecule that can form another bridge. In embodiments, forward and reverse primers hybridize to primer binding sites that are specific to a particular target nucleic acid. In embodiments, forward and reverse primers hybridize to primer binding sites that have been added to, and are common among, target polynucleotides. Adding a primer binding site to target nucleic acids can be accomplished by any suitable method, examples of which include the use of random primers having common 5′ sequences and ligating adapter nucleotides that include the primer binding site. Examples of additional clonal amplification techniques include, but are not limited to, bridge PCR, solid-phase rolling circle amplification (RCA), solid-phase exponential rolling circle amplification, solid-phase recombinase polymerase amplification (RPA), solid-phase helicase dependent amplification (HDA), template walking amplification, emulsion PCR on particles (beads), or combinations of the aforementioned methods. Optionally, during clonal amplification, additional solution-phase primers can be supplemented in the microplate for enabling or accelerating amplification. In embodiments, the amplifying includes rolling circle amplification (RCA) or rolling circle transcription (RCT) (see, e.g., Lizardi et al., Nat. Genet. 19:225-232 (1998), which is incorporated herein by reference in its entirety). Several suitable rolling circle amplification methods are known in the art. For example, RCA amplifies a circular polynucleotide (e.g., DNA) by polymerase extension of an amplification primer complementary to a portion of the template polynucleotide. This process generates copies of the circular polynucleotide template such that multiple complements of the template sequence arranged end to end in tandem are generated (i.e., a concatemer) locally preserved at the site of the circle formation. In embodiments, the amplifying occurs at isothermal conditions. In embodiments, the amplifying includes hybridization chain reaction (HCR). HCR uses a pair of complementary, kinetically trapped hairpin oligomers to propagate a chain reaction of hybridization events, as described in Dirks, R. M., & Pierce, N. A. (2004) PNAS USA, 101(43), 15275-15278, which is incorporated herein by reference for all purposes. In embodiments, the amplifying includes branched rolling circle amplification (BRCA); e.g., as described in Fan T, Mao Y, Sun Q, et al. Cancer Sci. 2018; 109:2897-2906, which is incorporated herein by reference in its entirety. In embodiments, the amplifying includes hyberbranched rolling circle amplification (HRCA). Hyperbranched RCA uses a second primer complementary to the first amplification product. This allows products to be replicated by a strand-displacement mechanism, which yields drastic amplification within an isothermal reaction (Lage et al., Genome Research 13:294-307 (2003), which is incorporated herein by reference in its entirety). In embodiments, amplifying includes polymerase extension of an amplification primer. In embodiments, the polymerase is T4, T7, Sequenase, Taq, Klenow, and Pol I DNA polymerases. SD polymerase, Bst large fragment polymerase, or a phi29 polymerase or mutant thereof. In embodiments, the strand-displacing enzyme is an SD polymerase, Bst large fragment polymerase, or a phi29 polymerase or mutant thereof. In embodiments, the strand-displacing polymerase is phi29 polymerase, phi29 mutant polymerase or a thermostable phi29 mutant polymerase. A “phi polymerase” (or “D29 polymerase”) is a DNA polymerase from the (29 phage or from one of the related phages that, like D29, contain a terminal protein used in the initiation of DNA replication. For example, phi29 polymerases include the B103, GA-1, PZA, Φ15, BS32, M2Y (also known as M2), Nf, G1, Cp-1, PRD1, PZE, SFS, Cp-5, Cp-7, PR4, PR5, PR722, L17, Φ21, and AV-1 DNA polymerases, as well as chimeras thereof. A phi29 mutant DNA polymerase includes one or more mutations relative to naturally-occurring wild-type phi29 DNA polymerases (see, e.g., WO 2024/076991, which is incorporated herein by reference for all purposes), for example, one or more mutations that alter interaction with and/or incorporation of nucleotide analogs, increase stability, increase read length, enhance accuracy, increase phototolerance, and/or alter another polymerase property, and can include additional alterations or modifications over the wild-type phi29 DNA polymerase, such as one or more deletions, insertions, and/or fusions of additional peptide or protein sequences. Thermostable phi29 mutant polymerases are known in the art, see for example US 2014/0322759, which is incorporated herein by reference for all purposes. For example, a thermostable phi29 mutant polymerase refers to an isolated bacteriophage phi29 DNA polymerase including at least one mutation selected from the group consisting of M8R, V51A, M97T, L123S, G197D, K209E, E221K, E239G, Q497P, K512E, E515A, and F526 (relative to wild type phi29 polymerase). In embodiments, the polymerase is a phage or bacterial RNA polymerases (RNAPs). In embodiments, the polymerase is a T7 RNA polymerase. In embodiments, the polymerase is an RNA polymerase. Useful RNA polymerases include, but are not limited to, viral RNA polymerases such as T7 RNA polymerase, T3 polymerase, SP6 polymerase, and Kll polymerase; Eukaryotic RNA polymerases such as RNA polymerase I, RNA polymerase II, RNA polymerase III, RNA polymerase IV, and RNA polymerase V; and Archaea RNA polymerase.

In embodiments, the method further includes detecting the amplification products. In embodiments, detecting the amplification products includes detecting a label (e.g., a labeled oligonucleotide bound to an amplification product or a labeled nucleotide bound to a primer bound to the amplification product). In embodiments, detecting the amplification products includes detecting the label of a fluorescently labeled oligonucleotide. In embodiments, detecting includes sequencing. In embodiments, sequencing includes extending a sequencing primer annealed to the amplification product to incorporate a nucleotide containing a detectable label that indicates the identity of a nucleotide in the amplification product, detecting the detectable label, and optionally repeating the extending and detecting of steps. In embodiments, the methods include sequencing one or more bases of a target nucleic acid (e.g., amplification product) by extending a sequencing primer hybridized to the target nucleic acid (e.g., an amplification product of a target nucleic acid). In embodiments, the sequencing includes sequencing-by-synthesis, sequencing-by-binding, sequencing by ligation, sequencing-by-hybridization, or pyrosequencing, and generates a sequencing read. In embodiments, generating a sequencing read includes executing a plurality of sequencing cycles, each cycle including extending the sequencing primer by incorporating a nucleotide or nucleotide analogue using a polymerase and detecting a characteristic signature indicating that the nucleotide or nucleotide analogue has been incorporated.

In embodiments, sequencing includes a plurality of sequencing cycles. In embodiments, sequencing includes 20 to 100 sequencing cycles. In embodiments, sequencing includes 50 to 100 sequencing cycles. In embodiments, sequencing includes 50 to 300 sequencing cycles. In embodiments, sequencing includes 50 to 150 sequencing cycles. In embodiments, sequencing includes at least 10, 20, 30 40, or 50 sequencing cycles. In embodiments, sequencing includes at least 10 sequencing cycles. In embodiments, sequencing includes 10 to 20 sequencing cycles. In embodiments, sequencing includes 10, 11, 12, 13, 14, or 15 sequencing cycles. In embodiments, sequencing includes (a) extending a sequencing primer by incorporating a labeled nucleotide, or labeled nucleotide analogue and (b) detecting the label to generate a signal for each incorporated nucleotide or nucleotide analogue.

In embodiments, the method includes sequencing the first and/or the second strand of a amplification product by extending a sequencing primer hybridized thereto. A variety of sequencing methodologies can be used such as sequencing-by-synthesis (SBS), pyrosequencing, sequencing by ligation (SBL), or sequencing by hybridization (SBH). Pyrosequencing detects the release of inorganic pyrophosphate (PPi) as particular nucleotides are incorporated into a nascent nucleic acid strand (Ronaghi, et al., Analytical Biochemistry 242(1), 84-9 (1996); Ronaghi, Genome Res. 11(1), 3-11 (2001); Ronaghi et al. Science 281(5375), 363 (1998); U.S. Pat. Nos. 6,210,891; 6,258,568; and. 6,274,320, each of which is incorporated herein by reference in its entirety). In pyrosequencing, released PPi can be detected by being converted to adenosine triphosphate (ATP) by ATP sulfurylase, and the level of ATP generated can be detected via light produced by luciferase. In this manner, the sequencing reaction can be monitored via a luminescence detection system. In both SBL and SBH methods, target nucleic acids, and amplicons thereof, that are present at features of an array are subjected to repeated cycles of oligonucleotide delivery and detection. SBL methods, include those described in Shendure et al. Science 309:1728-1732 (2005); U.S. Pat. Nos. 5,599,675; and 5,750,341, each of which is incorporated herein by reference in its entirety; and the SBH methodologies are as described in Bains et al., Journal of Theoretical Biology 135(3), 303-7 (1988); Drmanac et al., Nature Biotechnology 16, 54-58 (1998); Fodor et al., Science 251(4995), 767-773 (1995); and WO 1989/10977, each of which is incorporated herein by reference in its entirety.

In SBS, extension of a nucleic acid primer along a nucleic acid template is monitored to determine the sequence of nucleotides in the template. The underlying chemical process can be catalyzed by a polymerase, wherein fluorescently labeled nucleotides are added to a primer (thereby extending the primer) in a template dependent fashion such that detection of the order and type of nucleotides added to the primer can be used to determine the sequence of the template. A plurality of different nucleic acid fragments can be subjected to an SBS technique under conditions where events occurring for different templates can be distinguished due to their location in the array. In embodiments, the sequencing step includes annealing and extending a sequencing primer to incorporate a detectable label that indicates the identity of a nucleotide in the target polynucleotide, detecting the detectable label, and repeating the extending and detecting steps. In embodiments, the methods include sequencing one or more bases of a target nucleic acid by extending a sequencing primer hybridized to a target nucleic acid (e.g., an amplification product produced by the amplification methods described herein). In embodiments, the sequencing step may be accomplished by an SBS process. In embodiments, sequencing includes a sequencing by synthesis process, where individual nucleotides are identified iteratively, as they are polymerized to form a growing complementary strand. In embodiments, nucleotides added to a growing complementary strand include both a label and a reversible chain terminator that prevents further extension, such that the nucleotide may be identified by the label before removing the terminator to add and identify a further nucleotide. Such reversible chain terminators include removable 3′ blocking groups, for example as described in U.S. Pat. Nos. 10,738,072. Once such a modified nucleotide has been incorporated into the growing polynucleotide chain complementary to the region of the template being sequenced, there is no free 3′-OH group available to direct further sequence extension and therefore the polymerase cannot add further nucleotides. Once the identity of the base incorporated into the growing chain has been determined, the 3′ block may be removed to allow addition of the next successive nucleotide. By ordering the products derived using these modified nucleotides it is possible to deduce the DNA sequence of the DNA template. Non-limiting examples of suitable labels are described in U.S. Pat. Nos. 8,178,360, 5,188,934 (4,7-dichlorofluorscein dyes); U.S. Pat. No. 5,366,860 (spectrally resolvable rhodamine dyes); U.S. Pat. No. 5,847,162 (4,7-dichlororhodamine dyes); U.S. Pat. No. 4,318,846 (ether-substituted fluorescein dyes); U.S. Pat. No. 5,800,996 (energy transfer dyes); U.S. Pat. No. 5,066,580 (xanthene dyes): U.S. Pat. No. 5,688,648 (energy transfer dyes); and the like.

Sequencing includes, for example, detecting a sequence of signals. Examples of sequencing include, but are not limited to, sequencing by synthesis (SBS) processes in which reversibly terminated nucleotides carrying fluorescent dyes are incorporated into a growing strand, complementary to the target strand being sequenced. In embodiments, the nucleotides are labeled with up to four unique fluorescent dyes. In embodiments, the nucleotides are labeled with at least two unique fluorescent dyes. In embodiments, the readout is accomplished by epifluorescence imaging. A variety of sequencing chemistries are available, non-limiting examples of which are described herein.

As used herein, “sequencing-by-binding” refers to a sequencing technique wherein specific binding of a polymerase and cognate nucleotide to a primed template nucleic acid molecule (e.g., blocked primed template nucleic acid molecule) is used for identifying the next correct nucleotide to be incorporated into the primer strand of the primed template nucleic acid molecule. The specific binding interaction need not result in chemical incorporation of the nucleotide into the primer. In some embodiments, the specific binding interaction can precede chemical incorporation of the nucleotide into the primer strand or can precede chemical incorporation of an analogous, next correct nucleotide into the primer. Thus, detection of the next correct nucleotide can take place without incorporation of the next correct nucleotide. As used herein, the “next correct nucleotide” (sometimes referred to as the “cognate” nucleotide) is the nucleotide having a base complementary to the base of the next template nucleotide. The next correct nucleotide will hybridize at the 3′-end of a primer to complement the next template nucleotide. The next correct nucleotide can be, but need not necessarily be, capable of being incorporated at the 3′ end of the primer. For example, the next correct nucleotide can be a member of a ternary complex that will complete an incorporation reaction or, alternatively, the next correct nucleotide can be a member of a stabilized ternary complex that does not catalyze an incorporation reaction. A nucleotide having a base that is not complementary to the next template base is referred to as an “incorrect” (or “non-cognate”) nucleotide.

Use of the sequencing method outlined above is a non-limiting example, as essentially any sequencing methodology which relies on successive incorporation of nucleotides into a polynucleotide chain can be used. Suitable alternative techniques include, for example, pyrosequencing methods, FISSEQ (fluorescent in situ sequencing), MPSS (massively parallel signature sequencing), or sequencing by ligation-based methods.

In embodiments, the method further includes obtaining an image of a cell or tissue. In embodiments, the imaging includes phase-contrast microscopy, bright-field microscopy, Nomarski differential-interference-contrast microscopy, dark field microscopy, electron microscopy, or cryo-electron microscopy. In embodiments, the light transmittance of the sample is measured. For example, light transmittance may be measured with a visible near-infrared optical fiber spectrometer, wherein a circular spot of light (e.g., diameter, 5 mm) is irradiated on the central part a sample and the transmitted light is collected using an optical sensor.

In embodiments, the method further includes an imaging modality including immunofluorescence (IF), or immunohistochemistry modality (e.g., immunostaining). In embodiments, the method includes ER staining (e.g., contacting the tissue section with a cell-permeable dye which localizes to the endoplasmic reticula), Golgi staining (e.g., contacting the tissue section with a cell-permeable dye which localizes to the Golgi), F-actin staining (e.g., contacting the tissue section with a phalloidin-conjugated dye that binds to actin filaments), lysosomal staining (e.g., contacting the tissue section with a cell-permeable dye that accumulates in the lysosome via the lysosome pH gradient), mitochondrial staining (e.g., contacting the tissue section with a cell-permeable dye which localizes to the mitochondria), nucleolar staining, or plasma membrane staining. For example, the method includes live cell imaging (e.g., obtaining images of the tissue section) prior to or during fixing, immobilizing, and permeabilizing the tissue section. Immunohistochemistry (IHC) is a powerful technique that exploits the specific binding between an antibody and antigen to detect and localize specific antigens in cells and tissue, commonly detected and examined with the light microscope. Known IHC modalities may be used, such as the protocols described in Magaki, S., Hojat, S. A., Wei, B., So, A., & Yong, W. H. (2019). Methods in molecular biology (Clifton, N.J.), 1897, 289-298, which is incorporated herein by reference. In embodiments, the additional imaging modality includes bright field microscopy, phase contrast microscopy, Nomarski differential-interference-contrast microscopy, or dark field microscopy. In embodiments, the method further includes determining the cell morphology of the tissue section (e.g., the cell boundary or cell shape) using known methods in the art. For example, to determining the cell boundary includes comparing the pixel values of an image to a single intensity threshold, which may be determined quickly using histogram-based approaches as described in Carpenter, A. et al Genome Biology 7, R100 (2006) and Arce, S., Sci Rep 3, 2266 (2013)). By “microscopic analysis” is meant the analysis of a specimen using techniques that provide for the visualization of aspects of a specimen that cannot be seen with the unaided eye, i.e., that are not within the resolution range of the normal human eye. Such techniques may include, without limitation, optical microscopy, e.g., bright field, oblique illumination, dark field, phase contrast, differential interference contrast, interference reflection, epifluorescence, confocal microscopy, CLARITY-optimized light sheet microscopy (COLM), light field microscopy, tissue expansion microscopy, etc., laser microscopy, such as, two photon microscopy, electron microscopy, and scanning probe microscopy. By “preparing a biological specimen for microscopic analysis” is generally meant rendering the specimen suitable for microscopic analysis at an unlimited depth within the specimen. In embodiments, the immobilized tissue section is imaged using “optical sectioning” techniques, such as laser scanning confocal microscopes, laser scanning 2-Photon microscopy, parallelized confocal (i.e. spinning disk), computational image deconvolution methods, and light sheet approaches. Optical sectioning microscopy methods provide information about single planes of a volume by minimizing contributions from other parts of the volume and do so without physical sectioning. The resulting “stack” of such optically sectioned images, represents a full reconstruction of the 3-dimensional features of a tissue volume. A typical confocal microscope includes a 10×/0.5 objective (dry; working distance, 2.0 mm) and/or a 20×/0.8 objective (dry; working distance, 0.55 mm), with a s z-step interval of 1 to 5 m. A typical light sheet fluorescence microscope includes an sCMOS camera, a 2×/0.5 objective lens, and zoom microscope body (magnification range of ×0.63 to ×6.3). For entire scanning of whole samples, the z-step interval is 5 or 10 μm, and for image acquisition in the regions of interest, an interval in the range of 2 to 5 μm may be used.

In embodiments, the collection of information (e.g., sequencing information and cell morphology) is referred to as a signature. The term “signature” may encompass any gene or genes, protein or proteins, or epigenetic element(s) whose expression profile or whose occurrence is associated with a specific cell type, subtype, or cell state of a specific cell type or subtype within a population of cells. It is to be understood that also when referring to proteins (e.g., differentially expressed proteins), such may fall within the definition of “gene” signature. Levels of expression or activity or prevalence may be compared between different cells in order to characterize or identify for instance signatures specific for cell (sub)populations. Increased or decreased expression or activity of signature genes may be compared between different cells in order to characterize or identify for instance specific cell (sub)populations.

In embodiments, the methods described herein may further include constructing a 3-dimensional pattern of abundance, expression, and/or activity of each target from spatial patterns of abundance, expression, and/or activity of each target of multiple samples. In embodiments, the multiple samples can be consecutive tissue sections of a 3-dimensional tissue sample.

In embodiments, the method includes immobilizing a cell or tissue onto a solid support. The cell or tissue may be manipulated prior to immobilizing the cell or tissue onto a solid support using known techniques in the art (see, e.g., PCT Publication WO2023076832A1). In embodiments, the method further includes cutting a sample portion from the biological sample (e.g., including cells or tissues) using a punch device such that the punch device contains the sample portion; mounting the punch device containing the sample portion onto the first solid support as described herein (e.g., inverting the punch device); pushing the sample portion out of the punch device using a piston, so that all or a portion thereof of the sample portion is positioned on the first solid support as described herein. In embodiments, the method further includes cutting a sample portion from the biological sample using two or more punch devices such that each punch device contains a different the sample portion; mounting each punch device containing the sample portion onto the first solid support as described herein; pushing the sample portions out of the punch devices using one or more pistons so that the sample portions are positioned onto the first solid support as described herein.

In embodiments, the cell or tissue includes a biomolecule that is targeted by the first polynucleotide sequence described herein and the second polynucleotide sequence described herein. In embodiments, the biomolecule is a nucleic acid molecule, carbohydrate, or protein. In embodiments, the biomolecule is a nucleic acid molecule. In embodiments, the biomolecule is a carbohydrate. In embodiments, the biomolecule is a protein. The biomolecule to be detected can be any biological molecules including but not limited to proteins, nucleic acids, lipids, carbohydrates, ions, or multicomponent complexes containing any of the above. Examples of subcellular targets include organelles, e.g., mitochondria, Golgi apparatus, endoplasmic reticulum, chloroplasts, endocytic vesicles, exocytic vesicles, vacuoles, lysosomes, etc. Exemplary nucleic acid targets can include genomic DNA of various conformations (e.g., A-DNA, B-DNA, Z-DNA), mitochondria DNA (mtDNA), mRNA, tRNA, rRNA, hRNA, miRNA, and piRNA.

A biomolecule to be detected or a plurality of biomolecules to be detected using the methods described herein can be isolated or obtained from a sample. Alternatively, in embodiments the biomolecule is located in a cell or tissue of a sample. In embodiments, the biomolecule includes a polynucleotide capable of being ligated by a ligase described herein or variant thereof. A sample can be any specimen that is isolated or obtained from a subject or part thereof. A sample can be any specimen that is isolated or obtained from multiple subjects. Non-limiting examples of specimens include fluid or tissue from a subject, including, without limitation, blood or a blood product (e.g., serum, plasma, platelets, buffy coats, or the like), umbilical cord blood, chorionic villi, amniotic fluid, cerebrospinal fluid, spinal fluid, lavage fluid (e.g., lung, gastric, peritoneal, ductal, ear, arthroscopic), a biopsy sample, celocentesis sample, cells (blood cells, lymphocytes, placental cells, stem cells, bone marrow derived cells, embryo or fetal cells) or parts thereof (e.g., mitochondrial, nucleus, extracts, or the like), urine, feces, sputum, saliva, nasal mucous, prostate fluid, lavage, semen, lymphatic fluid, bile, tears, sweat, breast milk, breast fluid, the like or combinations thereof. Non-limiting examples of tissues include organ tissues (e.g., liver, kidney, lung, thymus, adrenals, skin, bladder, reproductive organs, intestine, colon, spleen, brain, the like or parts thereof), epithelial tissue, hair, hair follicles, ducts, canals, bone, eye, nose, mouth, throat, ear, nails, the like, parts thereof or combinations thereof. A sample may include cells or tissues that are normal, healthy, diseased (e.g., infected), and/or cancerous (e.g., cancer cells). A sample obtained from a subject may include cells or cellular material (e.g., nucleic acids) of multiple organisms (e.g., virus nucleic acid, fetal nucleic acid, bacterial nucleic acid, parasite nucleic acid). A sample may include a cell and RNA transcripts. A sample can include nucleic acids obtained from one or more subjects. In some embodiments a sample may include nucleic acid obtained from a single subject.

In embodiments, the biomolecule is on the surface of the tissue section or on the surface of the cell. In embodiments, the detection agent includes a protein-specific binding agent. In embodiments, the detection agent includes a protein-specific binding agent bound to a nucleic acid sequence (e.g., a nucleic acid label), bioconjugate reactive moiety, an enzyme, or a fluorophore. In embodiments, the protein-specific binding agent is an antibody, single domain antibody, single-chain Fv fragment (scFv), antibody fragment-antigen binding (Fab), affimer, or an aptamer. In embodiments, the protein-specific binding agent is an antibody. In embodiments, the protein-specific binding agent is a single domain antibody. In embodiments, the protein-specific binding agent is a single-chain Fv fragment (scFv). In embodiments, the protein-specific binding agent is an antibody fragment-antigen binding (Fab). In embodiments, the protein-specific binding agent is an affimer. In embodiments, the protein-specific binding agent is an aptamer.

In embodiments, density of polynucleotides on the solid support may be tuned. For example, in embodiments, the solid support includes a density of at least about 100 polynucleotides per mm², about 1,000 polynucleotides per mm², about 0.1 million polynucleotides per mm², about 1 million polynucleotides per mm², about 2 million polynucleotides per mm², about 5 million polynucleotides per mm², about 10 million polynucleotides per mm², about 50 million polynucleotides per mm², or more. In embodiments, the solid support includes no more than about 50 million polynucleotides per mm², about 10 million polynucleotides per mm², about 5 million polynucleotides per mm², about 2 million polynucleotides per mm², about 1 million polynucleotides per mm², about 0.1 million polynucleotides per mm², about 1,000 polynucleotides per mm², about 100 polynucleotides per mm², or less. In embodiments, the solid support includes about 500, 1,000, 2,500, 5,000, or about 25,000 polynucleotides per mm². In embodiments, the solid support includes about 1×10⁶ to about 1×10¹² polynucleotides. In embodiments, the solid support includes about 1×10⁷ to about 1×10¹² polynucleotides. In embodiments, the solid support includes about 1×10⁸ to about 1×10¹² polynucleotides. In embodiments, the solid support includes about 1×10⁶ to about 1×10⁹ polynucleotides. In embodiments, the solid support includes about 1×10⁹ to about 1×10¹⁰ polynucleotides. In embodiments, the solid support includes about 1×107 to about 1×109 polynucleotides. In embodiments, the solid support includes about 1×10⁸ to about 1×10⁹ polynucleotides. In embodiments, the solid support includes about 1×10⁶ to about 1×10⁸ polynucleotides. In embodiments, the solid support includes about 1×10⁶, 1×10⁷, 1×10⁸, 1×10⁹, 1×10¹⁰, 1×10¹¹, 1×10¹², 5×10¹², or more polynucleotides. In embodiments, the solid support includes about 1.8×10⁹, 3.7×10⁹, 9.4×10⁹, 1.9×10¹⁰, or about 9.4×10¹⁰ polynucleotides. In embodiments, the solid support includes about 1×10⁶ or more polynucleotides. In embodiments, the solid support includes about 1×10⁷ or more polynucleotides. In embodiments, the solid support includes about 1×10⁸ or more polynucleotides. In embodiments, the solid support includes about 1×10⁹ or more polynucleotides. In embodiments, the solid support includes about 1×10¹⁰ or more polynucleotides. In embodiments, the solid support includes about 1×10¹¹ or more polynucleotides. In embodiments, the solid support includes about 1×10¹² or more polynucleotides. In embodiments, the solid support is a glass slide. In embodiments, the solid support is a about 75 mm by about 25 mm. In embodiments, the solid support includes one, two, three, or four channels.

In embodiments, the solid support includes a polymer layer. In embodiments, the polymer layer includes polymerized units of alkoxysilyl methacrylate, alkoxysilyl acrylate, alkoxysilyl methylacrylamide, alkoxysilyl methylacrylamide, or a copolymer thereof. In embodiments, the polymer layer includes polymerized units of alkoxysilyl methacrylate. In embodiments, the polymer layer includes polymerized units of alkoxysilyl acrylate. In embodiments, the polymer layer includes polymerized units of alkoxysilyl methylacrylamide. In embodiments, the polymer layer includes polymerized units of alkoxysilyl methylacrylamide. In embodiments, the polymer layer includes glycidyloxypropyl-trimethyloxysilane. In embodiments, the polymer layer includes methacryloxypropyl-trimethoxysilane. In embodiments, the polymer layer includes polymerized units of

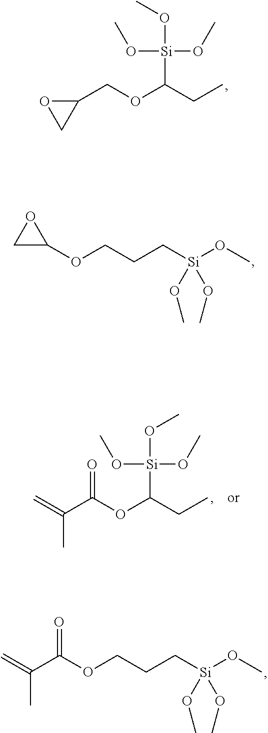

or a copolymer thereof.

In embodiments, the solid support includes a photoresist, alternatively referred to herein as a resist. A “resist” as used herein is used in accordance with its ordinary meaning in the art of lithography and refers to a polymer matrix (e.g., a polymer network). In embodiments, the photoresist is a silsesquioxane resist, an epoxy-based polymer resist, poly(vinylpyrrolidone-vinyl acrylic acid) copolymer resist, an Off-stoichiometry thiol-enes (OSTE) resist, amorphous fluoropolymer resist, a crystalline fluoropolymer resist, polysiloxane resist, or a organically modified ceramic polymer resist. In embodiments, the photoresist is a silsesquioxane resist. In embodiments, the photoresist is an epoxy-based polymer resist. In embodiments, the photoresist is a poly(vinylpyrrolidone-vinyl acrylic acid) copolymer resist. In embodiments, the photoresist is an Off-stoichiometry thiol-enes (OSTE) resist. In embodiments, the photoresist is an amorphous fluoropolymer resist. In embodiments, the photoresist is a crystalline fluoropolymer resist. In embodiments, the photoresist is a polysiloxane resist. In embodiments, the photoresist is an organically modified ceramic polymer resist. In embodiments, the photoresist includes polymerized alkoxysilyl methacrylate polymers and metal oxides (e.g., SiO₂, ZrO, MgO, Al₂O₃, TiO₂ or Ta₂O₅). In embodiments, the photoresist includes polymerized alkoxysilyl acrylate polymers and metal oxides (e.g., SiO₂, ZrO, MgO, Al₂O₃, TiO₂ or Ta₂O₅). In embodiments, the photoresist includes metal atoms, such as Si, Zr, Mg, Al, Ti or Ta atoms.

In embodiments, the solid support is generated by pressing a transparent mold possessing the pattern of interest (e.g., the pattern of wells) into photo-curable liquid film, followed by solidifying the liquid materials via a UV light irradiation. Typical UV-curable resists have low viscosity, low surface tension, and suitable adhesion to the glass substrate. For example, the solid support surface is coated in an organically modified ceramic polymer (ORMOCER®, registered trademark of Fraunhofer-Gesellschaft zur Förderung der angewandten Forschung e. V. in Germany). Organically modified ceramics contain organic side chains attached to an inorganic siloxane backbone. Several ORMOCER® polymers are now provided under names such as “Ormocore”, “Ormoclad” and “Ormocomp” by Micro Resist Technology GmbH. In embodiments, the solid support includes a resist as described in Haas et al Volume 351, Issues 1-2, 30 Aug. 1999, Pages 198-203, US 2015/0079351A1, US 2008/0000373, US 2010/0160478, or U.S. Pat. No. 10,268,096 B2, each of which is incorporated herein by reference. In embodiments, the solid support surface is coated in an organically modified ceramic polymer including (ORMOCER®, registered trademark of Fraunhofer-Gesellschaft zur Förderung der angewandten Forschung e. V. in Germany). In embodiments, the solid support surface is coated in an organically modified ceramic polymer wherein the organically modified ceramic polymer includes an inorganic-organic hybrid polymer that includes Si—O bonds. In embodiments, the solid support surface is coated in an organically modified ceramic polymer wherein the organically modified ceramic polymer includes an inorganic-organic hybrid polymer that includes Si—C bonds. In embodiments, the solid support surface is coated in an organically modified ceramic polymer wherein the organically modified ceramic polymer includes free acrylate moieties. In embodiments, the polymer is an organically modified ceramic polymer wherein the organically modified ceramic polymer includes an inorganic-organic hybrid polymer that includes Si—O bonds. In embodiments, polymer is an organically modified ceramic polymer wherein the organically modified ceramic polymer includes an inorganic-organic hybrid polymer that includes Si—C bonds. In embodiments, the polymer is an organically modified ceramic polymer wherein the organically modified ceramic polymer includes free acrylate moieties. In embodiments, the polymer contains organically crosslinked heteropolysiloxane moieties.

In embodiments, the polymer is attached to a coupling agent. In embodiments, the coupling agent includes a hydrophilic cationic compound. In embodiments, the coupling agent includes (3-aminopropyl)triethoxysilane (APTES), (3-Aminopropyl)trimethoxysilane (APTMS), 7-Aminopropylsilatrane (APS), N-(6-aminohexyl)aminomethyltriethoxysilane (AHAMTES), polyethylenimine (PEI), 5,6-epoxyhexyltriethoxysilane, 3-(trimethoxysilyl)propyl methacrylate (MAPTMS), or triethoxysilylbutyraldehyde, or a combination thereof. In embodiments, the coupling agent includes N-(2-aminoethyl)-3-aminopropyltriethoxysilane (AEAPTES), N-(2-aminoethyl)-3-aminopropyltrimethoxysilane (AEAPTMS), N-(6-aminohexyl) aminomethyltriethoxysilane (AHAMTES), 3-aminopropyldimethylethoxysilane (APDMES), 3-mercaptopropyltrimethoxysilane (MPTMS), glycidyloxypropyl-trimethoxysilane (GOPS), as described by Sypabekova et al. (Biosensors (Basel). 2022 Dec. 27; 13(1):36), or a combination thereof. In embodiments, the coupling agent includes polyethylenimine (PEI). In embodiments, the coupling agent includes branched polyethylenimine (bPEI). In embodiments, the coupling agent includes unbranched polyethylenimine. In embodiments, the coupling agent includes polyethylenimine with an average molecular weight (M_{w}) of about 600, about 800, about 1,300, about 2,000, about 25,000, or about 750,000. In embodiments, the coupling agent includes polyethylenimine with number average molecular weight (Mₙ) of about 600, about 1,300, about 2,100, or about 10,000. In embodiments, the coupling agent includes polyallylamine, poly(ethylene glycol) diamine, (PEG)₃₂ diamine, (PEG)₃ diamine, ethylene diamine, chitosan, polydiallyldimethylammonium chloride (commonly referred as polyDADMAC or polyDDA), triethoxysilylbutyraldehyde (TESBA), 1,5,6-epoxyhexyltriethoxysilane (EHTES), bis(2-hydroxyethyl)-3-aminopropyltriethoxysilane (BHEAPTES), poly-l-lysine (PLL), or spermidine. In embodiments, the coupling agent includes a combination of triethoxysilylbutyraldehyde (TESBA) and polyethylenimine (PEI) or a combination of triethoxysilylbutyraldehyde (TESBA) and chitosan. In embodiments, the coupling agent includes a hydrophilic compound. In embodiments, the coupling agent includes a hydrophilic cationic compound.

The arrays and solid supports for some embodiments have at least one surface located within a flow cell. Flow cells provide a convenient format for housing an array of clusters produced by the methods described herein, in particular when subjected to an SBS or other detection technique that involves repeated delivery of reagents in cycles. In embodiments, the solid support is a multiwell container or an unpatterned solid support (e.g., an unpatterned surface). In embodiments, the solid support is a glass slide including a polymer coating (e.g., a hydrophilic polymer coating). In embodiments, the polymer coating includes a plurality of immobilized oligonucleotides (e.g., an oligonucleotide complementary to the platform primer binding sequence of the adapter). In embodiments, the solid support includes a tissue section.

### EXAMPLES

### Example 1. Identification of Ligase Homologs

The superfamily of ATP-dependent ligases catalyze reactions that are critical for DNA replication, DNA repair, and recombination (Martin et al. Genome Biol. 2002; 3(4): REVIEWS3005). Members of the ATP-dependent ligases family include DNA ligase I, IIIα, IIIβ, IV, provirus ligases, PBCV-1 DNA ligase, and archaeal ligases. Among these ligases, PBCV-1 DNA ligase (also referred to as Chlorella virus DNA ligase and commercially available as SplintR®) is particularly important as PBCV-1 DNA ligase has been shown to have a high selectivity for nicked substrates over substrates with a 1 nucleotide or 2 nucleotide gap (Sriskanda et al. Nucleic Acids Res. 1998 Jan. 15; 26(2): 525-531). Additionally, PBCV-1 DNA ligase is capable to efficiently ligate two DNA templates splinted by a complementary RNA molecule, and such ability of PBCV-1 DNA ligase has been reported to be 100× faster than the ability of T4 DNA ligase or T4 RNA ligase (Jin et al. Nucleic Acids Res. 2016 Jul. 27; 44(13):e116). PBCV-1 DNA ligase and the members of the ATP-dependent ligase family are characterized by the presence of a catalytic domain that includes six structurally conserved motifs: I, III, IIIa, IV, V, and IV, where motif I includes a highly conserved lysine residue that is instrumental for initiating the ligation reaction in the active site of this ligase family (see, e.g., Martin et al. Genome Biol. 2002; 3(4): REVIEWS3005). The lysine residue is located within the highly conserved sequence, KxDGxR, within motif I of the ATP-dependent ligases, where x shown in the conserved sequence, KxDGxR, signifies a variable residue between members of the ATP-dependent ligases family (Sriskanda et al. Nucleic Acids Res. 1998 Jan. 15; 26(2): 525-531).

ATP-dependent ligases catalyze the joining of the 5′-phosphorus-terminated strand with the 3′-hydroxyl-terminated strand in a polynucleotide. For PBCV-1 DNA ligase to initiate ligation of a nicked substrate, the ligation reaction commences with a nucleophilic attack on the α-phosphate of ATP by the ε-amino group of Lys27 to form the ligase-adenylate intermediate, while displacing the pyrophosphate moiety (Sriskanda et al. Nucleic Acids Res. 1998 Jan. 15; 26(2): 525-531). Following the formation of the ligase-adenylate intermediate, the second step of the ligation reaction relies on the residue, D65, to facilitate the transfer of AMP from the active site of the ligase to the 5′-phosphorus terminus of the nicked DNA substrate to generate a DNA-adenylate intermediate (Sriskanda et al. Nucleic Acids Res. 1998 Jan. 15; 26(2): 525-531). The ligation reaction completes with a nucleophilic attack of the DNA-adenylate intermediate by the 3′-hydroxyl-terminated strand, which joins the two strands while releasing AMP (Samai et al. J Biol Chem. 2012 Aug. 17; 287(34):28609-18). Conserved residue R32 in the catalytic domain of PBCV-1 DNA ligase has been shown to be critical for the phosphodiester bond formation in the last step of the ligation reaction (Sriskanda et al. Nucleic Acids Res. 1998 Jan. 15; 26(2): 525-531).

The ability of PBCV-1 DNA ligase to ligate nicked substrate is particularly important for in situ sequencing applications, which often use PBCV-1 DNA ligase to ligate targeted padlock probes in a sample (see, e.g., U.S. Pat. No. 11,492,662 and/or Chen et al. Nucleic Acids Res. 2018 Feb. 28; 46(4):e22). However, challenges derived from the thermostability of PBCV-1 DNA ligase limit its utility and efficiency in various workflows. For example, the lack of thermostability and thermotolerance limits the utility of PBCV-1 DNA ligase to a narrow range of 4° C. to about 30° C., where 25° C. is regarded as the optimal working temperature. Furthermore, the lack of thermostability and thermotolerance causes PBCV-1 DNA ligase to aggregate at high temperatures as described infra and high concentration (e.g., 16 μM). As such, a ligase with high thermostability and thermotolerance to a diverse range of experimental conditions is needed to improve its utility in various in situ workflows.

Described herein are methods directed to using ligating two polynucleotide sequences splinted to a complementary RNA molecule using a thermostable ligase. The identification of the thermostable ligase as described herein was initiated by querying the amino acid sequence of wild type of PBCV-1 DNA ligase (SEQ ID NO:16) using a sequence alignment database (e.g., NCBI Protein BLAST®). Homologs of wild type of PBCV-1 DNA ligase with at least 50% of sequence identity, high coverage, and low E value relative to SEQ ID NO:16 were selected as initial candidates. Structures of the initial ligase candidates were predicted using a protein structure database (e.g., AlphaFold®), and predicted structures were superimposed onto a crystal structure of PBCV-1 DNA ligase (see, e.g., PDB: 2Q2T). The superimposition of two ligase homologs, PCBV NYs1 ligase (SEQ ID NO:1; alternatively referred herein as PB-1) and Blyttiomyces helicus ligase (SEQ ID NO:17; alternatively referred herein as BH-1), exhibited high structural similarity with the structure of wild type PBCV-1 DNA ligase (SEQ ID NO:16). PB-1 (SEQ ID NO:1) exhibits an 83% sequence identity to wild type of PBCV-1 DNA ligase. BH-1 (SEQ ID NO: 17) exhibits an 50% sequence identity to wild type of PBCV-1 DNA ligase. These two homologs were selected for thermostability studies described infra. The ligases identified herein are referred internally with the prefix “PB” and are numbered sequentially. The prefix “PB” was selected as an abbreviation for the chlorovirus host, Paramecium busaria (see, e.g., Van Etten et al. Viruses. 2020 January; 12(1): 20). The ligase variants derived from BH-1 are abbreviated with the prefix “BH” and are numbered sequentially.

Synthesis of PB-1: The cDNA sequence for His-tag PB-1 is provided in SEQ ID NO:18 and was purchased from ATUM. The cDNA sequence of PB-1 was inserted into a kanamycin-resistance gene harboring pD451-SR plasmid, which also harbored a T7 promoter, strong ribosomal binding site, and has a high plasmid copy number. Subsequently, the pD451-SR plasmid was transfected into E. coli and incubated at 37° C. with shaking in 2.0 L flasks until an OD₆₀₀ of 0.6 was reached. Then, isopropyl β-d-1-thiogalactopyranoside (IPTG) (1 mM final concentration) was added to induce specific protein expression. The cells were incubated for 3 hours at 37° C. and collected by centrifugation at 5000 rpm for 5 minutes. Cell pellets were stored at minus 80° C.

All purification steps were performed at 4° C. except as indicated. The frozen cell paste was resuspended in lysis buffer (20 mM Tris pH 7.5, 500 mM NaCl, 1 mM EDTA, 1 mM DTT, 10% glycerol, 1 mM PMSF, and protease inhibitor cocktail, EDTA-free (Thermo Fisher). The suspended cells were subjected to sonication and centrifuged at 20,000 rpm for 20 minutes. Then, the supernatant was heated to 80° C. for 30 min to inactivate host proteins. The mixture was again centrifuged at 20,000 rpm for 20 min and the pellet discarded.

The supernatant was collected and poly(ethyleneimine) was added slowly to the supernatant to a final concentration of 0.4% with continued stirring for 30 minutes. The mixture was centrifuged at 20,000 rpm for 20 min and the pellet was discarded. Then, solid ammonium sulfate was added to the supernatant to 65% saturation with stirring continued for 30 minutes. The mixture was centrifuged at 20,000 rpm for 20 minutes and the precipitated proteins were resuspended in 10 ml Buffer A (20 mM Tris pH 7.0, 50 mM KCl, 0.1 mM EDTA, 10% glycerol, 1 mM DTT, 1 mM PMSF). The protein was dialyzed overnight against 1 L of Buffer A. The dialyzed sample was centrifuged at 20,000 rpm for 20 minutes to remove any precipitate and the supernatant was loaded onto a 5 ml Hi-Trap SP FF column (GE Healthcare) equilibrated with Buffer A. The ligase was eluted using a 100 ml gradient from 50 to 800 mM KCl. Peak fractions were pooled and dialyzed overnight against Buffer B (20 mM Tris pH 7.5, 50 mM KCl, 0.1 mM EDTA, 10% glycerol, 1 mM DTT, 1 mM PMSF). The dialyzed sample was centrifuged at 20,000 rpm for 20 minutes to remove any precipitate and the supernatant was loaded onto a 5 mL Hi-Trap Heparin column (GE Healthcare) equilibrated with Buffer B. The ligase was eluted using a 100 mL gradient from 50 to 800 mM KCl. Peak fractions were pooled and dialyzed overnight against storage buffer (20 mM Tris pH 7.5, 100 mM KCl, 0.1 mM EDTA, 50% glycerol, 1 mM DTT) and stored at −20° C. Following purification, quality control assays were performed to confirm absence of contamination from E. coli exonuclease I, E. coli exonuclease III, E. coli genomic DNA, RNAses. The resultant ligase was utilized in studies to evaluate the thermostability and ability to perform in situ ligation of two ssDNA splinted to a complementary RNA molecule as well as for the development of ligase variants as described infra. Additionally, the resultant ligase was used as the “parent ligase” for the development of the ligase variants described infra. All variants synthesized and/or purified are identified in the Ligase Table.

| Ligase Table. Collection of ligase variants synthesized according to the methods | |
|---|---|
| described herein. As described in other sections of this manuscript, the PB-1 ligase (SEQ ID | |
| NO: 1) was used as the parental template for the design and generation of point mutations. A | |
| person of ordinary skill in the art would recognize the relationship between SEQ ID NO: 1 and | |
| SEQ ID NO: 19. Specifically, SEQ ID NO: 1 represents the amino acid sequence of the wild-type | |
| Paramecium bursaria Chlorella virus 1 (PBCV-1) NYs1 DNA ligase, herein referred to as PB-1. | |
| In contrast, SEQ ID NO: 19 corresponds to the amino acid sequence of the same wild-type ligase, | |
| but with the addition of an N-terminal polyhistidine (His) tag and linker, and is herein referred to | |
| as PB-1 HT. Due to the presence of the 10-residue His-tag and linker at the N-terminus of PB- | |
| 1 HT, all internal amino acid positions in SEQ ID NO: 19 are shifted by +10 relative to their | |
| corresponding positions in SEQ ID NO: 1. For example, amino acid position 244 in SEQ ID | |
| NO: 1 corresponds to position 254 in SEQ ID NO: 19. | |
| Internal Ref | Mutations |
| PB-1 | wild-type |
| PB-35 | V244K |
| PB-36 | V244R |
| PB-37 | V244E |
| PB-38 | V244D |
| PB-39 | S94E |
| PB-40 | G248E |
| PB-41 | G248P |
| PB-42 | G248D |
| PB-43 | V243R |
| PB-44 | V243K |
| PB-45 | V243E |
| PB-46 | S246E |
| PB-47 | S246K |
| PB-48 | S246R |
| PB-49 | S246P |
| PB-50 | F245R |
| PB-51 | F245K |
| PB-52 | 1290R |
| PB-53 | 1290E |
| PB-54 | 1290K |
| PB-55 | V140K |
| PB-56 | V140R |
| PB-57 | 1198K |
| PB-58 | L215D |
| PB-59 | V244K S246E |
| PB-60 | V244K V140K |
| PB-61 | V244K V243K |
| PB-62 | V244R S246E |
| PB-63 | V244R V140K |
| PB-64 | V244R V243K |
| PB-65 | S246E V140K |
| PB-66 | S246E V243K |
| PB-67 | V140K V243K |
| PB-68 | V244K S246E V140K |
| PB-69 | V244K V243K S246E |
| PB-70 | V244K V243K V140K |
| PB-71 | V244R V140K S246E |
| PB-72 | V244R V243K S246E |
| PB-73 | V244R V243K V140K |
| PB-74 | V243K V140K |
| PB-75 | V244K V243K S246E V140K |
| PB-76 | V244R V243K S246E V140K |
| PB-77 | V244K V243K S246E V140K 1135K |
| PB-78 | V244K V243K S246E V140K 1135D |
| PB-79 | V244K V243K S246E V140K 1135R |
| PB-80 | V244K V243K S246E V140K 1138R |
| PB-81 | V244K V243K S246E V140K 1138K |
| PB-82 | V244K V243K S246E V140K 1138E |
| PB-83 | V244K V243K S246E V140K F286K |
| PB-84 | V244K V243K S246E V140K F286R |
| PB-85 | V244K V243K S246E V140K F286E |

### Example 2. Development of Ligase Variants

The ability of PBCV-1 DNA ligase to join polynucleotide sequences while splinted by a complementary RNA molecule is a highly attractive approach for targeting RNA in situ. Despite its value to the detection of polynucleotides in situ via padlock probes, its low thermostability limits its utility in various workflows, especially those requiring elevated as described supra. Improving the thermostability of PBCV-1 DNA ligase while retaining its capability to ligate two polynucleotide sequences (e.g., two strands of ssDNA) that are hybridized to a complementary RNA molecule is greatly needed to expand its utility in a multitude of in situ detection workflows.

For brevity, amino acid mutation nomenclature is used throughout this application. One having skill in the art would understand the amino acid mutation nomenclature, such that C22S refers to cysteine (single letter code is C), at position 22, is replaced with serine (single letter code S). Likewise, it is understood that when an amino acid mutation nomenclature is used and the terminal amino acid code is missing, e.g., C22, it is understood that no mutation was made relative to the wild type (e.g., wild type PCBV-1 ligase or wild type PBCV-1 NYs1 ligase, alternatively referred herein as PB-1).

Described herein are methods and compositions directed to a ligase with high thermostability (e.g., improved thermostability relative to PBCV-1 DNA ligase) and capable of joining two strands of ssDNA while hybridized to a complementary RNA molecule. As described supra in Example 1, a homolog of PBCV-1 DNA ligase was identified using the amino acid sequence of wild type of PBCV-1 DNA ligase (SEQ ID NO:16) with a sequence alignment database. This homolog, PB-1, was synthesized and used as the parent ligase for the development of ligase variants described infra. Ligation efficiencies of ligase variants described herein are compared to wild type of PBCV-1 DNA ligase and the parent ligase (e.g., PB-1). Strategies considered for the derivation of ligase variants with improved thermostability and ligation processivity relative to a control (e.g., wild type PBCV-1 DNA ligase or PB-1) included (1) mutating cysteine residues to serine residues to reduce aggregation; (2) addition of DNA-binding domains to improve binding to ssDNA substrate; and (3) mutating residues proximate to the catalytic domain of the ligase. Additionally, other strategies contemplated toward the development of variants of PB-1 included introducing point mutations to disrupt hydrogen bonds and/or salt bridges thought to contribute to aggregation of the parent ligase.

Provided herein are novel PBCV NYs1 ligases wherein the cysteines are mutated. As an initial test, Applicant mutated the cysteines at positions 22, 69, and 298 to serine amino acids using the backbone of the parent ligase, PB-1 (SEQ ID NO:1). Table 1 reports the selective mutation of C22S; C69S; C298S; and all three cysteines, C22S, C69S, and C298S. Alternatively, truncation at the C-terminus of PB-1 were explored in combination with mutations to cysteine residues; the amino acid sequence of PB-1 with a C-terminus truncation is provided in SEQ ID NO:2. Variants harboring a C-terminus truncation and/or cysteine mutations are also presented in Table 1. While serine was chosen as an initial mutation, any amino acid that eliminates the ability to form free thiols and does not perturb the stability nor function of the ligase is envisioned (e.g., threonine, methionine, alanine, valine, tyrosine, tryptophan, or arginine).

TABLE 1

| Cysteine positions in this table are mutated and/or truncated relative to the | |
|---|---|
| wild type PB-1 (SEQ ID NO: 1) or PB-1 with a C-terminus truncation at C298 (SEQ ID NO: 2). | |
| The mutations in PB-2, PB-3, PB-4, and PB-6 are relative to the parent ligase, PB-1 (SEQ ID | |
| NO: 1). The mutations in PB-5 and PB-7 are relative to PB-1 with a C-terminus truncation at | |
| C298 (SEQ ID NO: 2). | |
| Ref # | Amino acids |
| PB-2 | C22S |
| PB-3 | C69S |
| PB-4 | C298S |
| PB-5 | Truncate C298 |
| PB-6 | C22S; C69S; C298S |
| PB-7 | C22S; C69S; C298 Truncation |

Also provided herein are novel PBCV NYs1 ligases, where a polynucleotide-binding polypeptide is covalently attached to the backbone of the parent ligase, PB-1 (SEQ ID NO:1). In embodiments, the parent ligase is wild type PBCV NYs1 ligases including a C-terminus truncation at amino acid position 298 in SEQ ID NO:1 (as provided in SEQ ID NO:2). In embodiments, the parent ligase is wild type PBCV NYs1 ligases further includes a His tag and linker at the N-terminus in SEQ ID NO:1 (as provided in SEQ ID NO:19 or SEQ ID NO:20). In embodiments, the polynucleotide-binding polypeptide is covalently attached at the N-terminus of the parent ligase, PB-1. In embodiments, the polynucleotide-binding polypeptide is covalently attached at the C-terminus of the parent ligase, PB-1. Bauer et al. showed that the attachment of a polynucleotide-binding polypeptide, such as Sso7d or zinc finger domain of human DNA ligase III, to PBCV-1 DNA ligase increased its binding affinity to DNA substrates and ligation activity relative to wild type PBCV-1 DNA ligase (Bauer et al. PLoS One. 2017 Dec. 28; 12(12):e0190062). Polynucleotide-binding polypeptides, such as Sso7d or its homolog, Sac7d, have been shown to be highly thermostable, stable up to pH 12, and have a preference to binding to sequences including G/C bases (Kalichuk et al. Sci Rep. 2016 Nov. 17:6:37274). As such, the attachment of a polynucleotide-binding polypeptide to PB-1 was explored as an avenue to derive a robust, thermostable ligase with high binding affinity for RNA:DNA substrates that is capable of ligating two polynucleotides hybridized onto a complementary RNA molecule while improving its stability at temperatures used in situ workflows. Table 2 provides embodiments of polynucleotide-binding polypeptide domains contemplated herein (also see, Kalichuk et al. Sci Rep. 2016 Nov. 17:6:37274). In certain contexts, such attachment may increase binding affinity indiscriminately to all free DNA, potentially reducing substrate specificity.

TABLE 2

| Polynucleotide-binding polynucleotide domains contemplated | |
|---|---|
| for attachment onto C-terminus or N-terminus of PB-1. | |
| Polynucleotide- | |
| Binding Polypeptide | SEQ ID NO: |
| Sso7d | SEQ ID NO: 3 |
| Sac7d | SEQ ID NO: 4 |
| Sac7e | SEQ ID NO: 5 |
| Mse7 | SEQ ID NO: 6 |
| Mcu7 | SEQ ID NO: 7 |
| Aho7a | SEQ ID NO: 8 |
| Aho7b | SEQ ID NO: 9 |
| Aho7c | SEQ ID NO: 10 |
| Sto7 | SEQ ID NO: 11 |
| ssh7b | SEQ ID NO: 12 |
| Sis7a | SEQ ID NO: 13 |
| Sis7b | SEQ ID NO: 14 |
| Ssh7a | SEQ ID NO: 15 |
| HU-alpha | SEQ ID NO: 25 |

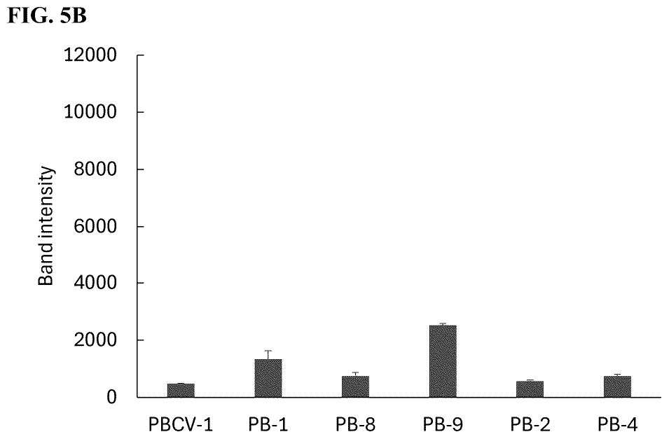

Ligase variant PB-8 includes an Sso7d fused at the C-terminus of SEQ ID NO:1, while PB-9 includes an Sso7d covalently attached to the N-terminus of SEQ ID NO:1. Ligase variant PB-2 includes a C22S point mutation in SEQ ID NO:1, and ligase variant PB-4 includes a C298S point mutation in SEQ ID NO:1. Variants were tested for thermostability at 37° C. (FIG. 5A) and 42° C. (FIG. 5B). 16 uM of ligase was incubated at RT, 30° C., 37° C., and 42° C. for 20 min. Samples were then centrifuged and supernatant was loaded on the gel. Concentration was also measured. Band intensity was quantified using ImageJ. The concentration of PB-8 and PB-9 seems to be consistent from RT to 42° C., suggesting that PB-8 and PB-9 are each thermostable up to 37-42° C. The concentration of PB-2 seems to decrease starting at 37° C., indicating that PB-2 is less thermostable than PB-1 and suggesting the C22S mutation might play a role in the decreased thermostability.

Additional ligase variants were contemplated for the development of a robust ligase, which included those covalently attached to a nucleoid-associated protein HU-alpha polypeptide at the N-terminus. For example, PB-34 is covalently attached to the HU-alpha polypeptide at the N-terminus and includes a C-terminus truncation at amino acid position 298 in SEQ ID NO:1 (SEQ ID NO:23). Ligases other than those with a PBCV NYs1 backbone were considered for covalent attachment to the HU-alpha polypeptide. Using the query of the amino acid sequence of wild type of PBCV-1 DNA ligase (SEQ ID NO:16) with a sequence alignment database (e.g., NCBI Protein BLAST®) as described supra, homologs of wild type of PBCV-1 DNA ligase with at least 50% of sequence identity, high coverage, and low E value relative to SEQ ID NO:16 were selected as initial candidates. Among these homologs was ATCV1_Z187L (SEQ ID NO:26), which exhibited 51% sequence identity to wild type of PBCV-1 DNA ligase. A ligase variant of ATCV1_Z187L, referred herein as AT-1 (SEQ ID NO:24), was synthesized by covalently attaching a His-tag, HU-alpha polypeptide, and linker sequences at the N-terminus of SEQ ID NO:26.

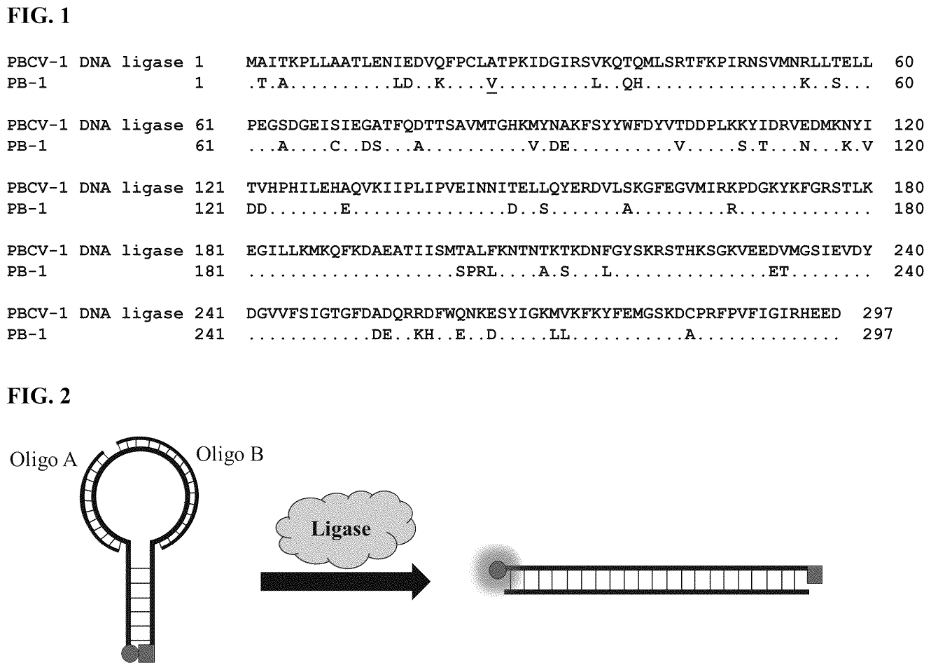

Also provided herein are novel PBCV NYs1 ligases, where point mutations are introduced into the parent ligase of PB-1 with the sequence provided in SEQ ID NO:1 or SEQ ID NO:2. Variants of PB-1 were derived by introducing point mutations to (1) amino acid residues near the conserved sequence, KxDGxR (within motif I) of the catalytic domain, (2) amino acid residues near the bound AMP molecule, and/or (3) amino acid residues near the bound DNA substrate. As an initial study, Applicant leveraged previously reported mutations to amino acid residues in wild type PBCV-1 DNA ligase as a model for deriving variants of PB-1 as wild type PBCV-1 DNA ligase and PB-1 share 83% sequence identity as described supra. FIG. 1 shows an aligned amino acid sequence comparison of PBCV NYs1 ligase, PB-1, bottom, (SEQ ID NO:1) and PBCV-1 Ligase, top, (SEQ ID NO:16). Previously, amino acid residues in nucleotidyl transferase motifs I, such as T25 (Odell et al. Mol Cell. 2000 November; 6(5):1183-93) and L7 (Riballo et al. J Biol Chem. 2001 Aug. 17; 276(33):31124-32); III, such as D65 and E67 (Odell et al. Mol Cell. 2000 November; 6(5):1183-93); IIIa such as F98 (Odell et al. Mol Cell. 2000 November; 6(5):1183-93); motif IV, such as E161 and M164 (Odell et al. Mol Cell. 2000 November; 6(5):1183-93); V, such as K186 and K188 (Odell et al. Mol Cell. 2000 November; 6(5):1183-93); and VI, such as R293 and R297 (Samai et al. J Biol Chem. 2012 Aug. 17; 287(34):28609-18.) in wild type PBCV-1 DNA ligase were disclosed and/or mutated. Additionally, amino acid residues positioned in between the aforementioned nucleotidyl transferase motifs (e.g., R42, positioned between motifs I and III and R176, positioned between motifs IV and V; see Odell et al. Mol Cell. 2000 November; 6(5):1183-93) were also disclosed as critical for the ligation activity of wild type PBCV-1 DNA ligase. In embodiments, the ligase described herein further includes one or more mutations at amino acid position corresponding to position 7, 25, 27, 29, 30, 32, 42, 65, 67, 98, 161, 164, 172, 176, 186, 188, 228, 234, 285, 293, and/or 297 of SEQ ID NO: 1. In embodiments, the ligase described herein further includes one or more mutations at amino acid position corresponding to position 8, 10, 23, 24, 75, 92, 93, 94, 95, 96, 97, 99, 100, 107, 108, 109, 110, 111, 112, 113, 114, 115, 116, 117, 118, 119, 165, 166, 167, 171, 173, 174, 175, 190, 276, and/or 286 of SEQ ID NO:1. In embodiments, the ligase described herein further includes a tryptophan, phenylalanine, tyrosine, glutamic acid, or aspartic acid at the amino acid position corresponding to position 164 of SEQ ID NO:1. In embodiments, the ligase described herein further includes a tryptophan, phenylalanine, or histidine at the amino acid position corresponding to position 7 of SEQ ID NO:1. Point mutations described herein may improve electrostatic interaction between the peptide backbone of the amino acid sequence of the ligase variants described herein and/or between the ligase variants described herein and the DNA substrate. Additionally, the point mutations described herein may enhance the stability achieved from optimizing π stacking or π-cation interactions, which require the interacting groups to be within 6 Å from each other and assume an “en face” geometry between the interacting moieties (see, e.g., Infield et al. J Mol Biol. 2021 Aug. 20; 433(17): 167035). Variants were synthesized using techniques and methods known in the art (see, e.g., Edelheit et al. BMC Biotechnol. 2009 Jun. 30:9:61).

Also provided herein are novel PBCV NYs1 ligases, where point mutations are introduced into the parent ligase of PB-1 with the sequence provided in SEQ ID NO:1 or SEQ ID NO:2 to disrupt intermolecular interactions, such as hydrogen bonds and salt bridges, that are thought to contribute to aggregation of the parent ligase. Preliminary structural analyses revealed key intermolecular interactions with distances of less than 4 Å between amino acid residues. Tables 3 and 4 provide pairs of amino acid residues that participate in hydrogen bonds and salt bridges, respectively. The pairs of amino acid residues shown in Tables 3 and 4 were identified to participate in the aforementioned intermolecular interactions with distances of less than 4 Å, and mutations to one or both residues in Tables 3 and 4 are considered as strategies for developing variants of the parent ligase (e.g., PB-1) with resistance to aggregation.

TABLE 3
Pairs of amino acid residues identified from structural analyses that form
hydrogen bonds with distances of less than 4 Å.

| | |
|---|---|
| Arg153 | Thr201 |
| Arg176 | Asp297 |
| Arg285 | Glu237 |
| Arg32 | Glu296 |
| Arg42 | Asp297 |
| Arg48 | Ser265 |
| Arg48 | Asp258 |
| Asn118 | Met88 |
| Asn224 | Thr53 |
| Asn224 | Phe54 |
| Asp258 | Gly85 |
| Cys283 | Cys283 |
| Glu181 | Gly242 |
| Glu271 | Lys190 |
| Glu295 | Thr43 |
| His86 | Asp254 |
| Ile257 | Ser256 |
| Lys15 | Tyr227 |
| Lys190 | Trp270 |
| Ser245 | Glu247 |
| Ser256 | Ser245 |
| Thr121 | Lys87 |
| Thr201 | Gln150 |
| Thr259 | Pro294 |
| Thr43 | Glu295 |

TABLE 4
Pairs of amino acid residues identified from structural analyses that form salt
bridges with distances of less than 4 Å.

| | |
|---|---|
| Arg176 | Asp297 |
| Arg285 | Glu237 |
| Arg42 | Asp297 |
| Arg48 | Asp258 |
| His86 | Asp254 |
| Lys171 | Asp241 |

A computational mutation analysis was performed with aggregation propensity software to evaluate the open and closed conformations (i.e., DNA bound) of the parent ligase, PB-1, having to identify amino acid residues in aggregation prone regions. Using static structures of the two open conformations and one closed conformation of PB-1 were used as inputs into two aggregation propensity software (e.g., AGGRESCAN and SolubiS), where the sequence of PB-1 used for the aggregation propensity software includes an N-terminus His-tag as provided in SEQ ID NO:19 (also referred herein as PB-1_HT). Simulated structures/models provide snapshots of a protein in a dynamic states, which facilitates accurate determination if an aggregation prone region is exposed to the solvent, and thus, simulated models of the open and closed conformations with rotation of the C-terminal domain and protein flexibility were also used in aggregation propensity software.

After 12 runs with two aggregation propensity software using two open conformations and one closed conformation of PB-1_HT. Using the closed conformation of PB-1_HT, a total of 278 mutations were identified with two aggregation propensity software, where 58 mutations exhibited improved aggregation scores and stability. Of these 58 mutations, 9 mutations identified in both static and simulated models, which included S104E, V254K, S256K, G258P, G258D, G258E, V253R, S256E, and V253K in SEQ ID NO:19. Using the first open conformation of PB-1_HT, 278 mutations were identified with two aggregation propensity software, where 56 mutations exhibited improved aggregation scores and stability. Of these 56 mutations, 5 mutations identified in both static and simulated models, which included S256K, S256R, S256E, V253K, and V253R in SEQ ID NO:19. Using the second open conformation of PB-1_HT, 278 mutations were identified with two aggregation propensity software, where 56 mutations exhibited improved aggregation scores and stability. Of these 56 mutations, 6 mutations identified in both static and simulated models, which included S104E, S256K, S256R, S256E, G258P, V254K in SEQ ID NO:19. From the total of 834 mutations arising from 278 mutations for each of the three conformations, this quantity of mutations was narrowed to a total of 169 mutations based on the stability of the mutations and the reduced propensity of the mutations to contribute to aggregation, which was further filtered to prioritize the most frequent mutations observed across all 12 runs, which resulted in a total of 21 unique mutations.

Tabulated in Table 5 are mutations selected for further analysis based on the computational mutation analysis with the two aggregation propensity software. While Table 5 provides point mutations corresponding to amino acid positions of SEQ ID NO:19, one having ordinary skill in the art would understand that SEQ ID NO:19 and SEQ ID NO:1 are related, as SEQ ID NO:1 provides the amino acid sequence of wild type PBCV NYs1 ligase (referred herein as PB-1), and SEQ ID NO:19 provides the amino acid sequence of wild type PBCV NYs1 ligase including a His-tag and linker at the N-terminus of the ligase (referred herein as PB-1_HT). Any amino acid position after amino acid position 1 in SEQ ID NO:1 is shifted by 10 amino acid position to identify the corresponding amino acid position in SEQ ID NO:19. For example, amino acid position 244 in SEQ ID NO:1 corresponds to amino acid position 254 in SEQ ID NO:19. Structural analysis of the resides provided in Table 5 revealed solvent exposed residues that do not interact with DNA or the active site (e.g., 1208, 1300, S104, V150, V253, and V254 in SEQ ID NO:19) and residues that interact with DNA (e.g., G258 and S256). Additionally, phenylalanine at amino acid position 255 in SEQ ID NO:19 was found to potentially stabilize the C-terminus of the ligase.

TABLE 5

| Ligase variants of PB-1_HT. These ligase variants harbor point | |
|---|---|
| mutations at amino acid positions corresponding to amino | |
| acid positions provided in SEQ ID NO: 19. Verification of | |
| point mutations in the ligase variants provided infra was | |
| performed by sequencing of the clones of the variants. | |
| Ref # | Point Mutation |
| PB-10 | V254K |
| PB-11 | V254R |
| PB-12 | V254E |
| PB-13 | V254D |
| PB-14 | S104E |
| PB-15 | G258E |
| PB-16 | G258P |
| PB-17 | G258D |
| PB-18 | V253R |
| PB-19 | V253K |
| PB-20 | V253E |
| PB-21 | S256E |
| PB-22 | S256K |
| PB-23 | S256R |
| PB-24 | S256P |
| PB-25 | F255R |
| PB-26 | F255K |
| PB-27 | I300R |
| PB-28 | I300E |
| PB-29 | I300K |
| PB-30 | V150K |
| PB-31 | V150R |
| PB-32 | I208K |
| PB-33 | L225D |

Strategies for the development of ligase variants of PB-9 (SEQ ID NO:22) were contemplated. Tabulated in Table 6 are ligase variants harboring point mutations corresponding to amino acid positions of SEQ ID NO:22. One having ordinary skill in the art would understand that SEQ ID NO:22 and SEQ ID NO:19 are related as SEQ ID NO:19 provides the amino acid sequence of wild type PBCV NYs1 ligase covalently attached to a His-tag and a linker at the N-terminus (referred herein as PB-1_HT), and SEQ ID NO:22 provides the amino acid sequence of wild type PBCV NYs1 ligase covalently attached to a His-tag, flexible linker, amino acid sequence of a Sso7d polypeptide, and linker at the N-terminus of the ligase (referred herein as PB-9). Any amino acid position after amino acid position 1 in SEQ ID NO:19 is shifted by 69 amino acid position to identify the corresponding amino acid position in SEQ ID NO:22. For example, amino acid position 254 in SEQ ID NO:19 corresponds to amino acid position 323 in SEQ ID NO:22. By way of an additional example, ligase variant PB-35 tabulated in Table 6 harbors a V323K point mutation at amino acid position 323 in SEQ ID NO:22 (PB-9), which corresponds to amino acid position 254 in SEQ ID NO:19 (PB-1_HT) and corresponds to amino acid position 244 in SEQ ID NO:1 (PB-1).

TABLE 6
Ligase variants of PB-9. These ligase variants harbor point
mutations at amino acid positions corresponding to amino acid positions provided in SEQ ID
NO: 22 (PB-9), which are functionally equivalent to amino acid positions provided in SEQ ID
NO: 1 (PB-1). Verification of point mutations in the ligase variants provided infra was performed
by sequencing of the clones of the variants.

| | Point Mutation in | Point Mutation in |
|---|---|---|
| Ref # | SEQ ID NO: 1 | SEQ ID NO: 22 |
| PB-35 | V244K | V323K |
| PB-36 | V244R | V323R |
| PB-37 | V244E | V323E |
| PB-38 | V244D | V323D |
| PB-39 | S94E | S173E |
| PB-40 | G248E | G327E |
| PB-41 | G248P | G327P |
| PB-42 | G248D | G327D |
| PB-43 | V243R | V322R |
| PB-44 | V243K | V322K |
| PB-45 | V243E | V322E |
| PB-46 | S246E | S325E |
| PB-47 | S246K | S325K |
| PB-48 | S246R | S325R |
| PB-49 | S246P | S325P |
| PB-50 | F245R | F324R |
| PB-51 | F245K | F324K |
| PB-52 | I290R | I369R |
| PB-53 | I290E | I369E |
| PB-54 | I290K | I369K |
| PB-55 | V140K | V219K |
| PB-56 | V140R | V219R |
| PB-57 | I198K | I277K |
| PB-58 | L215D | L294D |

Following the evaluation of ligase variants of PB-9 harboring the point mutations described in Table 6 for ligase activity, thermostability, and resistance to aggregation, five point mutations were identified to increase solubility while maintaining ligation activity. These point mutations included V323K in SEQ ID NO:22, which is functionally equivalent to V244K in SEQ ID NO:1; V323R in SEQ ID NO:22, which is functionally equivalent to V244R in SEQ ID NO:1; S325E in SEQ ID NO:22, which is functionally equivalent to S246E in SEQ ID NO:1; V219K in SEQ ID NO:22, which is functionally equivalent to V140K in SEQ ID NO:1; and V322K in SEQ ID NO:22, which is functionally equivalent to V243K in SEQ ID NO:1. These five mutations were used for the development of ligase variants of PB-9 with two point mutations as described in Table 7.

Table 7
Ligase variants of PB-9 harboring two point mutations. These
ligase variants harbor point mutations at amino acid positions corresponding to amino acid
positions provided in SEQ ID NO: 22 (PB-9), which are functionally equivalent to amino acid
positions provided in SEQ ID NO: 1 (PB-1). Verification of point mutations in the ligase variants
provided infra was performed by sequencing of the clones of the variants.

| | | | Double Point | Double Point |
|---|---|---|---|---|
| | | | Mutations in | Mutations in |
| Ref # | Template Parent | Mutation Parent | SEQ ID NO: 1 | SEQ ID NO: 22 |
| PB-59 | PB-35 | PB-46 | V244K and S246E | V323K and S325E |
| PB-60 | PB-35 | PB-55 | V244K and V140K | V323K and V219K |
| PB-61 | PB-35 | PB-44 | V244K and V243K | V323K and V322K |
| PB-62 | PB-36 | PB-46 | V244R and S246E | V323R and S325E |
| PB-63 | PB-36 | PB-55 | V244R and V140K | V323R and V219K |
| PB-64 | PB-36 | PB-44 | V244R and V243K | V323R and V322K |
| PB-65 | PB-46 | PB-55 | S246E and V140K | S325E and V219K |
| PB-66 | PB-46 | PB-44 | S246E and V243K | S325E and V322K |
| PB-67 | PB-55 | PB-44 | V140K and V243K | V219K and V322K |

In addition to introducing single point mutations to the ligases described herein for the development to robust ligases, PEGylation was explored as an avenue to improve the solubility and stability of the ligases. A study was conducted to covalently attach PEG₄ and PEG₁₂ onto PB-9 (SEQ ID NO:22) by incubating PB-9 in the presence of commercially available MS(PEG)₄ and MS(PEG)₁₂ at 5×, 10×, or 15× molar ratio of the pegylation reagent to PB-9. FIG. 7 shows bands corresponding to PB-9 following incubation with MS(PEG)₄ and MS(PEG)₁₂ at 5×, 10×, or 15× molar ratio. Lane 1 shows the molecular weight ladder. Lanes 2 and 9 show the band of purified PB-9 incubated in buffer as a control. Lanes 3, 4, and 5 show bands of PB-9 incubated with MS(PEG)₄ at 5×, 10×, or 15× molar ratios, respectively. Lanes 6, 7, and 8 show bands of PB-9 incubated with MS(PEG)₁₂ at 5×, 10×, or 15× molar ratios, respectively. Concentration dependent increase in the molecular weight of PB-9 (e.g., an upward shift in band migration) with the higher molar ratios of (PEG)₄ and (PEG)₁₂ suggested that all ligases were modified in the sample. We observed improved solubility and thermostability using higher molecular weight pegylation reagents at lower molar ratios. In particular, PEGylating PB-9 with (PEG)₁₂ at a molar ratio of 5× increased ligase activity and thermostability.

An additional study was performed using a higher molecular weight pegylation reagent, (PEG)₂₄ to install PEG moieties onto PB-1 (SEQ ID NO:1), PB-1_HT (SEQ ID NO:19) and PB-9 (SEQ ID NO:22) as described supra. FIG. 8 shows bands corresponding to PB-1_HT and PB-9 following incubation with MS(PEG)₂₄ at 0×, 1×, 2×, and 3× molar ratio. Lane 1 shows the molecular weight ladder. Lanes 2 and 9 show the bands of PB-1_HT and PB-9 incubated in buffer as a control. Lanes 3, 4, and 5 show bands of PB-1_HT incubated with MS(PEG)₂₄ at 1×, 2×, or 3× molar ratios, respectively. Lanes 6, 7, and 8 show bands of PB-9 incubated with MS(PEG)₂₄ at 1×, 2×, or 3× molar ratios, respectively. Following PEGylation of ligase variant PB-9, the ligation activity was evaluated in the ligation activity assay as described infra.

Additional studies were performed appending polyethylene glycol moieties to the ligase variants. N-Hydroxysuccinimide (NHS) esters are the most popular type of reactive group used for protein modification. In pH 7-9 buffers, NHS-ester reagents react efficiently with primary amino groups (—NH2) by nucleophilic attack, forming amide bonds and releasing the NHS. Proteins typically have many sites for labeling, including the primary amine in the side chain of each lysine (K) residue and the N-terminus of each polypeptide. The MS(PEG)n reagents are readily soluble in water or organic solvents such as DMSO, methylene chloride and DMF.

PEG reagents, MS(PEG)4, MS(PEG)12 and MS(PEG)24 were dissolved in DMSO to have a 250 mM stock solution. Wild type variants were dialyzed using Slide-A-Lyzer MINI Dialysis Device (Thermo, cat #69576) in 1 L PBS+5% glycerol for 2 h. The concentration of the enzyme was measured after dialysis and 2×, 5× and 10× molar excess of MS(PEG)4, MS(PEG)12 or MS(PEG)24 were added to the enzyme in 50 ml-Falcon tube and incubated on ice while stirring using a stir bar (VWR cat #58949-272). When the PEGylation reaction is completed the enzyme was dialyzed using Slide-A-Lyzer MINI Dialysis Device (Thermo, cat #69576) in 1 L enzyme storage buffer for 2 h to remove the NHS leaving group, excess of PEG reagent, and PBS. PEGylated PB-1 demonstrated significantly increased solubility, and ˜30% ligation activity compared to non-PEGylated PB-1.16 uM of PB-1 was incubated in direct ligation buffer at 37° C. C for 1 hour, then 42° C. for another hour. Absorbance at 400 nM was monitored to reveal formation of aggregates. Samples at 1 hour and 2 hours were taken, centrifuged, and the supernatant was loaded on an SDS PAGE to confirm presence of soluble ligase. Data confirms that all PEGylated PB-1 samples have significantly delayed aggregation. The SDS PAGE of each sample confirms that each PEGylated ligase has significant protein remaining in the soluble fraction after 2 hours, with 4× and 5×PEG24 samples retaining nearly all protein in the soluble fraction.

Following the production of ligase variants described herein, ligation activities of the variants were scrutinized in a real time ligation activity assay adapted from Tang et al. (Nucleic Acids Res. 2005; 33(11): e97). This ligation activity assay uses molecular beacons to facilitate real time monitoring of ligation of a nicked polynucleotide substrate, where the nicked polynucleotide substrate is hybridized on the molecular beacon, and the kinetics thereof. FIG. 2 illustrates the components of the ligation assay used to measure the ligation ability of the ligases described herein. The molecular beacon, left, includes a loop region, where a portion of the loop is capable of specifically hybridizing with the complementary sequence of Oligo A and the remainder of the loop is capable of specifically hybridizing with the complementary sequence of Oligo B; a fluorophore at the 5′ end of the molecular beacon (depicted as a circle); and a quencher moiety at the 3′ end of the molecular beacon (depicted as a square). A ligatable nick forms as a result of the hybridization of Oligo A and Oligo B to the molecular beacon. In the presence of a ligase (e.g., a ligase variant described herein), the nick between Oligo A and Oligo B is sealed, which results in the opening the molecular beacon, release of the fluorophore from the quencher moiety, and fluorescence of the fluorophore (depicted by the haze behind the fluorophore). Measurement of fluorescence is indicative of the ligation of the nick site by the ligase described herein as fluorescence is quenched when the fluorophore is in close proximity to the quencher moiety prior to the ligation of the nick site. Protocols used for the ligation activity assay and measurement of ligation activity are described infra.

Hybridization of Oligo A and Oligo B to molecular beacon and ligation: To hybridize Oligo A and Oligo B to the molecular beacon, 0.6 μM of Oligo A and 0.6 μM of Oligo B incubated with 0.3 μM of DNA or RNA molecular beacon in a hybridization buffer (50 mM Tris pH 8, 75 mM LiCl, 3 mM MgCl₂, 0.025% Triton™ X-100, 0.1 mM ATP, and 10 mM DTT) at 37° C. In embodiments, the hybridization buffer includes reagents to reduce non-specific binding, such as bovine serum albumin (e.g., 15 μg/mL BSA). Following 10 minutes of incubation, 38 μL of the hybridization reaction were added to a qPCR plate, and to the qPCR plate incubated on ice was added 0.6 μM of a ligase (e.g., a ligase described herein) prior to performing a qPCR protocol for real time monitoring of ligation. In embodiments, Oligo A and Oligo B independently includes deoxyribonucleotides. In embodiments, Oligo A and Oligo B independently includes ribonucleotides. In embodiments, the molecular beacon described herein is a DNA molecular beacon. In embodiments, the molecular beacon described herein is a RNA molecular beacon.

Real time monitoring of ligation with qPCR: The reaction incubated at 4° C. for 1 minute. The reaction temperature was increased to 37° C. and incubated for 1 second prior to the measurement of fluorescence. Following the measurement of the 1 second time point, the reaction was incubated at 37° C. for 1 minute prior to the measurement of fluorescence. Measurement of fluorescence was repeated for an additional 29 1-minute time points following the incubation at 42° C. for 1 minute for the remainder of the 30-minute incubation period. In addition, the ligation activity of the ligase described herein was also monitored at 42° C. To monitor the ligation activity at 42° C., the reaction incubated at 4° C. for 1 minute. The reaction temperature was increased to 42° C. and incubated for 1 second prior to the measurement of fluorescence. Following the measurement of the 1 second time point, the reaction was incubated at 42° C. for 1 minute prior to the measurement of fluorescence. Measurement of fluorescence was repeated for an additional 29 1-minute time points following the incubation at 42° C. for 1 minute for the remainder of the 30-minute incubation period.

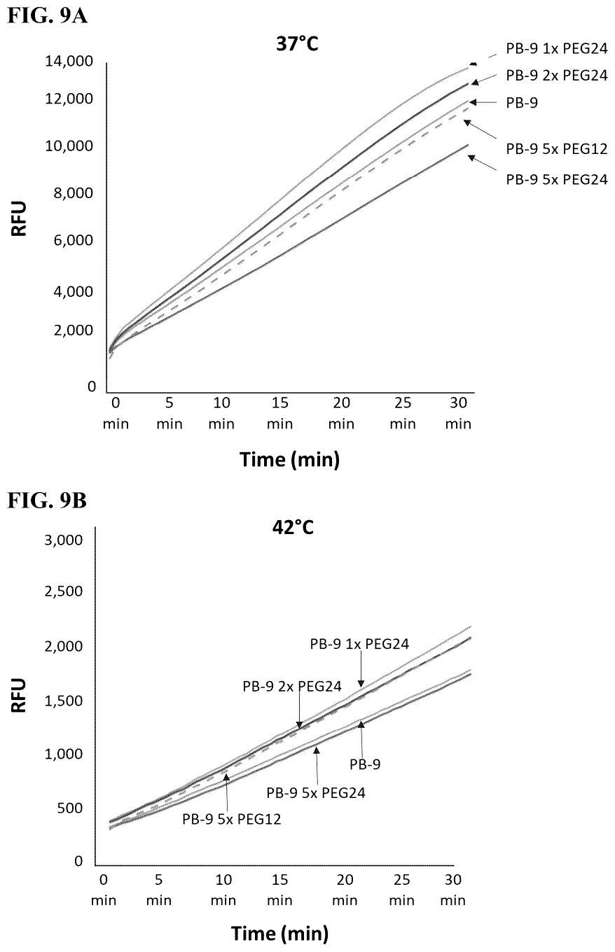

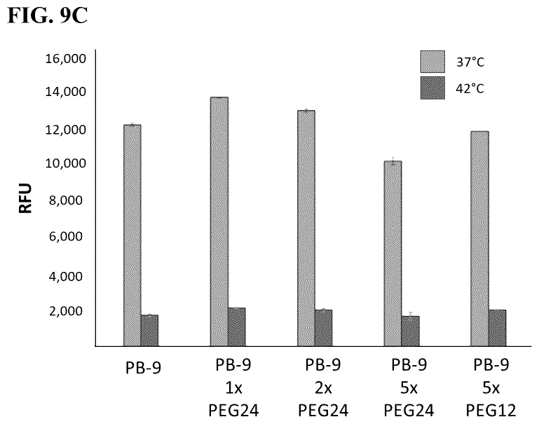

A study was performed to evaluate ligation activity of pegylated ligase variant PB-9 at 37° C. and 42° C. for ligase variants PB-9 incubated with 1× (PEG)₂₄, 5× (PEG)₂₄, 2× (PEG)₂₄, and 5× (PEG)₁₂. The procedure for the ligation activity assay is as described supra. Briefly, 10 nM of each pegylated ligase variant of PB-9 was incubated with 600 nM DNA Oligo A and Oligo B hybridized onto a DNA-DNA molecular beacon in hybridization buffer at 37° C. and 42° C. FIGS. 9A and 9B depict ligation reaction rates as fluorescence measured during the ligation reaction with pegylated ligase variants of PB-9 at 37° C. and 42° C. as a function of time, respectively. FIG. 9C provides the fluorescence measured for ligase variant PB-9 incubated with 1× (PEG)₂₄, 2× (PEG)₂₄, 5× (PEG)₂₄, and 5× (PEG)₁₂ following the incubation with DNA molecular beacon at 37° C. (shown on the left in lighter shade) and 42° C. (shown on the right in darker shade). At 37° C., PB-9 incubated with 1× (PEG)₂₄ and 2× (PEG)₂₄ displayed slightly higher reaction rates compared to non-pegylated PB-9, PB-9 incubated with 5× (PEG)₂₄, PB-9 incubated with 5× (PEG)₁₂. At 42° C., PB-9 incubated with 1× (PEG)₂₄ and 2× (PEG)₂₄ displayed slightly higher reaction rates compared to non-pegylated PB-9, PB-9 incubated with 5× (PEG)₂₄, PB-9 incubated with 5× (PEG)₁₂. After demonstrating enhanced ligation rates afforded from PEGylating ligase variant PB-9, these pegylated ligase variants of PB-9 were evaluated for stability and solubility in an aggregation assay using enzyme concentrations up to 16 μM. PB-9 includes a polyhistidine tag (His-tag), which was appended to the wild-type ligase (PB-1) to facilitate purification and characterization. Experimental results demonstrated favorable performance metrics for the His₉-tagged variant, including but not limited to enhanced expression, purification yield, and enzymatic activity. However, it is understood that the observed effects are not dependent on the presence of the His-tag and similarly apply to the corresponding non-tagged ligase variant, PB-1.

### Example 3. Quantifying Thermostability

Described herein are methods of ligating polynucleotide sequences using a thermostable ligase. The thermostability of the ligase described herein was compared to PBCV-1 DNA ligase (commercially available as SplintR®; SplintR® is a trademark of New England Biolabs). Thermostability studies were performed using ligation buffer (50 mM Tris pH 8, 75 mM LiCl, 3 mM MgCl₂, 0.025% Triton™ X-100, 0.1 mM ATP, and 10 mM DTT), 16 μM of PB-1, and 16 μM of PBCV-1 DNA ligase. Each ligase was incubated in ligation buffer for 20 minutes at room temperature, 30° C., 37° C., and 45° C. at a final concentration of 16 μM. Following 20 minutes incubation, each reaction vessel containing a ligase were centrifuged and the supernatant was retained. The concentration of the ligase in each supernatant was quantified prior to loading onto a 4-12% Bis-Tris gel to run at 200 V for 25 minutes. As a control, 4.4 μg of PBCV-1 DNA ligase was also added to the gel. Differences in thermostability between the ligase described herein and PBCV-1 DNA ligase was ascertained using measurements corresponding to the soluble fraction of the ligase in the supernatant.

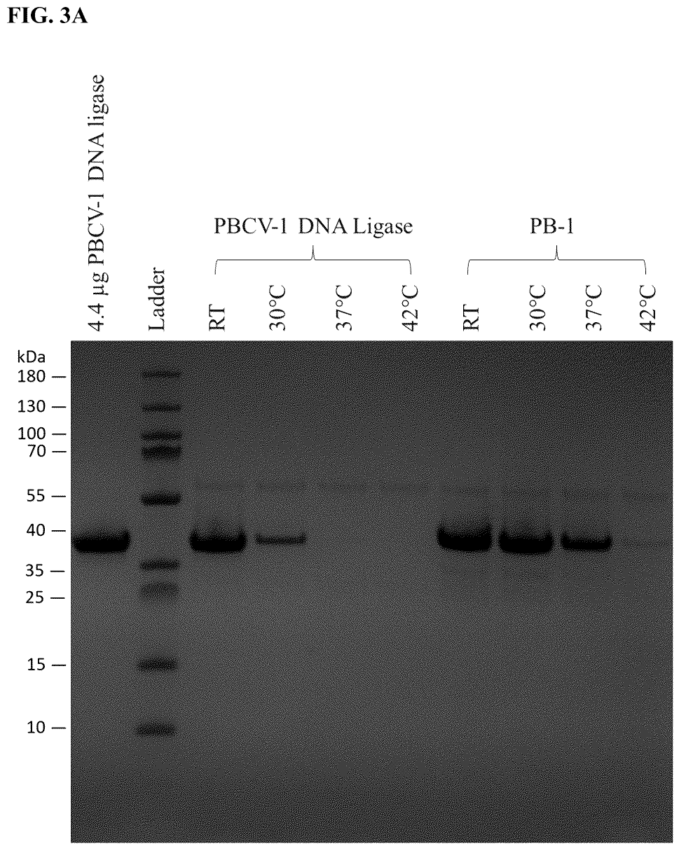

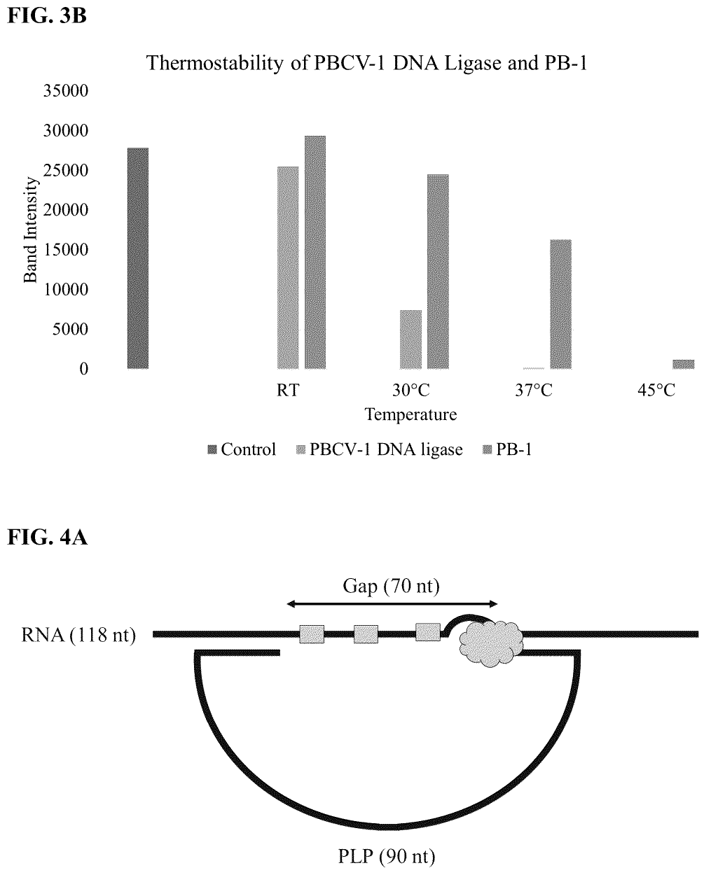

FIG. 3A provides the gel with bands corresponding to supernatants from PBCV-1 DNA ligase (lanes 3-6) and PB-1 (lanes 7-10) following the evaluation of the thermostability of each ligase at room temperature, 30° C., 37° C., and 45° C. FIG. 3B provides the quantification of the band intensity of the bands shown in lanes 3-10 of FIG. 3A. The improved thermostability of PB-1 relative to PBCV-1 DNA ligase was underscored by the presence of stronger bands at 30° C., 37° C., and 45° C. for PB-1 relative to PBCV-1 DNA ligase at the indicated temperatures (FIG. 3A). As shown in FIG. 3B, the quantity of the thermostable fraction of PB-1 was 3-fold and 108-fold higher than the thermostable fraction of PBCV-1 DNA ligase at 30° C. and 37° C., respectively, wherein fold differences were calculated from the quotient of the band intensity of PB-1 at an indicated temperature (dividend) and the band intensity of PBCV-1 DNA ligase at the indicated temperature (divisor). Interestingly, there was a small quantity of the soluble fraction PB-1 observed at 45° C., whereas PCBV-1 DNA ligase precipitated from the supernatant at this temperature (FIGS. 3A and 3B). In a separate study, Applicant determined that PBCV-1 DNA ligase aggregated extensively and lost more than 75% of its activity at 37° C. Higher thermostability of PB-1 relative to PBCV-1 DNA ligase enables improved utility of PB-1 in workflows requiring higher temperatures (e.g., up to 45° C. as shown in FIG. 3B), resistance to aggregation, and improved yields following purification due to its inherent ability to remain soluble during the purification process.

### Example 4. In Vitro Ligation Using Thermostable Ligases

Many workflows used for in situ spatial sequencing rely on padlock probes to selectively hybridize with complementary polynucleotide sequences on RNA and cDNA directly in a cell or tissue. To facilitate detection of a target sequence using a targeted padlock probe, the padlock probe must first hybridize with a complementary sequence, after which probe is ligated to generate a circular polynucleotide, which is subsequently subjected to amplification and detection (see, e.g., U.S. Pat. No. 11,492,662). Alternatively, the targeted padlock probe could hybridize to sequences that flank the target sequences on a nucleic acid molecule, which results in a gap between the sequences hybridized to the padlock probe. To fill the gap, an RNA-dependent polymerase is used to fill the gap in a template-dependent manner, which generates a nicked substrate recognized and sealed by a ligase (see, e.g., U.S. Pat. No. 11,434,525). Following the gap filling and ligation reaction, the ligated product is subjected to amplification to facilitate the detection of the target sequence. In embodiments, the targeted padlock probe may consist of DNA and is capable of hybridizing with sequences flanking a target sequence on a mRNA transcript. In embodiments, the targeted padlock probe consisting of DNA hybridizes to a complementary RNA sequence, and the ligation of the DNA padlock probe occurs with PB-1 or a variant thereof.

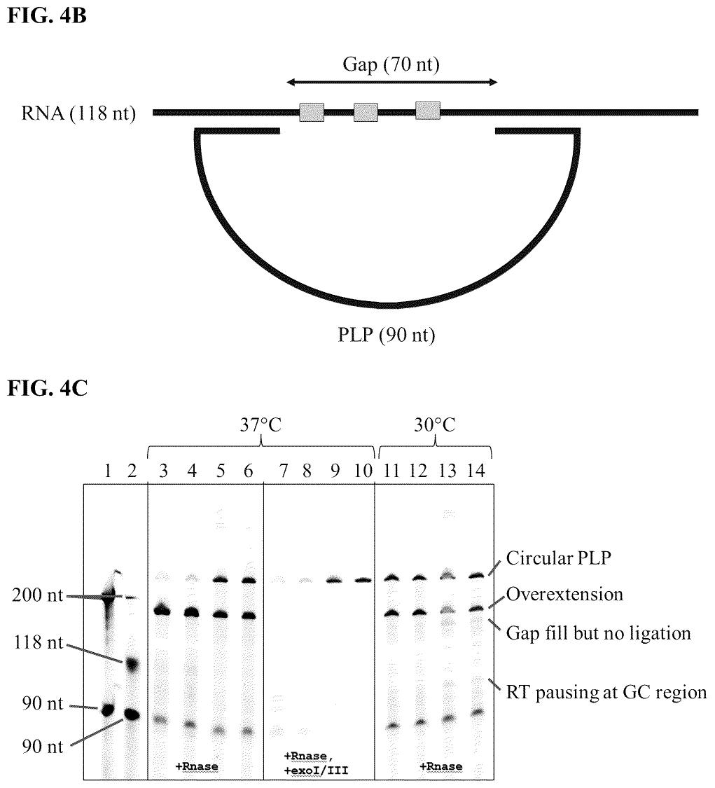

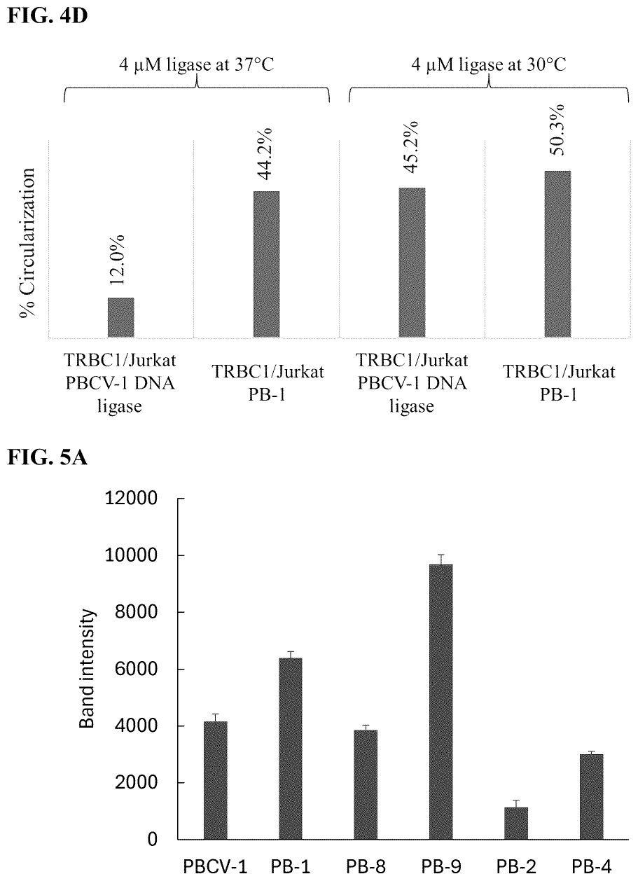

While gap filling prior to ligation enables capturing a sequence of the target polynucleotide in the ligated padlock probe, key challenges of this approach limit its efficiency and utility. For example, experimental conditions for gap filling and ligation require for the gap filling polymerase and the ligase to be present in the same reaction vessel simultaneously for ligation to promptly occur once the gap filling reaction completes. The precise balance of gap filling and ligation is highly critical as overextension of the gap sequence and displacement of the 5′ foot of the padlock probe by the polymerase has been reported and shown to impede the ligation of the padlock probe (see, e.g., Chen et al. Nucleic Acids Res. 2018 Feb. 28; 46(4):e22). To ensure effective ligation following gap filling, the ligase must be present at a sufficiently high concentration to promote the displacement of the polymerase from the nicked substrate to favor ligation. However, high concentrations of ligases (e.g., up to 16 μM of SplintR®) was shown to cause aggregation, which reduces the effective concentration of the ligases available for the ligation reaction (data not shown). Another key consideration is the requirement for both polymerase and ligase to be stable and tolerant to the experimental conditions. For example, workflows featuring gap filling with Moloney Murine Leukemia Virus (M-MLV) reverse transcriptase, require reaction temperatures to be 37° C. (and up to 42° C. for M-MLV H-minus reverse transcriptase) as lower temperatures promote the formation of secondary structures in RNA that impedes the gap filling activity of M-MLV reverse transcriptase (see, e.g., Arezi et al. Nucleic Acids Res. 2009 February; 37(2): 473-481). FIG. 4A depicts the presence of the secondary structure in mRNA (depicted as a bump in the mRNA transcript near the 3′ foot of the padlock probe) with GC-rich regions (depicted by the rectangle shapes within the gap region of the mRNA transcript). This secondary structure causes the reverse transcriptase (depicted as a cloud) to progress slowly through this region (see, e.g., Price et al. PLoS One. 2017; 12(2): e0173023). However, the utility of M-MLV reverse transcriptase for gap filling in combination with PBCV-1 DNA ligase to ligate a DNA padlock probe hybridized to an RNA molecule would be hindered by the aggregation of PBCV-1 DNA ligase at temperatures higher than 30° C. as shown in FIG. 3B (see Example 2). As such, a robust and thermotolerant ligase capable of ligating DNA templates splinted by a complementary RNA template is greatly needed to facilitate efficient detection of polynucleotides in situ.

Described herein are methods and compositions directed towards improving ligation DNA templates hybridized to a complementary RNA template using a thermostable ligase. An in vitro study was performed to evaluate the ability of PB-1 and PBCV-1 DNA ligase to ligate a DNA padlock probe following gap filling by M-MLV reverse transcriptase in Jurkat cell line. A DNA padlock probe was designed to hybridize to a mRNA transcript encoding the T-cell receptor β chain constant domain, TCRβ-1. A schematic of the DNA padlock probe (90 nucleotides in length) and the mRNA transcript targeted by the DNA padlock probe are shown in FIG. 4B. The gap between the 5′ foot and the 3′ foot of the PLP is 70 nucleotides in length, which may include GC-rich regions. Briefly, the protocols for gap filling and ligation are as follows.

Hybridization, Gap Fill, and Probe Ligation: All gap fill and ligation reactions included 0.5 μM RNA:DNA hybrid, 80 nM M-MLV reverse transcriptase H(−), 100 μM dNTPs, and PB-1 or PBCV-1 DNA ligase (New England Biolabs Catalog #M0375S) in hybridization buffer. Each gap fill and ligation incubated for 2 hours at 30° C. or 37° C. to fill the gap and ligate the DNA PLP to generate a circular PLP. Experimental concentrations of PB-1 or PBCV-1 DNA ligase included 4 μM or 10 μM. The comparative ability of PB-1 or PBCV-1 DNA ligase to ligate the targeted DNA PLP including the 70 nucleotide gap were evaluated using gel electrophoresis.

FIG. 4C provides bands corresponding to products formed during the gap fill and ligation reactions described supra. Controls loaded into lane 1 of the gel included single stranded DNA templates of 90 and 200 nucleotides. Controls loaded into lane 2 of the gel included the DNA PLP of 90 nucleotides, the RNA template of 118 nucleotides, and a linear polynucleotide marker of 200 nucleotides; this control represented an unsuccessful gap fill and ligation reaction. Lanes 3-10 corresponded to products formed during gap fill and ligation at 37° C., where lanes 3 and 4 corresponded to products formed in the presence of M-MLV reverse transcriptase H(−) and 10 μM and 4 μM of PBCV-1 DNA ligase, respectively. Lanes 5 and 6 corresponded to products formed in the presence of M-MLV reverse transcriptase H(−) and 10 μM and 4 μM of PB-1, respectively. Lanes 3-6 show bands in the presence of RNase, while lanes 7-10 correspond to bands in the presence of RNase, exonuclease I, and exonuclease III. The presence of RNase, exonuclease I, and/or exonuclease III degrades RNA, linear ssDNA, and linear dsDNA, respectively. Lanes 11-14 corresponded to products formed during gap fill and ligation at 30° C., where lanes 11 and 12 corresponded to products formed in the presence of M-MLV reverse transcriptase H(−) and 10 μM and 4 μM of PBCV-1 DNA ligase, respectively. Lanes 13 and 14 corresponded to products formed in the presence of M-MLV reverse transcriptase H(−) and 10 μM and 4 μM of PB-1, respectively. The desired circularized PLP including the 90 nucleotides of the PLP and 70 nucleotides from the gap sequence complementary to the RNA template has a length of 160 nucleotides; the circularized DNA PLP migrates at a slower rate compared to the linear DNA molecule of the same size (see, e.g., Stellwagen et al. J Chromatogr A. 2009 Mar. 6; 1216(10): 1917-1929). Linear products corresponding to an overextension event by 24 nucleotides are also shown and are 184 nucleotides in length (i.e., 90 nt from PLP, 70 nt from RNA template, and 24 nt due to overextension). Linear products corresponding to a complete gap filling but unsuccessful ligation were observed and had 160 nucleotides; these linear DNA products migrate faster than the circular PLP of the same size as described supra. Lastly, linear DNA products formed as a result of MLV reverse transcriptase H(−) pausing at the first GC stretch in the gap was also observed and had 102 nucleotides (i.e., 90 nt from PLP and 12 nt).

FIG. 4D reports the circularization % of the DNA PLP by PB-1 and PBCV-1 DNA ligase, where circularization % was calculated as a percentage of the quotient of the intensity of the circularized band and the sum of band intensities from circularized band and overextension band. Intensities of the bands shown in the gel were calculated using ImageJ, and the circularization % are shown for gap fill and ligation reactions with 4 μM of the ligases from FIG. 4C.

As shown in FIGS. 4C and 4D, PB-1 retains significant activity at 37° C. as 50% of the PLP was circularized by PB-1, whereas only 12% of the PLP was circularized at the same temperature by PBCV-1 DNA ligase. The increased ligation activity of PB-1 relative to PBCV-1 DNA ligase at 37° C. corroborates with its increased thermostability (evidenced by the increased quantity of soluble PB-1 at 37° C. relative to PBCV-1 DNA ligase; see FIGS. 3A and 3B). As such, PB-1 exhibits superior ligation activity compared to PBCV-1 DNA ligase, which is afforded by its increased thermostability, solubility, and resistance to aggregation at higher temperatures.

### Example 5. Direct In Situ Sequencing of Transcripts

We proceeded to perform in situ sequencing using methods described in Example 3 to prepare circular PLPs ligated by PB-1 ligase and PBCV-1 DNA ligase in SupT1 cells. The DNA PLPs used for this study targeted the mRNA transcript encoding the T-cell receptor R chain constant domain, TCRβ-1. The increased efficiency of PB-1 ligase relative to PBCV-1 DNA ligase in generating circular PLPs correlated with brighter clusters and improved sequencing accuracy. For example, in a sequencing run with 50 cycles, there was 29% higher read count for sequences derived from ligation products formed by PB-1 compared to those formed by PBCV-1 DNA ligase (see Table 8). Additionally, the percentage of cells with targets were 15% higher for sequences derived from ligation products formed by PB-1 compared to those formed by PBCV-1 DNA ligase (see Table 8). The increased read count and percentage of cells with targets for sequences derived from ligation products of PB-1 stems from the superior ligation ability of PB-1 compared to PBCV-1 DNA ligase as described in Example 3, which consequently results in greater quantity of circularized polynucleotides available for sequencing. An increased volume of reads from sequences derived from ligation products formed by PB-1 resulted in a sequencing accuracy of 98% compared to a sequencing accuracy of 97% from sequences derived from ligation products formed by PBCV-1 DNA ligase (Table 8).

TABLE 8
Average cycle accuracy, percentage of cells with targets, and
read count for TCR template from SupTI cell line from
sequencing run with 50 cycles using sequences derived
from ligation products formed by PB-1 and PBCV-1 DNA ligase.

| | Avg Cycle | % Cells with | Read Count for |
|---|---|---|---|
| Ligases | Accuracy (%) | Targets | TCR/SupT1 |
| PBCV-1 DNA | 97.35% | 51.90% | 74,579 |
| ligase | 97.01% | 54.82% | 89,720 |
| PB-1 | 98.20% | 61.42% | 110,758 |
| | 98.13% | 61.49% | 101,468 |

### Example 6. Direct In Situ Sequencing of Transcripts

One key influencing factor in the pathophysiological development of a disease stems from the aberrant gene and protein expression of disease-relevant genes and proteins along with the spatial heterogeneity in their abundance and distribution among cells and tissues. Spatial biology techniques, such as in situ sequencing, enables the scrutiny of disease-relevant biomolecules (such as lipids, carbohydrates, nucleic acids, and/or proteins) in the original context of intact tissue, which enables the evaluation of these macromolecules in relation to the tissue architecture and cellular microenvironment, both of which are governed by the intracellular and intercellular communication in situ. Provided herein are methods of using ligase variants described herein in workflows for in situ detection of nucleic acids in tissue sections.

Tissue sections from bone marrow were immobilized onto a solid support (e.g., a glass slide or flow cell) using known techniques in the art. Following immobilization of the tissue on the solid support, the solid support was baked at elevated temperatures (e.g., 30-70° C.) and placed in dark storage at room temperature overnight. The tissue sections were then deparaffinized using xylene followed by 100% EtOH incubation. The slides were dried at 37° C. for 15 mins. Following deparaffinization, the slides were immersed into antigen retrieval buffer (pH 9) and incubated in a pressure cooker. The tissue sections were fixed using 4% paraformaldehyde (PFA) for 20 minutes.

Following the transfer of the tissues onto the solid support, padlock probes targeting the target RNA transcripts were allowed to hybridize with the nucleic acids of interest. In embodiments, 3 unique padlock probes with a sequence capable of hybridizing with a nucleic acid of interest are used to facilitate its detection in situ. In embodiments, 7 unique padlock probes with a sequence capable of hybridizing with a nucleic acid of interest are used to facilitate its detection in situ. In embodiments, 12 unique padlock probes with a sequence capable of hybridizing with a nucleic acid of interest are used to facilitate its detection in situ. Following hybridization, the padlock probes targeting the nucleic acids of interest were ligated using ligases described herein or variants thereof. Following ligation, the ligated padlock probes were amplified using rolling circle amplification. Amplicons corresponding to the padlock probes targeting nucleic acids of interest were sequenced. In embodiments, the nucleic acids of interest were further detected using fluorescent hematoxylin and eosin (H&E) staining.

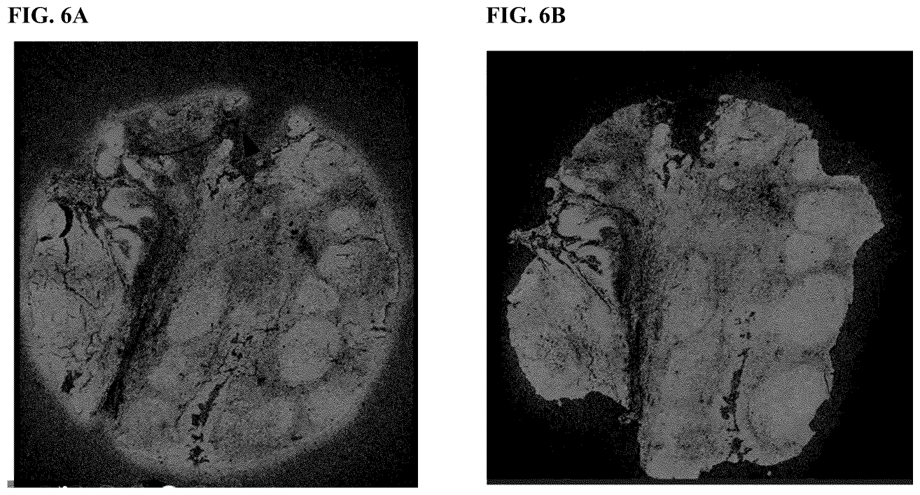

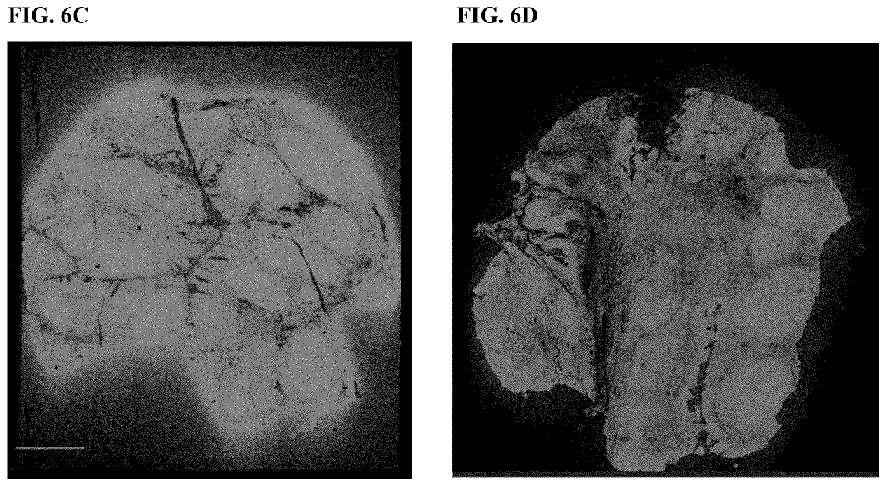

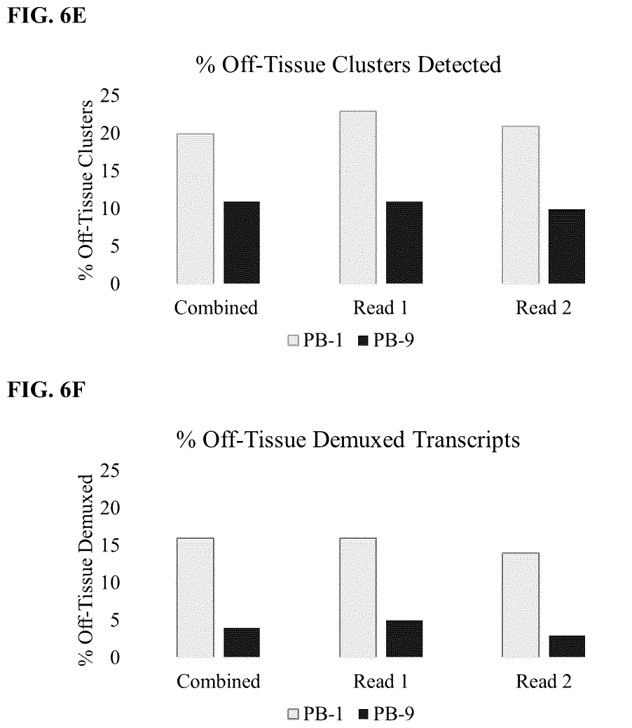

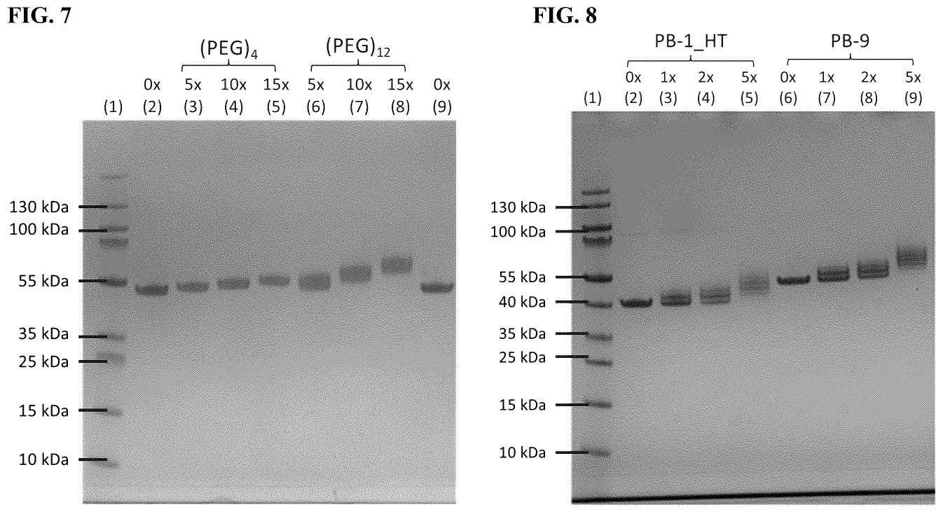

FIGS. 6A-6F provide images of a tissue section from bone marrow attached to a flow cell using methods described supra. Each dot on the image depicts a detected RNA molecule via hybridization of a padlock probe specific to the target RNA molecule, ligation of the padlock probe with a ligase described herein, amplification of the ligated padlock probe via rolling circle amplification and sequencing of the amplified product. FIG. 6A shows the detected transcripts in bone marrow, where ligation of the padlock probe was performed by PB-1 at 30° C. FIG. 6B provides the detection of transcripts in bone marrow, where ligation of the padlock probe was performed by PB-9 at 30° C. FIG. 6C shows the detected transcripts in bone marrow, where ligation of the padlock probe was performed by PB-1 at 35° C. FIG. 6D provides the detection of transcripts in bone marrow, where ligation of the padlock probe was performed by PB-9 at 35° C. Tissue sections shown in FIGS. 6A and 6B were immobilized onto different lanes of the same flow cell. Similarly, tissue sections shown in FIGS. 6C and 6D were immobilized onto different lanes of the same flow cell. While both PB-1 and PB-9 enabled the detection of RNA molecules of interest in bone marrow, the use of PB-9 to ligate target-specific padlock probes resulted in reduced the frequency of off-tissue clusters; this was evidenced by the reduction of detected signal outside the tissue section when PB-9 was used for ligation at 30° C. and 35° C. Quantification of off-tissue clusters and off-tissue demuxed transcripts for sequencing experiments shown in FIGS. 6A and 6B are presented in FIGS. 6E and 6F. Use of PB-9 reduced the percentage of off-tissue clusters on bone marrow by about two-fold compared to the use of PB-1 as shown in FIG. 6E. The percentage of demuxed transcripts reduced by about 4-5-fold when PB-9 was used to ligate the target-specific padlock probes compared to when PB-1 was used.

Alternatively, or additionally, methods of using the flow cell assembly described herein for the detection of proteins of interest without the detection of nucleic acids of interest were also contemplated. For a proteomics workflow, tissues were prepared for transfer to the functionalized tissue glass slide in deparaffinization and heat-induced antigen retrieval steps as described supra and contacted with detection agents targeting proteins of interest. In embodiments, the detection agent is an antibody with an oligonucleotide label, where the determination of the sequence of the oligonucleotide label and its association to the protein of interest is made apriori. In embodiments, the oligonucleotide label is a padlock probe, where the sequence of the padlock probe and its association to the protein of interest is made apriori. Following the binding interaction between target-specific antibody and the protein of interest, padlock probes associated with the proteins of interest were ligated using SplintR® ligase and amplified using rolling circle amplification. Amplicons associated with proteins of interest were sequenced. In embodiments, proteins of interest were further detected using fluorescent hematoxylin and eosin (H&E) staining. In embodiments, about 10, 11, 12, 13, 14, 15, 16, 17, 18, 19, 20, 21, 22, 23, 24, 25, 26, 27, 28, 29, 30, or more proteins of interest are detected. In embodiments, about 10 proteins of interest are detected. In embodiments, about 15 proteins of interest are detected. In embodiments, about 20 proteins of interest are detected. Additionally, methods described herein could be modified to facilitate the combined detection nucleic acids of interest and proteins of interest in the same tissue section.

### Example 7. Preferential Binding to RNA-DNA Over DNA-DNA Templates

Ligases that preferentially ligate RNA-DNA hybrid substrates over DNA-DNA duplexes offer a valuable biochemical feature for RNA-targeted applications. Substrate selectivity of this nature is particularly advantageous for in situ hybridization and ligation workflows, where the objective is to circularize probes specifically bound to RNA targets while minimizing ligation on off target DNA substrates, such as genomic DNA (gDNA). Specificity of this kind is essential in single-cell and spatial transcriptomics, where accurate detection of RNA molecules in complex tissue contexts is often hindered by the pervasive presence of gDNA.

A persistent technical obstacle in these workflows stems from the sequence identity shared between messenger RNA (mRNA) and gDNA. Homology between these nucleic acids results in non-specific hybridization and amplification events, which generate background signals that diminish the dynamic range and precision of gene expression measurements. Signal contamination becomes especially problematic in samples with degraded RNA, including formalin-fixed paraffin-embedded (FFPE) tissues, where distinguishing between RNA- and DNA-derived signals is particularly challenging. To overcome this limitation, a ligation strategy was developed that suppresses gDNA-derived signal by employing ligases with substrate preferences favoring RNA-DNA hybrids. Circularizable probes, such as padlock probes, are introduced to hybridize with both RNA and gDNA targets. However, ligation occurs selectively on RNA-containing hybrids due to the enzymatic specificity of the ligase used.

The general workflow involves detecting target nucleic acids, such as mRNA transcripts, within biological samples. Circularizable oligonucleotide probes are applied to cell or tissue specimens, where they hybridize directly or indirectly to specific target sequences. Each probe contains two terminal regions complementary to adjacent sequences on the target molecule, enabling proximity of the probe ends upon hybridization. Some probe designs include internal features such as molecular barcodes, which support multiplexed identification and quantification. Following hybridization to an RNA target, the probe is ligated into a covalently closed circular molecule through the action of a ligase that preferentially recognizes RNA-DNA hybrid structures. The resulting circular probe can then be amplified or detected using downstream molecular techniques such as rolling circle amplification. The ligase may be a recombinantly produced enzyme or part of a specifically formulated ligase composition exhibiting robust RNA-DNA substrate specificity suitable for in situ workflows. By enabling selective ligation on RNA while avoiding non-specific activity on gDNA, this approach significantly enhances signal specificity, quantitative accuracy, and reproducibility in transcriptomic assays. The method is particularly useful for FFPE and other sample types where RNA degradation and genomic contamination would otherwise compromise analytical fidelity.

Using an Electrophoretic Mobility Shift Assay (EMSA) assay, we explored preference for RNA-DNA templates over DNA-DNA templates. A range of ligase concentrations (0-1000 nM) were incubated in ligation buffer pH 9.0 with either 200 nM nicked dsDNA or 200 nM nicked DNA-RNA splint for 30 minutes at 37° C. Then the reaction was prepared with sample loading buffer and run on a 6% DNA retardation gel. All the bound and unbound fractions of DNA in the EMSAs were quantified using ImageJ, then the ligase concentration was plotted as a function of fraction bound DNA. A curve was fit to the data using the equation to solve for the dissociation constant (kD). Table 9 summarizes the dissociation constants of each EMSA assay from each curve fit.

TABLE 9
Dissociation constants (kD) were determined for various ligase variants using
electrophoretic mobility shift assays (EMSA). Variants for which the assay did not yield
sufficient signal to calculate a reliable dissociation constant are reported as “N/A”.

| | RNA-DNA template | DNA-DNA template |
|---|---|---|
| Ligase | kD (nM) | kD (nM) |
| PB-1 | 580 | 1386 |
| PEGylated PB-1 | 1480 | N/A |
| PB-75 | 331 | 609 |
| PEGylated PB-75 | 363 | N/A |
| T4 DNA ligase | 396 | 445 |

A general trend for all ligases shows a lower dissociation constant for RNA-DNA substrates compared to DNA-DNA substrates. PEGylation of PB-1 significantly reduces the ability of PB-1 to bind to both substrates. Similarly, PEGylated PB-75 also loses a significant amount of binding with nicked dsDNA compared to non-PEGylated PB-75, however, binding to nicked DNA-RNA substrates shows a minimal change in dissociation constant upon PEGylation. This suggests PEGylated ligases are (e.g., 5×PEG24) promising variants for use within RNA-targeted sequencing applications.

SEQUENCES

PB-1 (PBCV NYs1 ligase) (SEQ ID NO: 1)
MTIAKPLLAATLENLDDVKFPCLVTPKIDGIRSLKQQHMLSRTFKPIRNSVMNKLLSELLPEGADGEICIEDST
FQATTSAVMTGHKVYDEKFSYYWFDYVVDDPLKSYTDRVNDMKKYVDDHPHILEHEQVKIIPLIPVEINNIDEL
SQYERDVLAKGFEGVMIRRPDGKYKFGRSTLKEGILLKMKQFKDAEATIISMSPRLKNTNAKSKDNLGYSKRST
HKSGKVEEETMGSIEVDYDGVVFSIGTGEDDEQRKHFWENKDSYIGKLLKFKYFEMGSKDAPRFPVFIGIRHEE
DC
PB-1_trnc (SEQ ID NO: 2)
MTIAKPLLAATLENLDDVKFPCLVTPKIDGIRSLKQQHMLSRTFKPIRNSVMNKLLSELLPEGADGEICIEDST
FQATTSAVMTGHKVYDEKFSYYWFDYVVDDPLKSYTDRVNDMKKYVDDHPHILEHEQVKIIPLIPVEINNIDEL
SQYERDVLAKGFEGVMIRRPDGKYKFGRSTLKEGILLKMKQFKDAEATIISMSPRLKNTNAKSKDNLGYSKRST
HKSGKVEEETMGSIEVDYDGVVFSIGTGFDDEQRKHFWENKDSYIGKLLKFKYFEMGSKDAPRFPVFIGIRHEE
D
SSo7d (SEQ ID NO: 3)
MATVKFKYKGEEKEVDISKIKKVWRVGKMISFTYDEGGGKTGRGAVSEKDAPKELLQMLEKQKK
Sac7d (SEQ ID NO: 4)
MVKVKFKYKGEEKEVDTSKIKKVWRVGKMVSFTYDDNGKTGRGAVSEKDAPKELLDMLARAEREKK
Sac7e (SEQ ID NO: 5)
MAKVRFKYKGEEKEVDTSKIKKVWRVGKMVSFTYDDNGKTGRGAVSEKDAPKELMDMLARAEKKK
Mse7 (SEQ ID NO: 6)
MATKIKFKYKGQDLEVDISKVKKVWKVGKMVSFTYDDNGKTGRGAVSEKDAPKELLNMIGKK
Mcu7 (SEQ ID NO: 7)
MATKIKFKYKGQDLEVDISKVKKVWKVGKMVSFTYDDNGKTGRGAVSEKDAPKELLSMIGKK
Aho7a (SEQ ID NO: 8)
MTTVKFKYKGEEKEVDISKIKKVWRVGKMISFTYDDNGKTGRGAVSEKDAPKELLEKLEKK
Aho7b (SEQ ID NO: 9)
MATKVKFKYKGEEKEVDISKIKKVWRVGKMISFTYDDNGKTGRGAVSEKDAPKELLDKLEKK
Aho7c (SEQ ID NO: 10)
MATKVKFKYKGEEKEVDISKIKKVWRVGKMISFTYDDNGKTGRGAVSEKDAPKELLEKLK
Sto7 (SEQ ID NO: 11)
MVTVKFKYKGEEKEVDISKIKKVWRVGKMISFTYDDNGKTGRGAVSEKDAPKELLQMLEKSGKK
ssh7b (SEQ ID NO: 12)
MVTVKFKYKGEEKEVDTSKIKKVWRVGKMISFTYDEGGGKTGRGAVSEKDAPKELLQMLEKQKK
Sis7a (SEQ ID NO: 13)
MTTVKFKYKGEEKQVDISKIKKVWRVGKMISFTYDEGGGKTGRGAVSEKDAPKELLQMLEKQKK
Sis7b (SEQ ID NO: 14)
MTTVKFKYKGEEKQVDTSKIKKVWRVGKMISFTYDEGGGKTGRGAVSEKDAPKELLQMLEKQKK
Ssh7a (SEQ ID NO: 15)
MATVKFKYKGEEKQVDISKIKKVWRVGKMISFTYDEGGGKTGRGAVSEKDAPKELLQMLEKQKK
PBCV-1 DNA Ligase (SEQ ID NO: 16)
MAITKPLLAATLENIEDVQFPCLATPKIDGIRSVKQTQMLSRTFKPIRNSVMNRLLTELLPEGSDGEISIEGAT
FQDTTSAVMTGHKMYNAKFSYYWFDYVTDDPLKKYIDRVEDMKNYITVHPHILEHAQVKIIPLIPVEINNITEL
LQYERDVLSKGFEGVMIRKPDGKYKFGRSTLKEGILLKMKQFKDAEATIISMTALFKNTNTKTKDNFGYSKRST
HKSGKVEEDVMGSIEVDYDGVVFSIGTGFDADQRRDFWQNKESYIGKMVKFKYFEMGSKDCPRFPVFIGIRHEE
DR
BH-1 (SEQ ID NO: 17)
MPIIKKPLLATNVVEAASIRFPCFVTPKIDGIRAIRVDESLVSRQFKPIPNRKIRETLQNLLPDGADGEIMIPG
SFRDVTSAVMTSKGTESYSKPFTFYWFDFVQTEKDAEKPYLHRMDDMKHYISKHPHIMEQSQATIIPLYPKKIE
SLEELVTFEQYVLDKGFEGVMIRKGDGFYKMGRSTLNEGILLKLKRFSDSEALLIRVNELFNNNNEKKATETGG
WMRPTKKAGLSSSATMGSFTVRTPQNVEFNIGSGFTNEMRIKFYQEKDKLIGKIVKYKYFEIGVKNLPRFPTFL
GFRDPEDIS
cDNA_PB-1 (SEQ ID NO: 18)
ATGCATCATCATCATCATCACGGCGGTTCAGGCACCATTGCGAAACCTTTGTTAGCGGCGACCTTGGAAAATCT
GGATGATGTCAAATTCCCGTGTCTGGTGACCCCGAAAATCGACGGTATCCGCAGCCTGAAACAACAGCACATGC
TGAGCCGCACCTTCAAGCCGATCCGTAATTCTGTCATGAACAAGCTGCTGAGCGAGTTGCTGCCAGAGGGCGCG
GACGGCGAGATTTGCATTGAAGATTCTACGTTTCAGGCAACCACGAGCGCAGTCATGACGGGTCACAAAGTTTA
CGATGAAAAGTTCAGCTATTACTGGTTCGATTATGTTGTTGACGACCCGCTGAAATCGTACACGGATCGTGTGA
ATGACATGAAGAAATACGTTGACGATCACCCGCATATCCTGGAGCACGAACAAGTGAAGATCATTCCGCTGATC
CCGGTTGAGATCAATAACATTGATGAACTGAGCCAGTATGAGCGTGACGTCCTGGCGAAGGGTTTCGAGGGCGT
GATGATCCGCCGCCCGGACGGTAAGTACAAGTTTGGTCGTAGCACGCTGAAGGAAGGCATTCTGCTGAAGATGA
AACAATTTAAAGACGCCGAGGCCACCATTATCAGCATGAGCCCGCGTCTGAAAAACACCAATGCAAAGTCCAAA
GACAACCTGGGTTATAGCAAGCGCTCCACCCACAAATCCGGTAAAGTCGAAGAAGAGACTATGGGCAGCATCGA
AGTGGACTACGACGGCGTAGTGTTTAGCATTGGTACCGGTTTTGACGATGAGCAGCGTAAGCACTTCTGGGAGA
ACAAAGATAGCTACATCGGTAAGCTCCTGAAGTTTAAGTATTTCGAGATGGGTAGCAAAGATGCTCCGCGTTTC
CCGGTTTTTATTGGTATTCGTCACGAAGAGGATTGCTAATAA
PB-1_HT (SEQ ID NO: 19)
MHHHHHHGGSGTIAKPLLAATLENLDDVKFPCLVTPKIDGIRSLKQQHMLSRTFKPIRNSVMNKLLSELLPEGA
DGEICIEDSTFQATTSAVMTGHKVYDEKFSYYWFDYVVDDPLKSYTDRVNDMKKYVDDHPHILEHEQVKIIPLI
PVEINNIDELSQYERDVLAKGFEGVMIRRPDGKYKFGRSTLKEGILLKMKQFKDAEATIISMSPRLKNTNAKSK
DNLGYSKRSTHKSGKVEEETMGSIEVDYDGVVFSIGTGFDDEQRKHFWENKDSYIGKLLKFKYFEMGSKDAPRF
PVFIGIRHEEDC
PB-2_HT (SEQ ID NO: 20)
MHHHHHHGGSGTIAKPLLAATLENLDDVKFPCLVTPKIDGIRSLKQQHMLSRTFKPIRNSVMNKLLSELLPEGA
DGEICIEDSTFQATTSAVMTGHKVYDEKFSYYWFDYVVDDPLKSYTDRVNDMKKYVDDHPHILEHEQVKIIPLI
PVEINNIDELSQYERDVLAKGFEGVMIRRPDGKYKFGRSTLKEGILLKMKQFKDAEATIISMSPRLKNTNAKSK
DNLGYSKRSTHKSGKVEEETMGSIEVDYDGVVFSIGTGFDDEQRKHFWENKDSYIGKLLKFKYFEMGSKDAPRF
PVFIGIRHEED
Linker: GTGGGG (SEQ ID NO: 21)
PB-9 (SEQ ID NO: 22)
MHHHHHHGGSGATVKFKYKGEEKEVDISKIKKVWRVGKMISFTYDEGGGK
TGRGAVSEKDAPKELLQMLEKQKKGTGGGGTIAKPLLAATLENLDDVKFP
CLVTPKIDGIRSLKQQHMLSRTFKPIRNSVMNKLLSELLPEGADGEICIE
DSTFQATTSAVMTGHKVYDEKFSYYWFDYVVDDPLKSYTDRVNDMKKYVD
DHPHILEHEQVKIIPLIPVEINNIDELSQYERDVLAKGFEGVMIRRPDGK
YKFGRSTLKEGILLKMKQFKDAEATIISMSPRLKNTNAKSKDNLGYSKRS
THKSGKVEEETMGSIEVDYDGVVFSIGTGEDDEQRKHFWENKDSYIGKLL
KFKYFEMGSKDAPRFPVFIGIRHEEDC
PB-34 (SEQ ID NO: 23)
MHHHHHHGGSGNKTQLIDVIAEKADLSKSQAKAALESTLAAITESLKKGDAVQLVGFGTFKVNHRAERTGRNPQ
TGKEIKIAAANVPAFVSGKVLKDSVKGGSGGSTIAKPLLAATLENLDDVKFPCLVTPKIDGIRSLKQQHMLSRT
FKPIRNSVMNKLLSELLPEGADGEICIEDSTFQATTSAVMTGHKVYDEKFSYYWFDYVVDDPLKSYTDRVNDMK
KYVDDHPHILEHEQVKIIPLIPVEINNIDELSQYERDVLAKGFEGVMIRRPDGKYKFGRSTLKEGILLKMKQFK
DAEATIISMSPRLKNTNAKSKDNLGYSKRSTHKSGKVEEETMGSIEVDYDGVVFSIGTGEDDEQRKHFWENKDS
YIGKLLKFKYFEMGSKDAPRFPVFIGIRHEED
AT-1 (SEQ ID NO: 24)
MHHHHHHGGSGNKTQLIDVIAEKADLSKSQAKAALESTLAAITESLKKGDAVQLVGFGTFKVNHRAERTGRNPQ
TGKEIKIAAANVPAFVSGKVLKDSVKGGSGGSAIQKPLLAASLKKLSVDDLTFPVYATPKLDGIRALKIDGTIV
SRTFKPIRNTTISNVLMSLLPDGSDGEILSGKTFQDSTSTVMSADAGIGSGTTFFWFDYVKDDPDKGYLDRIAD
MKKFVDSRPEILKDSRVTIVPLIPKKIDTAEELNVFEQWCLDQGFEGVMVRNAGGKYKFGRSTEKEQILVKIKQ
FEDDEAVVIGVSALQTNVNDKKMNELGDMRRTSHKDGKIDLEMLGALDVEWNGIRFGIGTGFDKDTREDLWKRR
DSIIGKIVKFKYFSQGVKTAPRFPVFLGFRDKNDM
HU-alpha (SEQ ID NO: 25)
MNKTQLIDVIAEKADLSKSQAKAALESTLAAITESLKKGDAVQLVGFGTFKVNHRAERTGRNPQTGKEIKIAAA
NVPAFVSGKVLKDSVK
ATCV1_Z187L (SEQ ID NO: 26)
MAIQKPLLAASLKKLSVDDLTFPVYATPKLDGIRALKIDGTIVSRTFKPIRNTTISNVLMSLLPDGSDGEILSG
KTFQDSTSTVMSADAGIGSGTTFFWFDYVKDDPDKGYLDRIADMKKFVDSRPEILKDSRVTIVPLIPKKIDTAE
ELNVFEQWCLDQGFEGVMVRNAGGKYKFGRSTEKEQILVKIKQFEDDEAVVIGVSALQTNVNDKKMNELGDMRR
TSHKDGKIDLEMLGALDVEWNGIRFGIGTGFDKDTREDLWKRRDSIIGKIVKFKYFSQGVKTAPRFPVFLGFRD
KNDM

What is claimed is:

1. A ligase comprising an amino acid sequence that is at least 85% identical to SEQ ID NO:1 covalently attached to a polyethylene glycol moiety.

2. The ligase of claim 1, wherein the polyethylene glycol moiety comprises 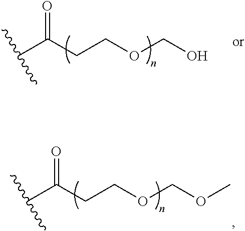
wherein n is 2 to 24.

3. The ligase of claim 2, wherein n is 6, 12, 18, or 24.

4. The ligase of claim 2, wherein n is 24.

5. The ligase of claim 1, comprising a plurality of polyethylene glycol moieties.

6. The ligase of claim 1, wherein the polyethylene glycol moiety is covalently attached to the ligase via a bioconjugate linker.

7. The ligase of claim 1, further comprising one or more substitutions at amino acid position corresponding to position 7, 8, 10, 23, 24, 25, 27, 29, 30, 32, 42, 65, 67, 75, 92, 93, 94, 95, 96, 97, 98, 99, 100, 107, 108, 109, 110, 111, 112, 113, 114, 115, 116, 117, 118, 119, 140, 161, 164, 165, 166, 167, 171, 172, 173, 174, 175, 176, 186, 188, 190, 228, 234, 243, 244, 246, 276, 285, 286, 293, and/or 297 of SEQ ID NO:1.

8. The ligase of claim 1, further comprising a substitution at amino acid position corresponding to position 140, 243, 244, and/or 246.

9. The ligase of claim 8, wherein the substitution at amino acid position corresponding to position 140, 243, 244 is independently an arginine, lysine, histidine, glutamine, asparagine, methionine, or serine.

10. The ligase of claim 8, wherein the substitution at amino acid position corresponding to position 140, 243, 244 is independently a lysine, arginine, or histidine.

11. The ligase of claim 8, wherein the substitution at amino acid position corresponding to position 246 is an aspartic acid, glutamine, asparagine.

12. The ligase of claim 1, comprising an amino acid sequence that is at least 80% identical to a continuous 250 amino acid sequence within SEQ ID NO:1.

13. The ligase of claim 1, further comprising a lysine or arginine at amino acid position corresponding to position 140 of SEQ ID NO:1.

14. The ligase of claim 1, further comprising a histidine tag.

15. The ligase of claim 1, further comprising a lysine, arginine, glutamic acid, aspartic acid, or proline at amino acid position corresponding to position 246 of SEQ ID NO:1.

16. A method for detecting polynucleotide sequences, comprising:
(a) hybridizing a first polynucleotide sequence to a nucleic acid molecule and hybridizing a second polynucleotide sequence to the nucleic acid molecule;
(b) ligating the first polynucleotide sequence and the second polynucleotide sequence together with a ligase to generate a ligated product, wherein the ligase comprises an amino acid sequence that is at least 85% identical to SEQ ID NO:1; and
(c) detecting the ligated product.

17. The method of claim 16, wherein (a)-(c) occur in a cell or tissue.

18. The method of claim 16, further comprising amplifying the ligated product by extending an amplification primer hybridized to the ligated product with a polymerase to generate an extension product.

19. The method of claim 17, further comprising amplifying the ligated product in a cell or tissue.

20. The method of claim 16, wherein detecting comprises sequencing.


SEQUENCE LISTING (26 sequences)

SEQ ID NO: 1 - protein, 298 aa, Paramecium bursaria
MTIAKPLLAATLENLDDVKFPCLVTPKIDGIRSLKQQHMLSRTFKPIRNSVMNKLLSELLPEGADGEICIEDSTFQATTSAVMTGHKVYDEKFSYYWFDYVVDDPLKSYTDRVNDMKKYVDDHPHILEHEQVKIIPLIPVEINNIDELSQYERDVLAKGFEGVMIRRPDGKYKFGRSTLKEGILLKMKQFKDAEATIISMSPRLKNTNAKSKDNLGYSKRSTHKSGKVEEETMGSIEVDYDGVVFSIGTGFDDEQRKHFWENKDSYIGKLLKFKYFEMGSKDAPRFPVFIGIRHEEDC

SEQ ID NO: 2 - protein, 297 aa, Paramecium bursaria
MTIAKPLLAATLENLDDVKFPCLVTPKIDGIRSLKQQHMLSRTFKPIRNSVMNKLLSELLPEGADGEICIEDSTFQATTSAVMTGHKVYDEKFSYYWFDYVVDDPLKSYTDRVNDMKKYVDDHPHILEHEQVKIIPLIPVEINNIDELSQYERDVLAKGFEGVMIRRPDGKYKFGRSTLKEGILLKMKQFKDAEATIISMSPRLKNTNAKSKDNLGYSKRSTHKSGKVEEETMGSIEVDYDGVVFSIGTGFDDEQRKHFWENKDSYIGKLLKFKYFEMGSKDAPRFPVFIGIRHEED

SEQ ID NO: 3 - protein, 64 aa, Sulfolobus solfataricus
MATVKFKYKGEEKEVDISKIKKVWRVGKMISFTYDEGGGKTGRGAVSEKDAPKELLQMLEKQKK

SEQ ID NO: 4 - protein, 66 aa, Sulfolobus acidocaldarius
MVKVKFKYKGEEKEVDTSKIKKVWRVGKMVSFTYDDNGKTGRGAVSEKDAPKELLDMLARAEREKK

SEQ ID NO: 5 - protein, 65 aa, Sulfolobus acidocaldarius
MAKVRFKYKGEEKEVDTSKIKKVWRVGKMVSFTYDDNGKTGRGAVSEKDAPKELMDMLARAEKKK

SEQ ID NO: 6 - protein, 62 aa, Sulfolobus sp.
MATKIKFKYKGQDLEVDISKVKKVWKVGKMVSFTYDDNGKTGRGAVSEKDAPKELLNMIGKK

SEQ ID NO: 7 - protein, 62 aa, Sulfolobus sp.
MATKIKFKYKGQDLEVDISKVKKVWKVGKMVSFTYDDNGKTGRGAVSEKDAPKELLSMIGKK

SEQ ID NO: 8 - protein, 61 aa, unidentified
MTTVKFKYKGEEKEVDISKIKKVWRVGKMISFTYDDNGKTGRGAVSEKDAPKELLEKLEKK

SEQ ID NO: 9 - protein, 62 aa, unidentified
MATKVKFKYKGEEKEVDISKIKKVWRVGKMISFTYDDNGKTGRGAVSEKDAPKELLDKLEKK

SEQ ID NO: 10 - protein, 60 aa, unidentified
MATKVKFKYKGEEKEVDISKIKKVWRVGKMISFTYDDNGKTGRGAVSEKDAPKELLEKLK

SEQ ID NO: 11 - protein, 64 aa, Sulfolobus tokodaii
MVTVKFKYKGEEKEVDISKIKKVWRVGKMISFTYDDNGKTGRGAVSEKDAPKELLQMLEKSGKK

SEQ ID NO: 12 - protein, 64 aa, Sulfolobus shibatae
MVTVKFKYKGEEKEVDTSKIKKVWRVGKMISFTYDEGGGKTGRGAVSEKDAPKELLQMLEKQKK

SEQ ID NO: 13 - protein, 64 aa, Sulfolobus sp.
MTTVKFKYKGEEKQVDISKIKKVWRVGKMISFTYDEGGGKTGRGAVSEKDAPKELLQMLEKQKK

SEQ ID NO: 14 - protein, 64 aa, Sulfolobus sp.
MTTVKFKYKGEEKQVDTSKIKKVWRVGKMISFTYDEGGGKTGRGAVSEKDAPKELLQMLEKQKK

SEQ ID NO: 15 - protein, 64 aa, Sulfolobus shibatae
MATVKFKYKGEEKQVDISKIKKVWRVGKMISFTYDEGGGKTGRGAVSEKDAPKELLQMLEKQKK

SEQ ID NO: 16 - protein, 298 aa, Paramecium bursaria Chlorella virus 1
MAITKPLLAATLENIEDVQFPCLATPKIDGIRSVKQTQMLSRTFKPIRNSVMNRLLTELLPEGSDGEISIEGATFQDTTSAVMTGHKMYNAKFSYYWFDYVTDDPLKKYIDRVEDMKNYITVHPHILEHAQVKIIPLIPVEINNITELLQYERDVLSKGFEGVMIRKPDGKYKFGRSTLKEGILLKMKQFKDAEATIISMTALFKNTNTKTKDNFGYSKRSTHKSGKVEEDVMGSIEVDYDGVVFSIGTGFDADQRRDFWQNKESYIGKMVKFKYFEMGSKDCPRFPVFIGIRHEEDR

SEQ ID NO: 17 - protein, 305 aa, Escherichia coli
MPIIKKPLLATNVVEAASIRFPCFVTPKIDGIRAIRVDESLVSRQFKPIPNRKIRETLQNLLPDGADGEIMIPGSFRDVTSAVMTSKGTESYSKPFTFYWFDFVQTEKDAEKPYLHRMDDMKHYISKHPHIMEQSQATIIPLYPKKIESLEELVTFEQYVLDKGFEGVMIRKGDGFYKMGRSTLNEGILLKLKRFSDSEALLIRVNELFNNNNEKKATETGGWMRPTKKAGLSSSATMGSFTVRTPQNVEFNIGSGFTNEMRIKFYQEKDKLIGKIVKYKYFEIGVKNLPRFPTFLGFRDPEDIS

SEQ ID NO: 18 - DNA, 930 bp, synthetic construct
  source 1..930: mol_type = other DNA
atgcatcatcatcatcatcacggcggttcaggcaccattgcgaaacctttgttagcggcgaccttggaaaatctggatgatgtcaaattcccgtgtctggtgaccccgaaaatcgacggtatccgcagcctgaaacaacagcacatgctgagccgcaccttcaagccgatccgtaattctgtcatgaacaagctgctgagcgagttgctgccagagggcgcggacggcgagatttgcattgaagattctacgtttcaggcaaccacgagcgcagtcatgacgggtcacaaagtttacgatgaaaagttcagctattactggttcgattatgttgttgacgacccgctgaaatcgtacacggatcgtgtgaatgacatgaagaaatacgttgacgatcacccgcatatcctggagcacgaacaagtgaagatcattccgctgatcccggttgagatcaataacattgatgaactgagccagtatgagcgtgacgtcctggcgaagggtttcgagggcgtgatgatccgccgcccggacggtaagtacaagtttggtcgtagcacgctgaaggaaggcattctgctgaagatgaaacaatttaaagacgccgaggccaccattatcagcatgagcccgcgtctgaaaaacaccaatgcaaagtccaaagacaacctgggttatagcaagcgctccacccacaaatccggtaaagtcgaagaagagactatgggcagcatcgaagtggactacgacggcgtagtgtttagcattggtaccggttttgacgatgagcagcgtaagcacttctgggagaacaaagatagctacatcggtaagctcctgaagtttaagtatttcgagatgggtagcaaagatgctccgcgtttcccggtttttattggtattcgtcacgaagaggattgctaataa

SEQ ID NO: 19 - protein, 308 aa, synthetic construct
MHHHHHHGGSGTIAKPLLAATLENLDDVKFPCLVTPKIDGIRSLKQQHMLSRTFKPIRNSVMNKLLSELLPEGADGEICIEDSTFQATTSAVMTGHKVYDEKFSYYWFDYVVDDPLKSYTDRVNDMKKYVDDHPHILEHEQVKIIPLIPVEINNIDELSQYERDVLAKGFEGVMIRRPDGKYKFGRSTLKEGILLKMKQFKDAEATIISMSPRLKNTNAKSKDNLGYSKRSTHKSGKVEEETMGSIEVDYDGVVFSIGTGFDDEQRKHFWENKDSYIGKLLKFKYFEMGSKDAPRFPVFIGIRHEEDC

SEQ ID NO: 20 - protein, 307 aa, synthetic construct
MHHHHHHGGSGTIAKPLLAATLENLDDVKFPCLVTPKIDGIRSLKQQHMLSRTFKPIRNSVMNKLLSELLPEGADGEICIEDSTFQATTSAVMTGHKVYDEKFSYYWFDYVVDDPLKSYTDRVNDMKKYVDDHPHILEHEQVKIIPLIPVEINNIDELSQYERDVLAKGFEGVMIRRPDGKYKFGRSTLKEGILLKMKQFKDAEATIISMSPRLKNTNAKSKDNLGYSKRSTHKSGKVEEETMGSIEVDYDGVVFSIGTGFDDEQRKHFWENKDSYIGKLLKFKYFEMGSKDAPRFPVFIGIRHEED

SEQ ID NO: 21 - protein, 6 aa, synthetic construct
GTGGGG

SEQ ID NO: 22 - protein, 377 aa, synthetic construct
MHHHHHHGGSGATVKFKYKGEEKEVDISKIKKVWRVGKMISFTYDEGGGKTGRGAVSEKDAPKELLQMLEKQKKGTGGGGTIAKPLLAATLENLDDVKFPCLVTPKIDGIRSLKQQHMLSRTFKPIRNSVMNKLLSELLPEGADGEICIEDSTFQATTSAVMTGHKVYDEKFSYYWFDYVVDDPLKSYTDRVNDMKKYVDDHPHILEHEQVKIIPLIPVEINNIDELSQYERDVLAKGFEGVMIRRPDGKYKFGRSTLKEGILLKMKQFKDAEATIISMSPRLKNTNAKSKDNLGYSKRSTHKSGKVEEETMGSIEVDYDGVVFSIGTGFDDEQRKHFWENKDSYIGKLLKFKYFEMGSKDAPRFPVFIGIRHEEDC

SEQ ID NO: 23 - protein, 402 aa, synthetic construct
MHHHHHHGGSGNKTQLIDVIAEKADLSKSQAKAALESTLAAITESLKKGDAVQLVGFGTFKVNHRAERTGRNPQTGKEIKIAAANVPAFVSGKVLKDSVKGGSGGSTIAKPLLAATLENLDDVKFPCLVTPKIDGIRSLKQQHMLSRTFKPIRNSVMNKLLSELLPEGADGEICIEDSTFQATTSAVMTGHKVYDEKFSYYWFDYVVDDPLKSYTDRVNDMKKYVDDHPHILEHEQVKIIPLIPVEINNIDELSQYERDVLAKGFEGVMIRRPDGKYKFGRSTLKEGILLKMKQFKDAEATIISMSPRLKNTNAKSKDNLGYSKRSTHKSGKVEEETMGSIEVDYDGVVFSIGTGFDDEQRKHFWENKDSYIGKLLKFKYFEMGSKDAPRFPVFIGIRHEED

SEQ ID NO: 24 - protein, 405 aa, synthetic construct
MHHHHHHGGSGNKTQLIDVIAEKADLSKSQAKAALESTLAAITESLKKGDAVQLVGFGTFKVNHRAERTGRNPQTGKEIKIAAANVPAFVSGKVLKDSVKGGSGGSAIQKPLLAASLKKLSVDDLTFPVYATPKLDGIRALKIDGTIVSRTFKPIRNTTISNVLMSLLPDGSDGEILSGKTFQDSTSTVMSADAGIGSGTTFFWFDYVKDDPDKGYLDRIADMKKFVDSRPEILKDSRVTIVPLIPKKIDTAEELNVFEQWCLDQGFEGVMVRNAGGKYKFGRSTEKEQILVKIKQFEDDEAVVIGVSALQTNVNDKKMNELGDMRRTSHKDGKIDLEMLGALDVEWNGIRFGIGTGFDKDTREDLWKRRDSIIGKIVKFKYFSQGVKTAPRFPVFLGFRDKNDM

SEQ ID NO: 25 - protein, 90 aa, Escherichia coli
MNKTQLIDVIAEKADLSKSQAKAALESTLAAITESLKKGDAVQLVGFGTFKVNHRAERTGRNPQTGKEIKIAAANVPAFVSGKVLKDSVK

SEQ ID NO: 26 - protein, 300 aa, synthetic construct
MAIQKPLLAASLKKLSVDDLTFPVYATPKLDGIRALKIDGTIVSRTFKPIRNTTISNVLMSLLPDGSDGEILSGKTFQDSTSTVMSADAGIGSGTTFFWFDYVKDDPDKGYLDRIADMKKFVDSRPEILKDSRVTIVPLIPKKIDTAEELNVFEQWCLDQGFEGVMVRNAGGKYKFGRSTEKEQILVKIKQFEDDEAVVIGVSALQTNVNDKKMNELGDMRRTSHKDGKIDLEMLGALDVEWNGIRFGIGTGFDKDTREDLWKRRDSIIGKIVKFKYFSQGVKTAPRFPVFLGFRDKNDM